(19) United States
(12) Patent Application Publication
(10) Pub. No.: US 2003/0108891 A1
(43) Pub. Date: Jun. 12, 2003

(54) Transacylases of the paclitaxel biosynthetic pathway

(75) Inventors: Rodney B. Croteau, Pullman, WA (US); Kevin D. Walker, Pullman, WA (US); Anne Schoendorf, Collonges Sous Saleve (FR); Mark R. Wildung, Colfax, WA (US)

Correspondence Address: KLARQUIST SPARKMAN, LLP, 121 SW SALMON STREET, SUITE 1600, PORTLAND, OR, 97204, US

(73) Assignee: Washington State University Research Foundation

(21) Appl. No.: 10/166,984

(22) Filed: Sep. 18, 2002

Related U.S. Application Data
(63) Continuation-in-part of application No. 09/866,570, filed on May 25, 2001.
Application No. 09/866,570 is a division of application No. 09/457,046, filed on Dec. 7, 1999, now Pat. No. 6,287,835.
Application No. 09/457,046 is a continuation-in-part of application No. 09/411,145, filed on Sep. 30, 1999, now abandoned.

(51) Int. Cl. C12Q 1/68; C07H 21/04; C12N 9/10; C12P 21/02; C12N 5/06

(52) U.S. Cl. 435/6; 435/69.1; 435/193; 435/320.1; 435/325; 536/23.2

(57) ABSTRACT

Transacylase enzymes and the use of such enzymes to produce paclitaxel and related taxoids, as well as intermediates in the paclitaxel biosynthetic pathway, are disclosed. Also disclosed are nucleic acid sequences encoding such transacylase enzymes such as (but without limitation) C-13 phenylpropanoid side chain-CoA acyltransferase and benzoyl-CoA:3′-N-debenzoyl-2′-deoxytaxol N-benzoyltransferase.

### CROSS REFERENCE TO RELATED APPLICATIONS

This application is a continuation-in-part and claims the benefit of co-pending U.S application Ser. No. 09/866,570, filed May 25, 2001, herein incorporated by reference, which is a divisional of U.S. application Ser. No. 09/457,046, filed Dec. 7, 1999, issued as U.S. Pat. No. 6,287,835 on Sep. 11, 2001, herein incorporated by reference, which is a continuation-in-part of U.S. application Ser. No. 09/411,145, filed Sep. 30, 1999, herein incorporated by reference.

### ACKNOWLEDGMENT OF GOVERNMENT SUPPORT

This invention was made with government support under National Cancer Institute Grant No. CA-55254. The government has certain rights in this invention.

### FIELD

The invention relates to transacylase polypeptides and methods of using such polypeptides to produce paclitaxel and related taxoids.

### BACKGROUND

The complex diterpenoid Taxol® (paclitaxel) (Wani et al., J. Am. Chem. Soc. 93:2325-2327, 1971) is a potent antimitotic agent with excellent activity against a wide range of cancers, including ovarian and breast cancer (Arbuck and Blaylock, Taxol™: Science and Applications, CRC Press, Boca Raton, 397-415, 1995; Holmes et al., ACS Symposium Series 583:31-57, 1995). Taxol™ was originally isolated from the bark of the Pacific yew (Taxus brevifolia). For a number of years, Taxol® was obtained exclusively from yew bark, but low yields of this compound from the natural source coupled to the destructive nature of the harvest, prompted new methods of Taxol® production to be developed. Taxol® is currently produced primarily by semisynthesis from advanced taxane metabolites (Holton et al., Taxol™: Science and Applications, CRC Press, Boca Raton, 97-121, 1995) that are present in the needles (a renewable resource) of various Taxus species. However, because of the increasing demand for this drug (both for use earlier in the course of cancer intervention and for new therapeutic applications) (Goldspiel, Pharmacotherapy 17:110S-125S, 1997), availability and cost remain important issues. Total chemical synthesis of Taxol® is not economically feasible. Hence, biological production of the drug and its immediate precursors will remain the method of choice for the foreseeable future. Such biological production may rely upon either intact Taxus plants, Taxus cell cultures (Ketchum et al., Biotechnol. Bioeng. 62:97-105, 1999), or, potentially, microbial systems (Stierle et al., J. Nat. Prod. 58:1315-1324, 1995). In all cases, improving the biological production yields of Taxol® depends upon a detailed understanding of the biosynthetic pathway, the enzymes catalyzing the sequence of reactions, especially the rate-limiting steps, and the genes encoding these proteins. Isolation of nucleic acids encoding enzymes involved in the pathway is a particularly important goal, since overexpression of these genes in a producing organism can be expected to markedly improve yields of the drug.

The Taxol® biosynthetic pathway is considered to involve more than 12 distinct steps (Floss and Mocek, Taxol: Science and Applications, CRC Press, Boca Raton, 191-208, 1995; and Croteau et al., Curr. Top. Plant Physiol. 15:94-104, 1996), however, very few of the enzymatic reactions and intermediates of this complex pathway have been defined. The first committed enzyme of the Taxol® pathway is taxadiene synthase (Koepp et al., J. Biol. Chem. 270:8686-8690, 1995) that cyclizes the common precursor geranylgeranyl diphosphate (Hefner et al., Arch. Biochem. Biophys. 360:62-74, 1998) to taxadiene (FIG. 1). The cyclized intermediate subsequently undergoes modification involving at least eight oxygenation steps, a dehydrogenation, an epoxide rearrangement to an oxetane, and several acylations (Floss and Mocek, Taxol™: Science and Applications, CRC Press, Boca Raton, 191-208, 1995; Croteau et al., Curr. Top. Plant Physiol. 15:94-104, 1996). Taxadiene synthase has been isolated from T. brevifolia and characterized (Hezari et al., Arch. Biochem. Biophys. 322:437-444, 1995), the mechanism of action defined (Lin et al., Biochemistry 35:2968-2977, 1996), and the corresponding cDNA clone isolated and expressed (Wildung and Croteau, J. Biol. Chem. 271:9201-9204, 1996).

The second specific step of Taxol® biosynthesis is an oxygenation reaction catalyzed by taxadiene-5α-hydroxylase (FIG. 1). The enzyme, characterized as a cytochrome P450, has been demonstrated in Taxus microsome preparations to catalyze the stereospecific hydroxylation of taxa-4(5),11(12)-diene, with double bond rearrangement, to taxa-4(20),11(12)-dien-5α-ol (Hefner et al., Chem. Biol. 3:479-489, 1996).

The third specific step of Taxol® biosynthesis appears to be the acetylation of taxa-4(20),11(12)-dien-5α-ol to taxa-4(20),11(12)-dien-5α-yl acetate by an acetyl CoA-dependent transacetylase (Walker et al., Arch. Biochem. Biophys. 364:273-279, 1999), since the resulting acetate ester is then further efficiently oxygenated to a series of advanced polyhydroxylated Taxol® metabolites in microsomal preparations that have been optimized for cytochrome P450 reactions (FIG. 1). The enzyme has been isolated from induced yew cell cultures (Taxus canadensis and Taxus cuspidata), and the operationally soluble enzyme lo was partially purified by a combination of anion exchange, hydrophobic interaction, and affinity chromatography on immobilized coenzyme A resin. This acetyl transacylase has a pI and pH optimum of 4.7 and 9.0, respectively, and a molecular weight of about 50,000 as determined by gel-permeation chromatography. The enzyme shows high selectivity and high affinity for both cosubstrates with Kₘ values of 4.2 μM and 5.5 μM for taxadienol and acetyl CoA, respectively. The enzyme does not acetylate the more advanced Taxol® precursors, 10-deacetylbaccatin III or baccatin III. This acetyl transacylase is insensitive to monovalent and divalent metal ions, is only weakly inhibited by thiol-directed reagents and Co-enzyme A, and in general displays properties similar to those of other O-acetyl transacylases. This acetyl CoA:taxadien-5α-ol O-acetyl transacylase from Taxus (Walker et al., Arch. Biochem. 20 Biophys. 364:273-279, 1999) appears to be substantially different in size, substrate selectivity, and kinetics from an acetyl CoA: 10-hydroxytaxane O-acetyl transacylase recently isolated and described from Taxus chinensis (Menhard and Zenk, Phytochemistry 50:763-774, 1999).

Acquisition of the nucleic acid encoding the acetyl CoA:taxa-4(20),11(12)-dien-5α-ol O-acetyl transacylase that catalyzes the first acylation step of Taxol® biosynthesis and genes encoding other acyl transfer steps directed to the taxane core and to side chain assembly would represent important advances in efforts to increase Taxol® yields by genetic engineering and in vitro synthesis.

### SUMMARY

Disclosed are twelve amplicons (regions of DNA amplified by a pair of primers using the polymerase chain reaction (PCR)). These amplicons can be used to identify transacylases, for example, the transacylases shown in SEQ ID NOs: 26, 28, 45, 50, 52, 54, 56, and 58 that are encoded by the nucleic acid sequences shown in SEQ ID NOs: 25, 27, 44, 49, 51, 53, 55, and 57. These sequences are isolated from the Taxus genus, and the respective transacylases are useful for the synthetic production of Taxol® and related taxoids, as well as intermediates within the Taxol® biosynthetic pathway. The sequences also can be used for the creation of transgenic organisms that either produce the transacylases for subsequent in vitro use, or produce the transacylases in vivo so as to alter the level of Taxol® and taxoid production within the transgenic organism.

The nucleic acid sequences shown in SEQ ID NOs: 1, 3, 5, 7, 9, 11, 13, 15, 17, 19, 21, and 23 and the corresponding amino acid sequences shown in SEQ ID NOs: 2, 4, 6, 8, 10, 12, 14, 16, 18, 20, 22, and 24, as well as fragments of the nucleic acid and the amino acid sequences are also provided. These sequences are useful for isolating the nucleic acid and amino acid sequences corresponding to full-length transacylases. These amino acid sequences and nucleic acid sequences also are useful for creating specific binding agents that recognize the corresponding transacylases.

In some embodiments, transacylases and fragments of transacylases that have amino acid and nucleic acid sequences that vary from the disclosed sequences are identified. As one non-limiting example, the disclosed amino acid sequences can be used to identify other transacylase polypeptides that vary by one or more conservative amino acid substitutions from, or that share at least 50% sequence identity with, the disclosed amino acid sequences. In specific embodiments, the other transacylase polypeptides that are identified maintain transacylase activity.

The nucleic acid sequences encoding the transacylases and fragments of the transacylases can be cloned into vectors, using standard molecular biology techniques. These vectors can then be used to transform host cells. Thus, a host cell can be modified to express either increased levels of transacylase or decreased levels of transacylase.

Also disclosed are methods for isolating nucleic acid sequences encoding full-length transacylases. The methods involve hybridizing at least ten contiguous nucleotides of any of the nucleic acid sequences shown in SEQ ID NOs: 1, 3, 5, 7, 9, 11, 13, 15, 17, 19, 21, 23, 25, 27, 44, 49, 51, 53, 55, and 57 to a second nucleic acid sequence, wherein the second nucleic acid sequence encodes a transacylase. This method can be practiced in the context of, for example, Northern blots, Southern blots, and the polymerase chain reaction (PCR). Therefore, some embodiments include transacylase nucleic acids and polypeptides identified by such a method.

Furthermore, methods of adding at least one acyl group to at least one taxoid or taxoid side chain are provided. These methods can be practiced in vivo or in vitro, and can be used to add acyl groups to various intermediates in the Taxol® biosynthetic pathway, and to add acyl groups to related taxoids or taxoid side chains that are not necessarily in a Taxol® biosynthetic pathway.

### BRIEF DESCRIPTION OF THE SEQUENCE LISTING

The nucleic acid and amino acid sequences listed herein are shown using standard letter abbreviations for nucleotide bases and the standard one- or three-letter abbreviations for amino acid residues. Only one strand of each nucleic acid sequence is shown, but the complementary strand is understood as included by any reference to the displayed strand.

SEQ ID NO: 1 is the nucleotide sequence of Probe 1.

SEQ ID NO: 2 is the deduced amino acid sequence of Probe 1.

SEQ ID NO: 3 is the nucleotide sequence of Probe 2.

SEQ ID NO: 4 is the deduced amino acid sequence of Probe 2.

SEQ ID NO: 5 is the nucleotide sequence of Probe 3.

SEQ ID NO: 6 is the deduced amino acid sequence of Probe 3.

SEQ ID NO: 7 is the nucleotide sequence of Probe 4.

SEQ ID NO: 8 is the deduced amino acid sequence of Probe 4.

SEQ ID NO: 9 is the nucleotide sequence of Probe 5.

SEQ ID NO: 10 is the deduced amino acid sequence of Probe 5.

SEQ ID NO: 11 is the nucleotide sequence of Probe 6.

SEQ ID NO: 12 is the deduced amino acid sequence of Probe 6.

SEQ ID NO: 13 is the nucleotide sequence of Probe 7.

SEQ ID NO: 14 is the deduced amino acid sequence of Probe 7.

SEQ ID NO: 15 is the nucleotide sequence of Probe 8.

SEQ ID NO: 16 is the deduced amino acid sequence of Probe 8.

SEQ ID NO: 17 is the nucleotide sequence of Probe 9.

SEQ ID NO: 18 is the deduced amino acid sequence of Probe 9.

SEQ ID NO: 19 is the nucleotide sequence of Probe 10.

SEQ ID NO: 20 is the deduced amino acid sequence of Probe 10.

SEQ ID NO: 21 is the nucleotide sequence of Probe 11.

SEQ ID NO: 22 is the deduced amino acid sequence of Probe 11.

SEQ ID NO: 23 is the nucleotide sequence of Probe 12.

SEQ ID NO: 24 is the deduced amino acid sequence of Probe 12.

SEQ ID NO: 25 is the nucleotide sequence of the full-length acyltransacylase clone TAX2.

SEQ ID NO: 26 is the deduced amino acid sequence of the full-length acyltransacylase clone TAX2.

SEQ ID NO: 27 is the nucleotide sequence of the full-length acyltransacylase clone TAX1.

SEQ ID NO: 28 is the deduced amino acid sequence of the full-length acyltransacylase clone TAX1.

SEQ ID NO: 29 is the amino acid sequence of a transacylase peptide fragment.

SEQ ID NO: 30 is the amino acid sequence of a transacylase peptide fragment.

SEQ ID NO: 31 is the amino acid sequence of a transacylase peptide fragment.

SEQ ID NO: 32 is the amino acid sequence of a transacylase peptide fragment.

SEQ ID NO: 33 is the amino acid sequence of a transacylase peptide fragment.

SEQ ID NO: 34 is the AT-FOR1 PCR primer.

SEQ ID NO: 35 is the AT-FOR2 PCR primer.

SEQ ID NO: 36 is the AT-FOR3 PCR primer.

SEQ ID NO: 37 is the AT-FOR4 PCR primer.

SEQ ID NO: 38 is the AT-REV1 PCR primer.

SEQ ID NO: 39 is an amino acid sequence variant that allowed for the design of the AT-FOR3 PCR primer.

SEQ ID NO: 40 is an amino acid sequence variant that allowed for the design of the AT-FOR4 PCR primer.

SEQ ID NO: 41 is a consensus amino acid sequence that allowed for the design of the AT-REV1 PCR primer.

SEQ ID NO: 42 is a PCR primer, useful for identifying transacylases.

SEQ ID NO: 43 is a PCR primer, useful for identifying transacylases.

SEQ ID NO: 44 is the nucleotide sequence of the full-length acyltransacylase clone TAX6.

SEQ ID NO: 45 is the deduced amino acid sequence of the full-length acyltransacylase clone TAX6.

SEQ ID NO: 46 is a PCR primer, useful for identifying TAX6.

SEQ ID NO: 47 is a PCR primer, useful for identifying TAX6.

SEQ ID NO: 48 is a 6-amino acid motif commonly found in transacylases.

SEQ ID NO: 49 is the nucleotide sequence of the full-length acyltransacylase clone TAX5.

SEQ ID NO: 50 is the deduced amino acid sequence of the full-length acyltransacylase clone TAX5.

SEQ ID NO: 51 is the nucleotide sequence of the full-length acyltransacylase clone TAX7.

SEQ ID NO: 52 is the deduced amino acid sequence of the full-length acyltransacylase clone TAX7.

SEQ ID NO: 53 is the nucleotide sequence of the full-length acyltransacylase clone TAX10.

SEQ ID NO: 54 is the deduced amino acid sequence of the full-length acyltransacylase clone TAX10.

SEQ ID NO: 55 is the nucleotide sequence of the full-length acyltransacylase clone TAX12.

SEQ ID NO: 56 is the deduced amino acid sequence of the full-length acyltransacylase clone TAX12.

SEQ ID NO: 57 is the nucleotide sequence of the full-length acyltransacylase clone TAX13.

SEQ ID NO: 58 is the deduced amino acid sequence of the full-length acyltransacylase clone TAX13.

SEQ ID NO: 59 is the nucleotide sequence of the full-length acyltransacylase clone TAX9.

SEQ ID NO: 60 is the deduced amino acid sequence of the full-length acyltransacylase clone TAX9.

SEQ ID NO: 61-76 are the amino acid sequences shown in FIG. 6.

### BRIEF DESCRIPTION OF THE DRAWINGS

FIG. 1 is an illustration of the enzymatic reactions of the Taxol® pathway indicating cyclization of geranylgeranyl diphosphate to taxa-4(5),11(12)-diene, followed by hydroxylation/rearrangement and acetylation to taxa-4(20),11(12)-dien-5α-yl acetate. The acetate is further converted to 10-deacetylbaccatin III, baccatin III, and Taxol®. In the figure, the “a” reaction is catalyzed by taxadiene synthase; the “b” reaction is catalyzed by taxadiene-5α-hydroxylase; the “c” reaction is catalyzed by taxadien-5α-ol acetyl transacylase; the “d” reaction is catalyzed by a 10-deacetylbaccatin III acetyl transacylase and “e” reactions are catalyzed by several side chain assembly enzymes. The latter two steps are further described below.

FIG. 2 is a table of peptide sequences generated by endolysC and trypsin proteolysis of purified taxadienol acetyl transacylase.

FIGS. 3A-3C are graphs showing FPLC elution profiles, while FIG. 3D is a digitized image showing purity of taxadien-5α-ol acetyl transacylase after hydroxyapatite chromatography. FIG. 3A is an elution profile of the acetyl transacylase on Source HR 15Q (10×100 mm) preparative scale anion-exchange chromatography; FIG. 3B is an elution profile on analytical scale Source HR 15Q (5×50 mm) column chromatography; and FIG. 3C is an elution profile on the ceramic hydroxyapatite column. In FIGS. 3A-3C, the solid line is the UV absorbance at 280 nm; the dotted line is the relative transacetylase activity (dpm); and the hatched line is the elution gradient (sodium chloride or sodium phosphate). FIG. 3D is a digitized image of a photograph of a silver-stained 12% SDS-PAGE showing the purity of taxadien-5α-ol acetyl transacylase (˜50 kDa) after hydroxyapatite chromatography. A minor contaminant is present at ˜35 kDa.

FIG. 4 is an illustration of the four forward (AT-FOR1, AT-FOR2, AT-FOR3, AT-FOR4; SEQ ID NOS: 34-37) and one reverse (AT-REV1; SEQ ID NO: 38) degenerate primers that were used to amplify an induced Taxus cell library cDNA from which twelve hybridization probes were obtained. Inosine positions are indicated by “I”. Each of the forward primers was paired with the reverse primer in separate PCR reactions. Primers AT-FOR1 (SEQ ID NO: 34) and AT-FOR2 (SEQ ID NO: 35) were designed from the tryptic fragment SEQ ID NO: 30; the remaining primers were derived by database searching based on SEQ ID NO: 30.

FIGS. 5A-5G are graphs illustrating data obtained from a coupled gas chromatographic-mass spectrometric (GC-MS) analysis of the biosynthetic taxadien-5α-yl acetate formed during the incubation of taxadien-5α-ol with soluble enzyme extracts from isopropyl β-D-thiogalactoside (IPTG)-induced E. coli JM109 cells transformed with full-length acyltransacylase clones TAX1 (SEQ ID NO: 27) and TAX2 (SEQ ID NO: 25). FIGS. 5A and 5B show the respective GC and MS profiles of authentic taxadien-5α-ol; FIGS. 5C and 5D show the respective GC and MS profiles of authentic taxadien-5α-yl acetate; FIG. 5E shows the GC profile of taxadien-5α-ol (11.16 minutes), taxadien-5α-yl acetate (11.82 minutes), dehydrated taxadien-5α-ol (“TOH-H₂O” peak), and a contaminant, bis-(2-ethylhexyl)phthlate (“BEHP” peak, a plasticizer, CAS 117-81-7, extracted from buffer) after incubation of taxadien-5α-ol and acetyl coenzyme A with the soluble polypeptide fraction derived from E. coli JM109 transformed with the full-length clone TAX1. FIG. 5F shows the mass spectrum of biosynthetically formed taxadien-5α-yl acetate by the recombinant enzyme (11.82 minute peak in GC profile FIG. 5E); FIG. 5G shows the GC profile of the products generated from taxadien-5α-ol and acetyl coenzyme A by incubation with the soluble enzyme fraction derived from E. Coli JM109 cells transformed with the full-length clone TAX2 (SEQ ID NO: 25) (the absence of taxadien-5α-yl acetate indicates that this clone is inactive in the transacylase reaction).

FIGS. 6A-6N illustrate a sequence alignment of the deduced amino acid sequences listed in Table 2, and of TAX1 (SEQ. ID NO: 28) and TAX2 (SEQ ID NO: 26). Residues underlined in bold italics indicate the few regions of conservation. Forward arrow (left to right) shows conserved region from which degenerate forward PCR primers were designed. Reverse arrow (right to left) shows region from which the reverse PCR primer was designed (cf, FIG. 4).

FIG. 7 is a dendrogram showing deduced peptide sequence relationships between Taxus transacylase sequences (Probes 1-12, TAX1 (SEQ. ID NO: 28), and TAX2 (SEQ ID NO: 26)) and closest relative sequences of defined and unknown function obtained from the GenBank database described in Table 2.

FIGS. 8A-8B illustrate a biosynthetic scheme for the formation of the oxetane, present in Taxol® and related late-stage taxoids. FIG. 8A shows the outline of the Taxol® biosynthetic pathway. The cyclization of geranylgeranyl diphosphate to taxadiene by taxadiene synthase, and the hydroxylation to taxadien-5α-ol by taxadiene 5-α-hydroxylase (a), the acetylation of taxadien-5α-ol by taxa-4(20),11(12)-dien-5α-ol-O-acetyl transferase (b), the conversion of 10-deacetylbaccatin III to baccatin III by 10-deacetylbaccatin III-10-O-acetyl transferase (c), and the side chain attachment to baccatin III to form Taxol® (d) are highlighted. The broken arrow indicates several as yet undefined steps. FIG. 8B shows a postulate biosynthetic scheme for the formation of the oxetane, present in Taxol® and related late-stage taxoids, in which the 4(20)-ene-5α-ol is converted to the 4(20)-ene-5α-yl acetate followed by epoxidation to the 4(20)-epoxy-5α-acetoxy group and then intramolecular rearrangement to the 4-acetoxy oxetane moiety.

FIGS. 9A-9B are graphs illustrating radio-HPLC (high-performance liquid chromatography) analysis of the biosynthetic product (Rₜ=7.0±0.1 minutes) generated from 10-deacetylbaccatin III and [2-³H]acetyl CoA by the recombinant acetyl transferase (polypeptide expressed from TAX6 (SEQ ID NO: 45)). The top trace (FIG. 9A) shows the UV profile and the bottom trace (FIG. 9B) shows the coincident radioactivity profile, both of which coincide with the retention time of authentic baccatin III. For the enzyme preparation, E. coli cells transformed with the pCWori+ vector harboring the putative DBAT nucleic acid (TAX6) were grown overnight at 37° C. in 5 mL Luria-Bertani medium supplemented with ampicillin, and 1 mL of this inoculum was added to and grown in 100 mL Terrific Broth culture medium (6 g bacto-tryptone, Difco Laboratories, Spark, Md., 12 g yeast extract, EM Science, Cherryhill, N.J., and 2 mL gycerol in 500 mL water) supplemented with 1 mM IPTG, 1 mM thiamine HCl and 50 μg ampicillin/mL. After 24 hours, the bacteria were harvested by centrifugation, resuspended in 20 mL of assay buffer (25 mM Mopso, pH 7.4) and then disrupted by sonication at 0-4° C. The resulting homogenate was centrifuged at 15,000 g to remove debris, and a 1 mL aliquot of the supernatant was incubated with 10-deacetylbaccatin III (400 μM) and [2-³H]acetyl coenzyme A (0.45 μCi, 400 μM) for 1 hour at 31°0 C. The reaction mixture was then extracted with ether and the solvent concentrated in vacuo. The crude product (pooled from five such assays) was purified by silica gel thin-layer chromatography (TLC; 70:30 ethyl acetate: hexane). The band co-migrating with authentic baccatin III (Rf=0.45 for the standard) was isolated and analyzed by radio-HPLC to reveal the new radioactive product described herein. Extracts of E. coli transformed with empty vector controls did not yield detectable product when assayed by identical methods.

FIGS. 10A-10B are graphs illustrating the combined reverse-phase HPLC-chemical ionization MS (mass spectrometry) analysis of the biosynthetic product (Rₜ=8.6±0.1 minutes) generated by recombinant acetyl transferase with 10-deaceylbaccatin III and acetyl CoA as co-substrates (FIG. 10A), and of authentic baccatin III (Rₜ=8.6±0.1 minutes) (FIG. 10B). The diagnostic mass spectral fragments are at m/z 605 (M+NH₄⁺), 587 (MH⁺), 572 (MH⁺—CH₃), 527 (MH⁺—CH₃COOH), and 509 (MH⁺—CH₃COOH—H₂O). For preparation of recombinant enzyme and product isolation, see FIG. 9 description.

FIGS. 11A-11B illustrate the progression of advanced metabolites in the Taxol® biosynthetic pathway (cf., FIG. 1). Shown in FIG. 11A are the acetylation of 10-deacetylbaccatin III to baccatin III by 10-deacetylbaccatin III O-acetyltransferase (a), the transfer of an aminophenylpropanoyl group to C-13 of baccatin III by an O-(3-amino-3-phenylpropanoyl)transferase (b), and the benzamidation and C-2′ hydroxylation of the side chain by N-debenzoyltaxol N-benzoyltransferase and a taxoid P450 hydroxylase (c). Shown in FIG. 11B are the synthesized phenylpropanoyl coenzyme A esters used for specificity analysis of the O-(3-amino-3-phenylpropanoyl)transferase.

FIG. 12 is an illustration of the semisynthesis of Taxol® from the natural product 10-deacetylbaccatin III and a synthetic β-lactam precursor of the N-benzoyl phenylisoserine side chain.

FIGS. 13A-13B illustrate radio-HPLC analysis after chemical N-benzoylation of the biosynthetic product (Rₜ=39.6±0.1 min) generated by the recombinant O-phenylpropanoyltransferase using [13-³H]baccatin III and β-phenylalanoyl-CoA as cosubstrates. The Upper trace (FIG. 13A) shows the radioactivity profile (in mV) and the Lower trace (FIG. 13B) shows the absorbance profile (A₂₅₄), both of which coincide exactly with the retention time of authentic (3′RS)-2′-deoxyltaxol.

FIG. 14 is a partial ¹H-NMR spectra (recorded in deuterated chloroform) of authentic (3′RS)-2′-deoxyltaxol (Upper) and of the biosynthetic product, after chemical N-benzoylation, derived by the recombinant O-phenylpropanoyltransferase (TAX7) with baccatin III and β-phenylalaninoyl-CoA as cosubstrates (Lower). The complete spectrum of the N-benzoylated biosynthetic product was identical to that of the 2′-deoxytaxol standard.

FIGS. 15A-15B are spectra illustrating the coupled reversed-phase HPLC APCI-MS analysis of the biosynthetic product (after chemical N-benzoylation) (Rₜ=20.8±0.1) generated by the recombinant phenylpropanoyltransferase with baccatin III and β-phenylalanoyl-CoA as co-substrates. The biosynthetic product demonstrated a retention time virtually identical to that of authentic (3′RS)-2′-deoxytaxol (FIG. 15A). Diagnostic ions are m/z 838 (PH⁺), 820 (PH⁺—H₂O), 778 (PH⁺—CH₃CO₂H), 760 (m/z 778-H₂O), 569 (PH^{+—PhCH(BzNH)CH}₂CO₂H), 509 (m/z 569-CH₃CO₂H), and 270 (PhCH(BzNH)CH₂CO₂H) (FIG. 15B).

FIG. 16 illustrates a comparison, and the deduced sequence alignment, among several amino acid sequences: 3-amino-3-phenylpropanoyltransferase (BAPT, accession no. AY082804), taxadien-5α-ol O-acetyltransferase (TAT, accession no. AF190130), taxane 2α-O-benzoyltransferase (TBT, accession no. AF297618), 10-deacetylbaccatin III 10-O-acetyltransferase (DBAT, accession no. AF193765), and N-debenzoyltaxol N-benzoyltransferase (DBTNBT, accession no. AF466397). Columns with residues in black boxes indicate identical amino acids for at least three of the compared sequences, while columns with residues in gray boxes indicate similar amino acids for at least four of the compared sequences.

FIG. 17 is an illustration of part of the Taxol® biosynthetic pathway. Shown are the cyclization of geranylgeranyl diphosphate to taxadiene by taxadiene synthase and the hydroxylation to taxadien-5α-ol by taxadiene 5α-hydroxylase (a), the acetylation of taxadien-5α-ol by taxadien-5α-ol acetyltransferase (TAT) (b), the conversion of taxadien-5α-ol to 5,13-diol by a 13α-hydroxylase (c), the hydroxylation of taxadien-5α-yl acetate by a 10β-hydroxylase (d), the formation of a 2-benzoxy taxoid by a taxane 2α-O-benzoyltransferase (TBT) (e), the conversion of 10-deacetylbaccatin III to baccatin III by a 10-O-acetyltransferase (DBAT) (f), sidechain attachment by a 13-O-[3-amino-3-phenylpropanyl]transferase (g), and sidechain benzamidation by N-debenzoyltaxol N-benzoyltransferase (DBTNBT) and hydroxylation to form Taxol® (h).

FIGS. 18A-18B are spectra illustrating the radio-HPLC analysis of the biosynthetic product (retention time Rₜ=39.6±0.1 min) catalyzed by the recombinant N-benzoyltransferase using (3′RS)-N-debenzoyl-2′-deoxytaxol and [7-¹⁴C]benzoyl-CoA as cosubstrates. FIG. 1 8A shows the radioactivity profile (in mV) and FIG. 18B shows the UV profile (A₂₅₄), both of which coincide exactly with the retention time of authentic (3′RS)-2′-deoxytaxol.

FIG. 19 is a partial ¹H-NMR spectra (recorded in deuterochloroform) of authentic (3′RS)-2′-deoxyltaxol (Upper trace) and of the biosynthetic product derived by the recombinant N-benzoyltransferase (TAX10) with (3′RS)-N-debenzoyl-2′-deoxytaxol and benzoyl coenzyme-A as cosubstrates (Lower trace).

FIG. 20 is a graph illustrating the APCI-MS analysis of the biosynthetic product generated by the recombinant N-benzoyltransferase with (3′RS)-N-debenzoyl-2′-deoxytaxol and benzoyl coenzyme-A as cosubstrates. Diagnostic ions are at m/z 855 (P⁺+H₂O), 838 (PH⁺), 820 (PH⁺—H₂O), 778 (PH⁺—CH₃CO₂H), 760 (m/z 778-H₂O), 569 (PH⁺—PhCH(BzNH)CH₂CO₂H), 509 (m/z 569-CH₃CO₂H), and 270 (PhCH(BzNH)CH₂CO₂H+H⁺).

FIGS. 21A-21B illustrate a comparison, and the deduced sequence alignment, among several amino acid sequences: DBTNBT (accession no. AF466397), anthranilate N-hydroxycinnamoyl/benzoyltransferase (PCHCBT, accession no. Z84383), taxane 2α-O-benzoyltransferase (TBT, accession no. AF297618), 10-deacetylbaccatin III 10-O-acetyltransferase (DBAT, accession no. AF193765), taxadien-5α-ol O-acetyltransferase (TAT, accession no. AF190130), deacetylvindoline 4-O-acetyltransferase (DAT, accession no. AF053307), benzyl alcohol acetyltransferase (BEAT, accession no. AF043464), and salutaridinol 7-O-acetyltransferase (SALAT, accession no. AF339913). Columns with residues in black boxes indicate identical amino acids for at least four of the compared sequences while columns containing at least four similar amino acids are indicated by gray shading.

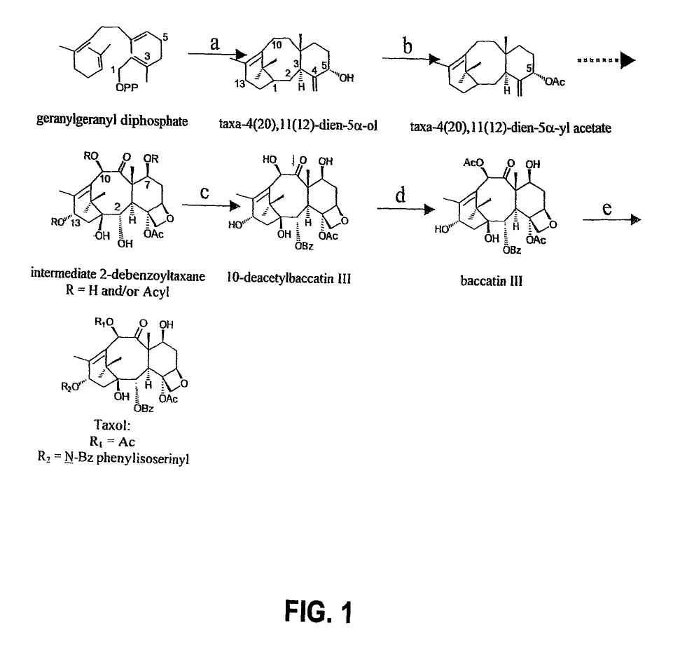

### DETAILED DESCRIPTION

Abbreviations

aa=amino acid

BEAT=benzyl alcohol acetyltransferase

bp=base pair

cDNA=copy DNA

Da=Dalton

DAT=deacetylvindoline 4-O-acetyltransferase

DBAT=10-deacetylbaccatin III 10-O-acetyltransferase

DBTNBT=and N-debenzoyltaxol N-benzoyltransferase

ELISA=enzyme-linked immunosorbent assay

HPLC high-performance liquid chromatography

kbp=kilo-base pair

kDa=kilo-Dalton

nt=nucleotide

orf=open reading frame

PAGE=polyacrylamide gel electrophoresis

PCHCBT=anthranilate N-hydroxycinnamoyl/benzoyltransferase

PCR=polymerase chain reaction

RT=reverse transcription

RT-PCR=reverse transcriptase PCR

SALAT=salutaridinol 7-O-acetyltransferase

SDS-PAGE=sodium dodecyl sulfate polyacrylamide gel electrophoresis

TAT=taxadien-5α-ol O-acetyltransferase

TBT=taxane 2α-O-benzoyltransferase

Explanations of Terms

Unless otherwise noted, technical terms are used according to conventional usage. Definitions of common terms in molecular biology and biochemistry can be found in Lewin, Genes VII (Oxford University Press, 1999); Kendrew et al. (eds.), The Encyclopedia of Molecular Biology (Blackwell Science Ltd., 1994); Robert A. Meyers (ed.), Molecular Biology and Biotechnology: a Comprehensive Desk Reference (VCH Publishers, Inc., 1995); and Nelson and Cox, Lehninger: Principles of Biochemistry, 3^{rd} Ed. (Worth Publishing, 2000).

The singular forms “a,” “an,” and “the” refer to one or more than one, unless the context clearly dictates otherwise. For example, the term “comprising a nucleic acid” includes single or plural nucleic acids and is considered equivalent to the phrase “comprising at least one nucleic acid.”

The term “or” refers to a single element of stated alternative elements or a combination of two or more elements. For example, the phrase “a first nucleic acid or a second nucleic acid” refers to the first nucleic acid, the second nucleic acid, or both the first and second nucleic acids.

As used herein, “comprises” means “includes.” Thus, “comprising A and B” means “including A and B,” without excluding additional elements.

Amplification: Regarding a nucleic acid molecule (such as a DNA or RNA molecule), amplification refers to use of a technique that increases the number of copies of a nucleic acid molecule in a specimen. An example of amplification is the polymerase chain reaction (PCR), in which a biological sample collected from a subject is contacted with a pair of oligonucleotide primers under conditions that allow for the hybridization of the primers to nucleic acid template in the sample. The primers are extended under suitable conditions, dissociated from the template, and then re-annealed, extended, and dissociated to amplify the number of copies of the nucleic acid. The product of amplification can be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

Antisense: The natural mechanism for producing proteins in living cells starts with the DNA being transcribed into RNA. The resulting RNA molecule is then translated into a protein. This chain of events (DNA→RNA→Protein) allows for the regulation of the protein at three different levels. At the first level of regulation the DNA can be targeted. This is done such that the process of making the RNA is inhibited. For example, a small circular oligonucleotide molecule can be placed in contact with the DNA thus inhibiting and/or altering transcription (Wolf, Nature Biotechnology 16:341-344, 1998). At the next level the transcription of the RNA can be inhibited. This can be done through the use of complementary polynucleotide sequences that bind to the target RNA molecule. In some instances, these polynucleotide molecules can be designed so that they are catalytic. In other words, they can be designed so that they can bind to a first target RNA, cleave it, and then move on to cleave a second RNA. Finally, at the third level, the protein itself can be regulated through the use of antibodies and other therapeutic molecules.

The use of complementary polynucleotide sequences is referred to as antisense technology. Therefore, these polynucleotide molecules are commonly called antisense molecules, and these antisense molecules can be designed and produced in many different ways using the same techniques for cloning, producing, or synthesizing a nucleic acid molecule.

cDNA (complementary DNA): A “cDNA” is a piece of DNA lacking internal, non-coding segments (introns) and regulatory sequences that determine transcription. cDNA can be synthesized in the laboratory, such as by reverse transcription from messenger RNA extracted from cells.

DNA construct: The term “DNA construct” is intended to indicate any nucleic acid molecule of cDNA, genomic DNA, synthetic DNA, or RNA origin. The term “construct” is intended to indicate a nucleic acid segment that is either single- or double-stranded, and that can be based on a complete or partial naturally occurring nucleotide sequence encoding one or more of the transacylase nucleic acids disclosed herein. It is understood that such nucleotide sequences include intentionally manipulated nucleotide sequences, e.g., subjected to site-directed mutagenesis, and sequences that are degenerate as a result of the genetic code. All degenerate nucleotide sequences are included, so long as the transacylase encoded by the nucleotide sequence maintains transacylase activity.

Expression control sequence. A nucleic acid sequence that affects, modifies, or influences expression of a second nucleic acid sequence. Promoters, operators, repressors, and enhancers are examples of expression control sequences. In some embodiments, the expression control sequence is a promoter, such as a cytomegalovirus (CMV) promoter.

Homologs: “Homologs” are two nucleotide sequences that share a common ancestral sequence and diverged when a species carrying that ancestral sequence split into two species.

Isolated: An “isolated” biological component (such as a nucleic acid, protein, metabolite, organic compound, or organelle) is a component that has been substantially separated or purified away from other biological components in the cell of the organism in which the component naturally occurs, i.e., other chromosomal and extra-chromosomal DNA, RNA, proteins, metabolite, organic compounds, and organelles. Nucleic acids and proteins that have been “isolated” include nucleic acids and proteins purified by standard purification methods. The term also embraces nucleic acids and proteins prepared by recombinant expression in a host cell, as well as chemically synthesized nucleic acids.

Mammal: This term includes both human and non-human mammals. Similarly, the term “patient” includes both humans and veterinary subjects.

Nucleic acid. A deoxyribonucleotide or ribonucleotide polymer in either single or double stranded form. Unless otherwise limited, this term encompasses known analogues of natural nucleotides that hybridize to nucleic acids in a manner similar to naturally occurring nucleotides. An “oligonucleotide” (or “oligo”) is a linear nucleic acid of up to about 250 nucleotide bases in length. For example, a nucleic acid (such as DNA or RNA) can be at least 5 nucleotides long, such as at least 15, 50, 100, 200, or even more than 500 nucleotides long, such as more than 900, 1000, or 1200 nucleotides long, or even longer. In particular embodiments, however, the nucleic acid has a length of about 1500 nucleotides or less.

ORF (open reading frame): An “ORF” is a series of nucleotide triplets (codons) coding for amino acids without any internal termination codons. These sequences are usually translatable into respective polypeptides.

Operably linked: A first nucleic acid sequence is “operably linked” with a second nucleic acid sequence whenever the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous and, where necessary to join two protein-coding regions, in the same reading frame.

Orthologs: An “ortholog” is a nucleic acid that encodes a protein that displays a function that is similar to a nucleic acid derived from a different species (i.e., are likely to be homologous).

Probes and primers: Nucleic acid probes and primers can be prepared readily based on the amino acid sequences and nucleic acid sequences disclosed herein. A “probe” comprises an isolated nucleic acid attached to a detectable label or reporter molecule. Typical labels include radioactive isotopes, ligands, chemiluminescent agents, and enzymes. Methods for labeling and guidance in the choice of labels appropriate for various purposes are discussed in, e.g., Sambrook et al., Molecular Cloning: A Laboratory Manual (Cold Spring Harbor Laboratory Press, Cold Spring Harbor, N.Y., 2001), and Ausubel et al. (eds.) Current Protocols in Molecular Biology, Greene Publishing and Wiley-Interscience, New York (with periodic updates), 1987.

“Primers” are short nucleic acids, such as DNA oligonucleotides of 10 nucleotides or more in length. A primer can be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR), or other nucleic-acid amplification methods known in the art.

Methods for preparing and using probes and primers are described, for example, in references such as Innis, et al., PCR Applications: Protocols for Functional Genomics, Academic Press, San Diego, 1999; Sambrook et al., 2001; and Ausubel et al., 1987. PCR primer pairs can be derived from a known sequence, for example, by using computer programs intended for that purpose such as Primer3 (Version 0.6, © 1998, Whitehead Institute for Biomedical Research, Cambridge, Mass.). The specificity of a particular probe or primer increases with the length of the probe or primer. Thus, for example, a primer comprising 20 consecutive nucleotides will anneal to a target with a higher specificity than a corresponding primer of only 15 nucleotides. Thus, in order to obtain greater specificity, probes and primers can be selected that comprise, for example, 10, 15, 20, 25, 30, 35, 40, 50 or more consecutive nucleotides.

Promoter. A promoter is one type of expression control sequence composed from an array of nucleic acid sequences that directs transcription of a nucleic acid. A promoter includes necessary nucleic acid sequences near the start site of transcription, such as a TATA element. A promoter also can include distal enhancer or repressor elements that can be located as much as several thousand base pairs from the start site of transcription.

An inducible promoter directs transcription of a nucleic acid operably linked to it only under certain environmental conditions, such as in the presence of metal ions or above a certain temperature. A constitutive promoter directs transcription of a nucleic acid operably linked to it in a substantially constant manner.

Purified: The term “purified” does not require absolute purity; rather, it is intended as a relative term. Thus, for example, a purified enzyme, nucleic acid, or organic compound preparation is one in which the subject protein, nucleotide, or compound is at a higher relative concentration than the protein, nucleotide or compound would be in its natural environment within an organism. For example, a preparation of an enzyme can be considered as purified if the enzyme content in the preparation represents at least 50% of the total protein content of the preparation.

Recombinant: A “recombinant” nucleic acid is one having a sequence that is not naturally occurring in the organism in which it is expressed, or has a sequence made by an artificial combination of two otherwise separated sequences. This artificial combination is often accomplished by chemical synthesis or, more commonly, by the artificial manipulation of isolated segments of nucleic acids, e.g., by genetic engineering techniques. “Recombinant” is also used to describe nucleic acid molecules that have been artificially manipulated, but contain the same control sequences and coding regions that are found in the organism from which the nucleic acid was isolated.

Specific binding agent: A “specific binding agent” is an agent that is capable of specifically binding to the transacylases disclosed herein, and can include polyclonal antibodies, monoclonal antibodies (including humanized monoclonal antibodies) and fragments of monoclonal antibodies such as Fab, F(ab′)2 and Fv fragments, as well as any other agent capable of specifically binding to the epitopes on the proteins.

Sequence identity: The similarity between two nucleic acid sequences or between two amino acid sequences can be expressed in terms of the level of sequence identity shared between the sequences. Sequence identity is typically expressed in terms of percentage identity; the higher the percentage, the more similar the two sequences.

Methods for aligning sequences, including various programs and alignment algorithms, are known. See, e.g., Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene 73:237-244, 1988; Higgins & Sharp, CABIOS 5:151-153, 1989; Corpet et al., Nucleic Acids Research 16:10881-10890, 1988; Huang, et al., CABIOS 8:155-165, 1992; and Pearson et al., Methods in Molecular Biology 24:307-331, 1994. Altschul et al., J. Mol. Biol. 215:403-410, 1990, presents a detailed consideration of sequence alignment methods and homology calculations.

The National Center for Biotechnology Information (NCBI) Basic Local Alignment Search Tool (BLAST™, Altschul et al.. J. Mol. Biol. 215:403-410, 1990) is available from several sources, including the National Center for Biotechnology Information (NCBI, Bethesda, Md.) and can be used with the sequence-analysis programs blastp, blastn, blastx, tblastn and tblastx. BLAST™ can be accessed on the Internet at the NCBI website.

For comparisons of amino acid sequences of greater than about 30 amino acids, the “Blast 2 sequences” function of the BLAST™ program is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least about 45%, at least about 50%, at least about 60%, at least about 80%, at least about 85%, at least about 90%, or at least about 95% sequence identity.

Substantial similarity: A first nucleic acid is “substantially similar” to a second nucleic acid if, when optimally aligned (with appropriate nucleotide deletions or gap insertions) with the other nucleic acid (or its complementary strand), there is nucleotide sequence identity in at least about, for example, 50%, 75%, 80%, 85%, 90% or 95% of the nucleotide bases. Sequence similarity can be determined by comparing the nucleotide sequences of two nucleic acids using the BLAST™ sequence analysis software (blastn) available from The National Center for Biotechnology Information. Such comparisons can be made using the software set to default settings (expect=10, filter=default, descriptions=500 pairwise, alignments=500, alignment view=standard, gap existence cost=11, per residue existence=1, per residue gap cost=0.85). Similarly, a first polypeptide is substantially similar to a second polypeptide if they show sequence identity of at least about 75% to about 90% or greater when optimally aligned and compared using BLAST software (blastp) using default settings.

Taxoid: A “taxoid” is a chemical based on the Taxane ring structure, for example, as described in Kinston et al., Progress in the Chemistry of Organic Natural Products, Springer-Verlag, 1993.

Transacylase activity: Enzymes exhibiting transacylase activity are capable of transferring acyl groups, thus forming either esters or amides, by catalyzing reactions in which an acyl group that is linked to a carrier (acyl-carrier) is transferred to a reactant, thus forming an acyl group linked to the reactant (acyl-reactant).

Transacylases: Transacylases are enzymes that display transacylase activity. However, all transacylases do not recognize the same carriers and reactants. Therefore, transacylase enzyme-activity assays must utilize different substrates and reactants depending on the specificity of the particular transacylase enzyme. The assay described herein is a representative example of a transacylase activity assay, and similar assays can be used to test transacylase activity directed towards different substrates and reactants. Transacylases also are known by the name “acyltransferases.”

Transacylase nucleic acid: A nucleic acid encoding a polypeptide demonstrating transacylase activity.

Transformed: A “transformed” cell is a cell into which a nucleic acid molecule has been introduced by molecular biology techniques. As used herein, the term “transformation” encompasses all techniques by which a nucleic acid molecule might be introduced into such a cell, including transfection with a viral vector, transformation with a plasmid vector, and introduction of naked DNA by electroporation, lipofection, and particle gun acceleration. A “transformant” is an organism or microorganism that has been transformed.

Transgene. An exogenous nucleic acid supplied by a vector.

Transgenic. Of, pertaining to, or containing a nucleic acid, ORF, or other nucleic acid native to another species, microorganism, or virus. The term “transgenic” includes transient and permanent transformation, where the nucleic acid integrates into chromosomal DNA, including the germ line, or is maintained extrachromosomally. For example, and without limitation, plants transformed with a plasmid encoding transacylase disclosed herein (such the transacylases encoded by the TAX7 and TAX10 nucleic acids (SEQ ID NOS: 51 and 53)) maintained extrachromosomally are understood to be transgenic.

Vector: A “vector” is a nucleic acid molecule as introduced into a host cell, thereby producing a transformed host cell. A vector can include nucleic acid sequences, such as an origin of replication, that permit the vector to replicate in a host cell. A vector also can include one or more screenable markers, selectable markers, reporter genes, or other genetic elements.

Transacylase Protein and Nucleic acid Sequences

Transacylases and transacylase-specific nucleic acid sequences are disclosed, including utilizing the polymerase chain reaction (PCR) to identify and produce nucleic acid sequences encoding the transacylases. For example, PCR amplification of the transacylase sequences can be accomplished either by direct PCR from a plant cDNA library or by Reverse Transcriptase PCR (RT-PCR) using RNA extracted from plant cells as a template. Transacylase sequences can be amplified from plant genomic libraries, or plant genomic DNA. Methods and conditions for both direct PCR and RT-PCR are disclosed in, for example, Innis et al., 1999.

The selection of PCR primers is made according to the portions of the cDNA or other nucleic acid that are to be amplified. Primers can be chosen to amplify small segments of the cDNA, the open reading frame, the entire cDNA molecule, or the entire nucleic acid sequence. Variations in amplification conditions can be required to accomodate primers of differing lengths; such considerations are discussed, for example, in Innis et al., 1999; Sambrook et al., 2001; and Ausubel et al., 1987. By way of example (and without limitation), the cDNA molecules corresponding to additional transacylases can be amplified using primers directed towards regions of homology between the 5′ and 3′ ends of the TAX1 (SEQ ID NO: 27) and TAX2 (SEQ ID NO: 25) sequences. Examplary primers for such a reaction are:

(SEQ ID NO:42)

| | |
|---|---|
| primer 1: | 5′ CCT CAT CTT TCC CCC ATT GAT AAT 3′ |

(SEQ ID NO:43)

| | |
|---|---|
| primer 2: | 5′ AAA AAG AAA ATA ATT TTG CCA TGC AAG 3′ |

These primers are illustrative only and many different primers can be derived from the disclosed nucleic acid sequences. Re-sequencing of PCR products obtained by these amplification procedures can be used to facilitate confirmation of the amplified sequence and to provide information on natural variation between transacylase sequences. Oligonucleotides derived from the transacylase sequence can be used in such sequencing methods.

Oligonucleotides that are derived from the transacylase sequences are also encompassed within the scope of this disclosure. Such oligonucleotide primers comprise a sequence of at least 10 consecutive nucleotides of a transacylase sequence. In some embodiments, enhanced amplification specificity is provided by utilizing oligonucleotide primers comprising at least 15, 20, 25, 30, 35, 40, 45 or 50 consecutive nucleotides of these sequences.

Transacylases in other Plant Species

Orthologs of the transacylase genes are present in a number of other members of the Taxus genus. With the provision herein of the transacylase nucleic acid sequences, these orthologs encoding transacylases can be cloned from Taxus and other plant species. Orthologs of the disclosed transacylase genes have biological transacylase activity and are typically characterized by possession of at least 50% sequence identity counted over the fall length alignment with the amino acid sequence of the disclosed transacylase sequences using the NCBI Blast 2.0 (gapped blastp set to default parameters). Proteins with even greater similarity to the reference sequences will show increasing percentage identities when assessed by this method, such as at least 60%, at least 65%, at least 70%, at least 75%, at least 80%, at least 90%, or at least 95% sequence identity.

Both conventional hybridization and PCR amplification procedures can be utilized to clone sequences encoding transacylase orthologs. Common to both of these techniques is the hybridization of probes or primers that are derived from the transacylase nucleic acid sequences. Furthermore, the hybridization can occur in the context of Northern blots, Southern blots, or PCR.

Direct PCR amplification can be performed on cDNA or genomic libraries prepared from any of various plant species, or RT-PCR can be performed using mRNA extracted from plant cells of such species using standard methods. In particular embodiments, the PCR primers comprise at least 10 consecutive nucleotides of the transacylase sequences. Sequence differences between the transacylase nucleic acid sequence and the target nucleic acid to be amplified can result in lower amplification efficiencies. To compensate for this, longer PCR primers or lower annealing temperatures can be used during the amplification cycle. Where lower annealing temperatures are used, sequential rounds of amplification using nested primer pairs can enhance specificity.

A hybridization probe can be conjugated with a detectable label, such as a radioactive label. In some enbodiments, the hybridization probe is at least 10 nucleotides in length. Increasing the length of hybridization probes can enhance specificity. A labeled probe derived from the transacylase nucleic acid sequence can be hybridized to a plant cDNA or genomic library, and the hybridization signal then can be detected. The hybridizing colony or plaque (depending on the type of library used) then can be purified and the cloned sequence contained in that colony or plaque can be isolated and characterized.

Orthologs of the transacylases alternatively can be obtained by immunoscreening of an expression library. With the provision herein of the disclosed transacylase nucleic acid sequences, the enzymes can be expressed and purified in a heterologous expression system (e.g., E. coli) and used to raise antibodies (monoclonal or polyclonal) specific for transacylases. Antibodies also can be raised against synthetic peptides derived from the transacylase amino acid sequence described herein. Methods of raising antibodies are described in, for example, Harlow, Using Antibodies : A Laboratory Manual, Cold Spring Harbor Press, Cold Spring, N.Y., 1998; and Harlow and Lane, Antibodies, A Laboratory Manual, Cold Spring Harbor Press, Cold Spring, N.Y. 1988. Such antibodies then can be used to screen an expression cDNA library produced from a plant. This screening can identify the transacylase ortholog. The selected cDNAs can be confirmed by sequencing and by expressed enzyme activity assays.

Taxol® Transacylase Variants

Variants of the transacylase amino acid sequences (SEQ ID NOs: 2, 4, 6, 8, 10, 12, 14, 16, 18, 20, 22, 24, 26, 28, 45, 50, 52, 54, 56, and 58) and the corresponding cDNAs (SEQ ID NOs: 1, 3, 5, 7, 9, 11, 13, 15, 17, 19, 21, 23, 25, 27, 44, 49, 51, 53, 55, and 57) can be created.

Variant transacylases include proteins that differ in amino acid sequence from the transacylase sequences disclosed, but that retain biological transacylase activity. Manipulating the nucleotide sequence encoding the transacylase using standard procedures, such as site-directed mutagenesis or PCR, can produce such proteins. Simple modifications include the substitution of one or more amino acids for amino acids having similar biochemical properties. These so-called “conservative substitutions” are likely to have minimal impact on the activity of the resultant protein. Table 1 shows amino acids that can be substituted for an original amino acid in a protein and that are regarded as conservative substitutions.

TABLE 1

| Original | Conservative |
|---|---|
| Residue | Substitutions |
| ala | ser |
| arg | lys |
| asn | gln; his |
| asp | glu |
| cys | ser |
| gln | asn |
| glu | asp |
| gly | pro |
| his | asn; gln |
| ile | leu; val |
| leu | ile; val |
| lys | arg; gln; glu |
| met | leu; ile |
| phe | met; leu; tyr |
| ser | thr |
| thr | ser |
| trp | tyr |
| tyr | trp; phe |
| val | ile; leu |

More substantial changes in enzymatic function or other features can be obtained by selecting substitutions that are less conservative than those in Table 1, i.e., selecting residues that differ more significantly in their effect on maintaining: (a) the structure of the polypeptide backbone in the area of the substitution, for example, as a sheet or helical conformation; (b) the charge or hydrophobicity of the molecule at the target site; or (c) the bulk of the side chain. The substitutions likely produce the greatest changes in protein properties will be those in which: (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histidyl, is substituted for (or by) an electronegative residue, for example, glutamyl or aspartyl; or (d) a residue having a bulky side chain, for example, phenylalanine, is substituted for (or by) one not having a side chain, for example, glycine. The effects of these amino acid substitutions, deletions, or additions on transacylase derivatives can be assayed, for example, by analyzing the ability of the derivative proteins to catalyse the conversion of one Taxol® precursor or taxoid to another Taxol® precursor or taxoid.

Variant transacylase cDNAs (or nucleic acid sequences) can be produced by standard DNA mutagenesis techniques, for example, M13 primer mutagenesis. Details of such exemplary techniques are provided in Sambrook et al., 2001. By the use of such techniques, variants can be created that differ in minor ways from the transacylase cDNA or other nucliec acid sequences, yet that still encode a protein having biological transacylase activity. Nucleotide sequences that are derivatives of those disclosed herein and that differ from those disclosed nucleotide sequences by the deletion, addition, or substitution of nucleotides while still encoding a protein having biological transacylase activity are disclosed herein. For example, such variants can differ from the disclosed sequences by altering of the coding region to fit the codon usage bias of the particular organism into which the molecule is to be introduced.

Alternatively, the coding region can be altered by taking advantage of the degeneracy of the genetic code to alter the coding sequence in such a way that, while the nucleotide sequence is substantially altered, it nevertheless encodes a protein having an amino acid sequence identical or substantially similar to the disclosed transacylase amino acid sequences. For example, the fifteenth amino acid residue of the TAX2 (SEQ ID NO: 26) is alanine. This amino acid is encoded in the open reading frame (ORF) by the nucleotide codon triplet GCG. Because of the degeneracy of the genetic code, three other nucleotide codon triplets—GCA, GCC, and GCT—also code for alanine. Thus, the nucleotide sequence of the ORF can be changed at this position to any of these three codons without affecting the amino acid composition of the encoded protein or the characteristics of the protein. Based upon the degeneracy of the genetic code, variant DNA molecules can be derived from the cDNA and other nucleotide sequences disclosed herein, such as by using DNA mutagenesis techniques or by synthesis of DNA sequences. Thus, some embodiments include nucleic acid sequences that encode the transacylase protein but that vary from the disclosed nucleic acid sequences by virtue of the degeneracy of the genetic code.

Variants of the transacylase also can be defined in terms of their sequence identity with the disclosed transacylase amino acid and nucleic acid sequences. However, transacylases possessing biological transacylase activity share at least 60% sequence identity with the disclosed transacylase sequences. Nucleic acid sequences that encode such proteins can be readily determined by applying the genetic code to the amino acid sequence of the transacylase, and such nucleic acid molecules can be produced by assembling oligonucleotides corresponding to portions of the sequence.

Variants of the transacylases also can be identified by nucleic acid hybridization. Nucleic acid molecules that are derived from the transacylase cDNA and nucleic acid sequences include molecules that hybridize under various conditions to the disclosed Taxol® transacylase nucleic acid molecules, or fragments thereof. Generally, hybridization conditions are classified into categories, such as very high stringency, high stringency, and low stringency. The conditions for probes that are about 600 base pairs or more in length are provided below in three corresponding categories.

Very High Stringency (detects sequences that share 90%
sequence identity)

| | | | | |
|---|---|---|---|---|
| Hybridization in | 5x | SSC at | 65° C. | 16 hours |
| Wash twice in | 2x | SSC at | room temp. | 15 minutes each |
| Wash twice in | 2x | SSC at | 55° C. | 20 minutes each |

High Stringency (detects sequences that share 80%
sequence identity or greater)

| | | | | |
|---|---|---|---|---|
| Hybridization in | 5x | SSC at | 65° C. | 16 hours |
| Wash twice in | 2x | SSC at | room temp. | 20 minutes each |
| Wash once in | 2x | SSC at | 42° C. | 30 minutes each |

Low Stringency (detects sequences that share greater than 50%
sequence identity)

| | | | | |
|---|---|---|---|---|
| Hybridization in | 6x | SSC at | room temp. | 16 hours |
| Wash twice in | 2x | SSC at | room temp. | 20 minutes each |
| | | | (20-21° C.) | |

The sequences encoding the transacylases identified through hybridization also can incorporated into transformation vectors and introduced into host cells to produce a transacylase.

Introduction of a Transacylase into a Plant

After a cDNA or other nucleic acid encoding a protein involved in the determination of a particular plant characteristic has been isolated, that nucleic acid can be introduced into and expressed in a transgenic plant in order to modify that particular plant characteristic. The basic approach is to clone a cDNA encoding a transacylase into a transformation vector, such that the cDNA is operably linked to control sequences (for example, a promoter) directing expression of the cDNA in plant cells. The transformation vector is then introduced into plant cells (for example, by electroporation) and progeny plants containing the introduced cDNA are selected. All or part of the transformation vector can stably integrate into the genome of the plant cell. That part of the transformation vector that integrates into the plant cell and that contains the introduced nucleic acid, and associated sequences for controlling expression (the introduced “transgene”), can be referred to as the recombinant expression cassette.

Selection of progeny plants containing the introduced transgene (within the recombinant expresion cassette) can be made based upon detection of an altered phenotype. Such a phenotype can result directly from the oligomeric nucleic acid cloned into the transformation vector or can be manifested as enhanced resistance to a chemical agent (such as an antibiotic) as a result of the inclusion of a dominant selectable marker nucleic acid incorporated into the transformation vector.

Successful examples of the modification of plant characteristics by transformation with cloned nucleic acids are replete in the technical and scientific literature. Selected examples, which serve to illustrate transformation of plants, include:

U.S. Pat. No. 5,571,706 (“Plant Virus Resistance Gene and Methods”)

U.S. Pat. No. 5,677,175 (“Plant Pathogen Induced Proteins”)

U.S. Pat. No. 5,510,471 (“Chimeric Gene for the Transformation of Plants”)

U.S. Pat. No. 5,750,386 (“Pathogen-Resistant Transgenic Plants”)

U.S. Pat. No. 5,597,945 (“Plants Genetically Enhanced for Disease Resistance”)

U.S. Pat. No. 5,589,615 (“Process for the Production of Transgenic Plants with Increased Nutritional Value Via the Expression of Modified 2S Storage Albumins”)

U.S. Pat. No. 5,750,871 (“Transformation and Foreign Gene Expression in Brassica Species”)

U.S. Pat. No. 5,268,526 (“Overexpression of Phytochrome in Transgenic Plants”)

U.S. Pat. No. 5,262,316 (“Genetically Transformed Pepper Plants and Methods for their Production”)

U.S. Pat. No. 5,569,831 (“Transgenic Tomato Plants with Altered Polygalacturonase Isoforms”)

These exemplary references include descriptions of transformation vector selection, transformation techniques, and the assembly of constructs designed to over-express the introduced nucleic acid sequence. Thus, nucleic acids and polypeptides, or homologous or derivative forms of these molecules, can be introduced into plants in order to produce plants having enhanced transacylase activity. For example, the expression of one or more transacylases in plants can give rise to plants having increased production of Taxol® and related compounds.

Vector Construction, Choice of Promoters

A number of recombinant vectors suitable for stable transfection of plant cells or for the establishment of transgenic plants are described in, for example, Weissbach and Weissbach, Methods for Plant Molecular Biology, Academic Press, 1989; and Gelvin et al., Plant and Molecular Biology Manual, Kluwer Academic Publishers, 1990. Typically, plant-transformation vectors include one or more cloned nucleic acids (for example, cDNA) under the transcriptional control of 5′- and 3′-regulatory sequences and a dominant selectable marker. Such plant transformation vectors also can contain a promoter regulatory region (for example, a regulatory region controlling inducible or constitutive, environmentally or developmentally regulated, or cell- or tissue-specific expression), a transcription initiation start site, a ribosome binding site, an RNA processing signal, a transcription termination site, and/or a polyadenylation signal.

Examples of constitutive plant promoters useful for expressing a nucleic acid include (but are not limited to): the cauliflower mosaic virus (CaMV) 35S promoter, which confers constitutive, high-level expression in most plant tissues (see, e.g., Odel et al., Nature 313:810, 1985; Dekeyser et al., Plant Cell 2:591, 1990; Terada and Shimamoto, Mol. Gen. Genet. 220:389, 1990; and Benfey and Chua, Science 250:959-966, 1990); the nopaline synthase promoter (An et al., Plant Physiol. 88:547, 1988); and the octopine synthase promoter (Fromm et al., Plant Cell 1:977, 1989). Agrobacterium-mediated transformation of Taxus species has been accomplished, and the resulting callus cultures have been shown to produce Taxol® (Han et al., Plant Science 95: 187-196, 1994). Therefore, incorporation of one or more of the disclosed transacylases under the influence of a strong promoter (such as CaMV promoter) increases production yields of Taxol® and related taxoids in such transformed cells.

A variety of plant promoters that are regulated in response to environmental, hormonal, chemical, and/or developmental signals also can be used for expression of the nucleic acids in plant cells, including promoters regulated by: (a) heat (Callis et al., Plant Physiol. 88:965, 1988; Ainley, et al., Plant Mol. Biol. 22:13-23, 1993; and Gilmartin et al., Plant Cell 4:839-949, 1992); (b) light (e.g., the pea rbcS-3A promoter, Kuhlemeier et al., Plant Cell 1:471, 1989, and the maize rbcS promoter, Schaffner and Sheen, Plant Cell 3:997, 1991); (c) hormones, such as abscisic acid (Marcotte et al., Plant Cell 1:969, 1989); (d) wounding (see, e.g., Siebertz et al., Plant Cell 1:961-68, 1989); and (e) chemicals such as methyl jasmonate or salicylic acid (Gatz et al., Ann. Rev. Plant PhysioL Plant Mol. Biol. 48:9-108, 1997).

Alternatively, tissue-specific (root, leaf, flower, and seed, for example) promoters can be fused to the coding sequence to obtain a particular expression in respective organs. (See, e.g., Carpenter et al., Plant Cell 4:557-571, 1992; Denis et al., Plant Physiol. 101:1295-1304, 1993; Opperman et al., Science 263:221-223, 1993; Stockhause et al., Plant Cell 9:479-489, 1997; Roshal et al., EMBO J. 6:1155, 1987; Schermthaner et al., EMBO J. 7:1249, 1988; and Bustos et al., Plant Cell 1:839, 1989).

Alternatively, native transacylase nucleic acid promoters, or fragments thereof, can be utilized. The determination of whether a particular region of a transacylase neucleotide sequence confers effective promoter activity can be ascertained, such as by operably linking the selected sequence region to a transacylase cDNA (in conjunction with suitable 3′ regulatory region, such as the NOS 3′ regulatory region as disclosed herein) and determining whether the transacylase is expressed.

Plant-transformation vectors also can include RNA-processing signals, for example, introns, that may be positioned upstream or downstream of the ORF sequence in the transgene. In addition, the expression vectors also can include additional regulatory sequences from the 3′-untranslated region of plant genes, for example, a 3′-terminator region to increase mRNA stability of the mRNA, such as the PI-II terminator region of potato or the octopine or nopaline synthase (NOS) 3′-terminator regions. The native transacylase nucleic acid 3′-regulatory sequence also can be employed.

Finally, plant-transformation vectors also can include dominant selectable markers to facilitate selection of transgenic plants. Such markers include those nucleic acids encoding antibiotic-resistance (for example, resistance to hygromycin, kanamycin, bleomycin, G418, streptomycin or spectinomycin) and herbicide-resistance (for example, phosphinothricin acetyltransacylase).

Arrangement of Taxol® Transacylase Sequence in a Vector

The particular arrangement of transacylase nucleotide sequence in the transformation vector is selected according to the type of expression of the sequence that is desired.

In some embodiments, enhanced transacylase activity is desired, and the transacylase ORF is operably linked to a constitutive high-level promoter, such as the CaMV 35S promoter. Enhanced transacylase activity also can be achieved by introducing into a plant a transformation vector containing a variant form of the transacylase nucleic acid, for example a form that varies from the exact nucleotide sequence of a transacylase ORF, but that encodes a protein retaining transacylase biological activity.

Transformation and Regeneration Techniques

Transformation and regeneration of both monocotyledonous and dicotyledonous plant cells are now routine, and the appropriate transformation technique can be determined by a practitioner. The choice of method can vary with the type of plant to be transformed. The suitability of particular methods for given plant types can be ascertained. Suitable methods include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumefaciens (AT) mediated transformation. Typical procedures for transforming and regenerating plants are described in the patent documents referenced above.

Selection of Transformed Plants

Following transformation and regeneration of plants (for example, plants within the Taxus genus) with the transformation vector, transformed plants can be selected using a dominant selectable marker incorporated into the transformation vector. For example, if the marker confers antibiotic resistance on the seedlings of transformed plants, selection of transformants can be accomplished by exposing the seedlings to appropriate concentrations of the antibiotic.

After transformed plants are selected and grown to maturity, they can be assayed to assess production levels of Taxol® and related compounds, such as (and without limitation), by using the assay methods described herein.

Production of Recombinant Taxol® Transacylase in Heterologous Expression Systems

Various yeast strains and yeast-derived vectors can be used for the expression of heterologous proteins, for example, the Pichia pastoris expression systems manufactured by the Invitrogen Corp. (Carlsbad, Calif.). Such systems include suitable Pichia pastoris strains, vectors, reagents, transformants, sequencing primers, and media. Available strains include KM71H (a prototrophic strain), SMD1168H (a prototrophic strain), and SMD1168 (a pep4 mutant strain) (Invitrogen Product Catalogue, 1998, Invitrogen Corp., Carlsbad Calif.).

Non-yeast eukaryotic vectors can be used with equal facility for expression of proteins encoded by the disclosed nucleic acids. Mammalian vector/host cell systems containing genetic and cellular control elements capable of carrying out transcription, translation, and post-translational modification can be utilized. Examples of such systems include, but are not limited to, the baculovirus system, the ecdysone-inducible expression system that uses regulatory elements from Drosophila melanogaster to allow control of nucleic acid expression, and the sindbis viral-expression system that allows high-level expression in a variety of mammalian cell lines, all of which are available from the Invitrogen Corp., Carlsbad, Calif.

The cloned expression vector encoding one or more transacylases can be transformed into any of various cell types for expression of the cloned nucleotide. Many different types of cells can be used to express modified nucleic acid molecules, including (but not limited to) the eukaryotic and prokaryotic cells described herein. Examples include cells of yeasts, fungi, bacteria, insects, mammals, and plants, including transformed and non-transformed cells. For instance, common mammalian cells that could be used include HeLa cells, SW-527 cells (ATCC deposit #7940), WISH cells (ATCC deposit #CCL-25), Daudi cells (ATCC deposit #CCL-213), Mandin-Darby bovine kidney cells (ATCC deposit #CCL-22) and Chinese hamster ovary (CHO) cells (ATCC deposit #CRL-2092). Common yeast cells include Pichia pastoris (ATCC deposit #201178) and Saccharomyces cerevisiae (ATCC deposit #46024). Bacterial cells include E. coli JM109 (ATCC deposit #53323). Insect cells include cells from Drosophila melanogaster (ATCC deposit #CRL-10191), the cotton bollworm (ATCC deposit #CRL-9281), and Trichoplusia ni egg cell homoflagellates. Fish cells that can be used include those from rainbow trout (ATCC deposit #CLL-55), salmon (ATCC deposit #CRL-1681), and zebrafish (ATCC deposit #CRL-2147). Amphibian cells that can be used include those of the bullfrog, Rana castebelana (ATCC deposit #CLL-41). Reptile cells that can be used include those from Russell's viper (ATCC deposit #CCL-140). Plant cells that could be used include Chlamydomonas cells (ATCC deposit #30485), Arabidopsis cells (ATCC deposit #54069) and tomato plant cells (ATCC deposit #54003). Many of these cell types are commonly used and are available from the American Type Culture Collection as well as from commercial suppliers, such as the Pharmacia Corp. (Uppsala, Sweden), and the Invitrogen Corp.

Expressed protein can be accumulated within a cell or can be secreted from the cell. Such expressed protein can then be collected and purified. This protein can then be characterized for activity and stability and utilized in different ways, such as being used to practice the methods diclosed herein.

Creation of Transacylase-Specific Binding Agents

Antibodies to transacylase proteins, and fragments thereof, can be used for purification of those proteins. Antibody-based binding agents to these proteins can be produced using the sequences discussed herein.

Monoclonal or polyclonal antibodies to antigens of the transacylases, portions of the transacylases, or variants thereof can be produced. Antibodies raised against epitopes on antigens can specifically detect the corresponding antigen. That is, antibodies raised against the transacylases selectively or specifically recognize and bind the transacylases, yet do not substantially recognize or bind to other proteins. The determination that an antibody binds to an antigen can be made by an immunoassay methods, for example, Western blotting or ELISA.

As just one non-limiting example, to determine that a given antibody preparation (such as a preparation produced in a mouse against TAX1) specifically detects the transacylase by Western blotting, total cellular protein is extracted from transfected cells, transformed cells, or from wild-type cells and electrophoresed on an SDS-polyacrylamide gel. The proteins are then transferred to a membrane (for example, nitrocellulose) by Western blotting, and the antibody preparation is incubated with the membrane. After washing the membrane to remove non-specifically bound antibodies, the presence of specifically bound antibodies is detected by the use of, for example, an anti-mouse antibody conjugated to an enzyme such as alkaline phosphatase; application of 5-bromo-4-chloro-3-indolyl phosphate/nitro blue tetrazolium results in the production of a densely blue-colored compound by immuno-localized alkaline phosphatase.

Antibodies that selectively or specifically recognize and bind to a transacylase can be shown to bind substantially only the transacylase band (having a position on the gel consistent with that of the molecular weight of the transacylase). Non-specific binding of the antibody to other proteins can occur and may be detectable as a weaker signal on the Western blot (which can be quantified by automated radiography). The non-specific nature of this binding can be assessed by the weak signal obtained on the Western blot relative to the strong primary signal arising from the specific anti-transacylase binding.

An agent, such as an antibody, that “selectively” binds to a particular target exhibits some preference for its target over other similar targets. Some antibodies (both monoclonal and polyclonal) can discriminate between closely related epitopes and also can distinguish between different antigens, if those antigens express epitopes that are not shared by other antigens. As one non-limiting example, an agent that binds transacylases from plants, but not transacylases obtained from mammals or bacteria, is an agent that selectively binds transacylases from plants. As another non-limiting example, an agent selectively binds to Taxus transacylases if that agent recognizes and binds to transacylases obtained from plants of the Taxus genus but does not recognize or bind to transacylases from other plants.

An agent that “specifically” binds to a particular target binds substantially only to a defined target. As used herein, a specific binding agent includes both monoclonal and polyclonal antibodies. As one non-limiting example, an agent that binds to a particular transacylase (such as the transacylase encoded by the TAX7 amino acid sequence (SEQ ID NO: 52)) but not to other transacylases (such as other Taxus transacylases) is an agent that specifically binds to that particular transacylase.

Antibodies that specifically bind to transacylases belong to a class of molecules that are referred to herein as “specific binding agents.” Specific binding agents that are capable of specifically binding to a transacylase can include polyclonal antibodies, monoclonal antibodies, and fragments of monoclonal antibodies such as Fab, F(ab′)₂ and Fv fragments, as well as any other agent capable of specifically binding to one or more epitopes on the transacylases.

Substantially pure transacylases suitable for use as an immunogen can be isolated from transfected cells, transformed cells, or from wild-type cells. Concentration of protein in the final preparation can be adjusted, for example, by concentration on an Amicon filter device, to the level of a few micrograms per milliliter. Alternatively, peptide fragments of a transacylase can be utilized as immunogens. Such fragments can be chemically synthesized, or can be obtained by cleavage of the transacylase polypeptide and purification of the desired peptide fragments. Peptides as short as three or four amino acids in length can be immunogenic when presented to an immune system in the context of a Major Histocompatibility Complex (MHC) molecule, such as MHC class I or MHC class II. Accordingly, peptides comprising at least 3, 4, 5, 6 or more consecutive amino acids of the disclosed transacylase amino acid sequences can be employed as immunogens for producing antibodies.

Some naturally occurring epitopes on proteins comprise amino acid residues that are not adjacently arranged in the peptide when the peptide sequence is viewed as a linear molecule. Therefore, some embodiments utilize longer peptide fragments from the transacylase amino acid sequences for producing antibodies. For example, peptides comprising at least 10, 15, 20, 25, or 30 consecutive amino acid residues of the amino acid sequence can be employed. Monoclonal or polyclonal antibodies to the intact transacylase, or peptide fragments thereof can be prepared as described herein.

Monoclonal Antibody Production by Hybridoma Fusion

A monoclonal antibody to any of the epitopes of the transacylase proteins described herein can be prepared such as by using murine hybridomas according to the classic method of Kohler & Milstein, Nature 256:495, 1975, or a similar method. Briefly, a mouse is *44 repetitively inoculated with a few micrograms of the selected protein over a period of a few weeks. The mouse is then sacrificed, and the antibody-producing cells of the spleen are isolated. The spleen cells are then fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA as originally described by Engvall, Enzymol. 70:419, 1980. Selected positive clones can be expanded and their monoclonal antibody product harvested for use. Detailed procedures for monoclonal antibody production are described in, for example, Harlow & Lane, Antibodies, A Laboratory Manual, Cold Spring Harbor Laboratory, N.Y., 1988.

Polyclonal Antibody Production by Immunization

Polyclonal antiserum containing antibodies to heterogenous epitopes of a single protein can be prepared by immunizing suitable animals with the expressed protein, which can be unmodified or modified to enhance immunogenicity. Effective polyclonal antibody production is affected by many factors related both to the antigen and the host species. For example, small molecules tend to be less immunogenic than other molecules and can require the use of carriers and an adjuvant. Also, host animals can vary in response to thesite of inoculation and dose; for example, both inadequate or excessive doses of antigen can result in low-titer antisera. Small doses (ng level) of antigen administered at multiple intradermal sites appear to be most reliable. An effective immunization protocol producing polyclonal antibodies for rabbits can be found in Vaitukaitis et al., J. Clin. Endocrinol. Metab. 33:988-991, 1971.

Booster injections can be given at regular intervals, and antiserum harvested when the antibody titer thereof, as determined semi-quantitatively, for example, by double immunodiffusion in agar against known concentrations of the antigen, begins to fall. See, for example, Ouchterlony et al., Handbook of Experimental Immunology, Wier, D. (ed.), Chapter 19, Blackwell, 1973. A plateau concentration of antibody can be in the range of 0.1 to 0.2 mg/mL of serum (about 12 μM). Affinity of the antisera for the antigen can be determined by preparing competitive binding curves using conventional methods.

Antibodies Raised by Injection of cDNA

Antibodies also can be raised against the disclosed transacylases by subcutaneous injection of a DNA vector that expresses the enzymes in laboratory animals, such as mice. Delivery of the recombinant vector into the animals can be achieved using a hand-held form of the Biolistic system (Sanford et al., Particulate Sci Technol. 5:27-37, 1987, as described by Tang et al., Nature (London) 356:153-154, 1992). Expression vectors suitable for this purpose can include those that express the cDNA of the enzyme under the transcriptional control of either the human β-actin promoter or the cytomegalovirus (CMV) promoter. Methods of administering naked DNA to animals in a manner resulting in expression of the DNA in the body of the animal include those described in, for example, U.S. Pat. Nos. 5,620,896 (“DNA Vaccines Against Rotavirus Infections”); 5,643,578 (“Immunization by Inoculation of DNA Transcription Unit”); and 5,593,972 (“Genetic Immunization”), and references cited therein.

Antibody Fragments

Antibody fragments can be used in place of whole antibodies and can be readily expressed in prokaryotic host cells. Methods of making and using immunologically effective portions of monoclonal antibodies, also referred to as “antibody fragments,” include those described in Better & Horowitz, Methods Enzymol. 178:476-496, 1989; Glockshuber et al. Biochemistry 29:1362-1367, 1990; and U.S. Pat. Nos. 5,648,237 (“Expression of Functional Antibody Fragments”); No. 4,946,778 (“Single Polypeptide Chain Binding Molecules”); and No. 5,455,030 (“Immunotherapy Using Single Chain Polypeptide Binding Molecules”), and references cited therein.

Taxol® Production in vivo

The creation of recombinant vectors and transgenic organisms expressing the vectors can be used to produce transacylases. These vectors can be used to decrease transacylase production, or to increase transacylase production. A decrease in transacylase production can result from the inclusion of an antisense sequence or a catalytic nucleic acid sequence that targets the transacylase encoding nucleic acid sequence. Conversely, increased production of transacylase can be achieved by including at least one additional transacylase encoding sequence in the vector. These vectors can then be introduced into a host cell, thereby altering transacylase production. In the case of increased production, the resulting transacylase can be used in in vitro systems, as well as in vivo for increased production of Taxol®, other taxoids, intermediates of the Taxol® biosynthetic pathway, and other products.

Increased production of Taxol® and related taxoids in vivo can be accomplished by transforming a host cell, such as one derived from a plant of the Taxus genus, with a vector containing one or more nucleic acid sequences encoding one or more transacylases. Furthermore, the heterologous or homologous transacylase sequences can be placed under the control of a constitutive promoter or an inducible promoter. Such transformation can lead to the increased production of transacylase, thus eliminating any rate-limiting effect on Taxol® production caused by the expression and/or activity level of the transacylase.

Taxol® Production in vitro

Currently, Taxol® is produced by a semisynthetic method described in Holton et al., Taxol ™: Science and Applications, CRC Press, Boca Raton, 97-121, 1995. This method involves extracting 10-deacetyl-baccatin III, or baccatin III, intermediates in the Taxol® biosynthetic pathway and then finishing the production of Taxol® using synthetic techniques. As more enzymes are identified in the Taxol® biosynthetic pathway, it may become possible to completely synthesize Taxol® in vitro, or at least increase the number of steps that can be performed in vitro. Hence, the transacylases disclosed herein can be used to facilitate the production of Taxol® and related taxoids in synthetic or semi-synthetic methods. Accordingly, transgenic organisms can produce increased levels of Taxol®, and can also produce increased levels of important intermediates in the Taxol® biosynthetic pathway, such as 10-deacetylbaccatin III, baccatin III, and β-phenylalanoyl baccatin III.

Other Transacylases of the Taxol® Pathway

The protocols described in Example 1 below yielded twelve related amplicons. Initial use of the first and second amplicons as probes for screening the cDNA library allowed for the isolation and characterization of taxadienol 5-O-acetyl transacylase. In addition to this first confirmed taxadienol 5-O-acetyl transacylase (TAX1), there are at least four additional transacylation steps in the Taxol® biosynthetic pathway represented by the 2-debenzoyl baccatin III-2-O-benzoyl transacylase, the 10-deacetylbaccatin III-10-O-acetyl transacylase, the baccatin III-13-O-phenylisoseryl transacylase, and the debenzoyltaxol-N-benzoyl transacylase. The close relationship between the nucleic acid sequences of the twelve amplicons indicates that the remaining amplicon sequences represent partial nucleic acid sequences of the other transacylases in the Taxol® pathway. Hence, the protocols described herein enable full-length versions of these Taxol® transacylases to be obtained. The following discussion relating to Taxol® transacylases refers to taxadienol 5-O-acetyl transacylase, as well as the remaining transacylases of the Taxol® pathway. Furthermore, transacylases can be tested for enzymatic activity using functional assays with the appropriate taxoid substrates, for example, the assay for taxoid C10 transacylase described in Menhard and Zenk, Phytochemistry 50:763-774, 1999.

### EXAMPLES

The following examples are provided to illustrate particular features of certain embodiments, but the scope of the claims should not be limited to those features exemplified.

### Example 1

### Characterization of acetyl CoA:taxa-4(20),11(12)-dien-5α-ol O-acetyl transacylase

Enzyme Purification and Library Construction

Biochemical studies have indicated that the third specific intermediate of the Taxol® biosynthesis pathway is taxa-4(20),11(12)-dien-5α-yl acetate, because this metabolite serves as a precursor of a series of polyhydroxy taxanes en route to the end-product (Hezari and Croteau, Planta Medica 63:291-295, 1997). The responsible enzyme, taxadienol acetyl transacylase, that converts taxadienol to the C5-acetate ester is, thus, an important candidate for cDNA isolation for the purpose of overexpression in relevant producing organisms to increase Taxol® yield (Walker et al., Arch. Biochem. Biophys. 364:273-279, 1999).

This enzyme has been partially purified and characterized with respect to reaction parameters (Walker et al., Arch. Biochem. Biophys. 364:273-279, 1999); however, the published fractionation protocol does not yield a pure protein suitable for amino acid microsequencing that is required for an attempt at reverse genetic cloning of the nucleic acid. Additionally, the partially characterized polypeptide has no homologs or orthologs (for example, other terpenoid or isoprenoid O-acetyl transacylases) in the databases that would permit similarity-based cloning approaches.

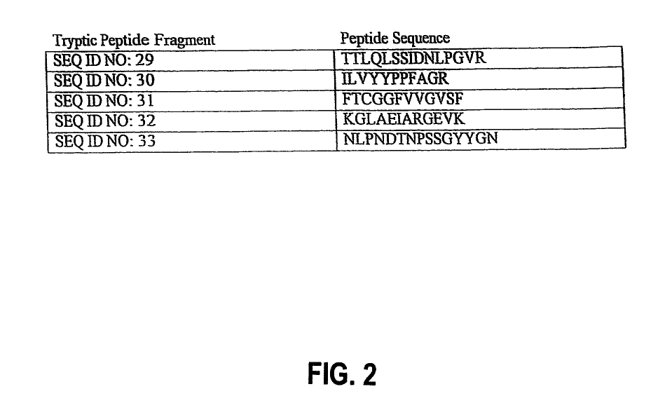

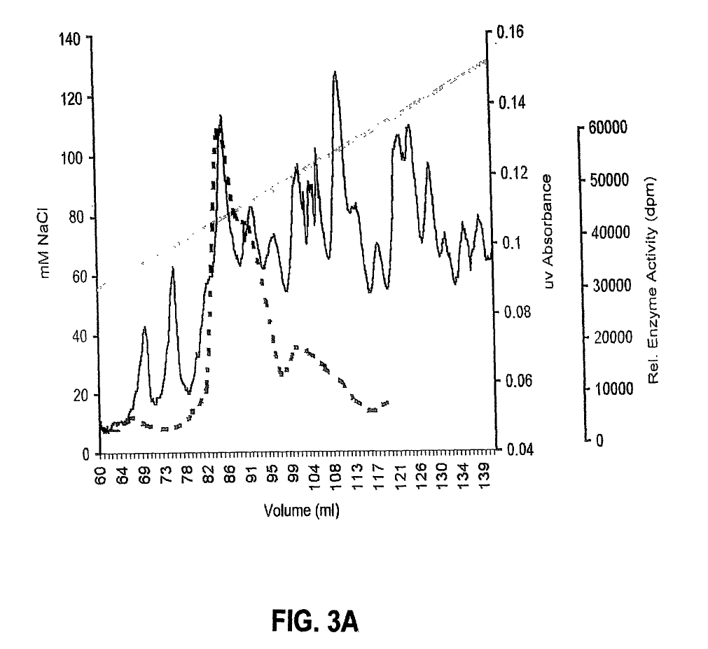

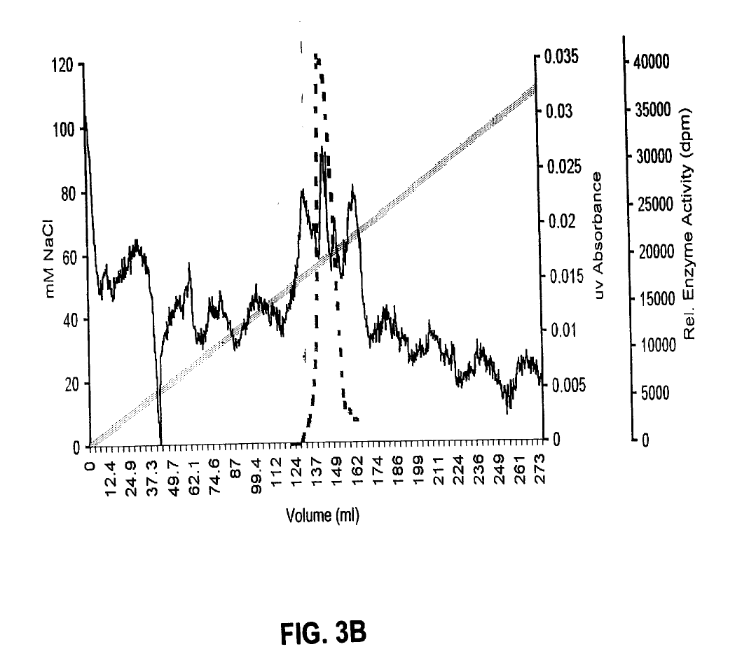

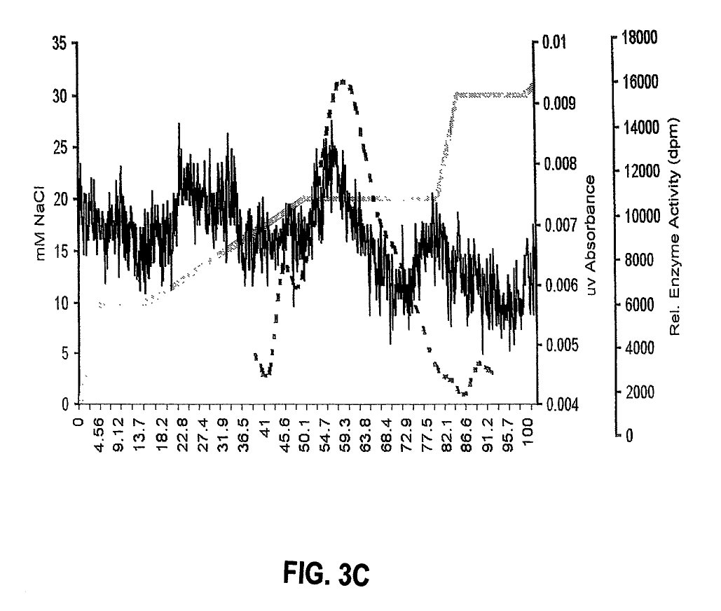

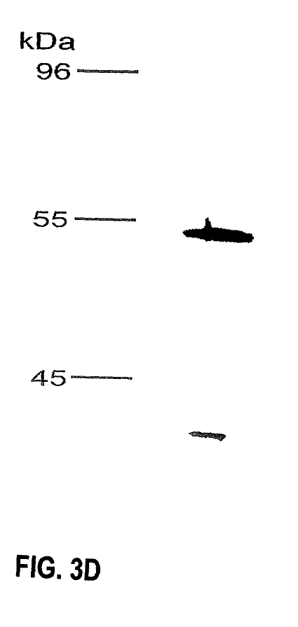

Using methyl jasmonate-induced Taxus canadensis cells as an enriched enzyme source, a new isolation and purification protocol (see FIGS. 3A-3C, and protocol described herein) was developed to efficiently yield homogeneous protein for microsequencing. Although the protein was N-blocked and failed to yield peptides that could be internally sequenced by V8 (endoproteinase Glu-C, Roche Molecular Biochemical, Nutley, N.J.) proteolysis or cyanogen bromide (CNBr) cleavage, treatment with endolysC (endoproteinase Lys-C, Roche Molecular Biochemical, Nutley, N.J.) and trypsin yielded a mixture of peptides. Five of these could be separated by high-performance liquid chromatography (HPLC) and verified by mass spectrometry (MS), and yielded sequence information useful for a cloning effort (FIG. 2).

For cDNA library construction, a stable, methyl jasmonate-inducible T. cuspidata suspension cell line was chosen for mRNA isolation because the production of Taxol® was highly inducible in this system (which permits the preparation of a suitable subtractive library, if necessary). The mixing of experimental protocols as used with different Taxus species is not a significant limitation, since all Taxus species are known to be very closely related and are considered by several taxonomists to represent geographic variants of the basic species T. baccata (Bolsinger and Jaramillo, Silvics of Forest Trees of North America (revised), Pacific Northwest Research Station, USDA, p. 17, Portland, Oreg., 1990; and Voliotis, Isr. J. Botany. 35:47-52, 1986). Thus, the genes encoding geranylgeranyl diphosphate synthase and taxadiene synthase (early steps of Taxol® biosynthesis) from T. canadensis and T. cuspidataevidence only very minor sequence differences. Hence, a method was developed for the isolation of high-quality mRNA from Taxus cells (Qiagen, Valencia, Calif.) and this material was employed for cDNA library construction using a commercial kit, which is available from Stratagene (La Jolla, Calif.).

Reverse Genetic Cloning

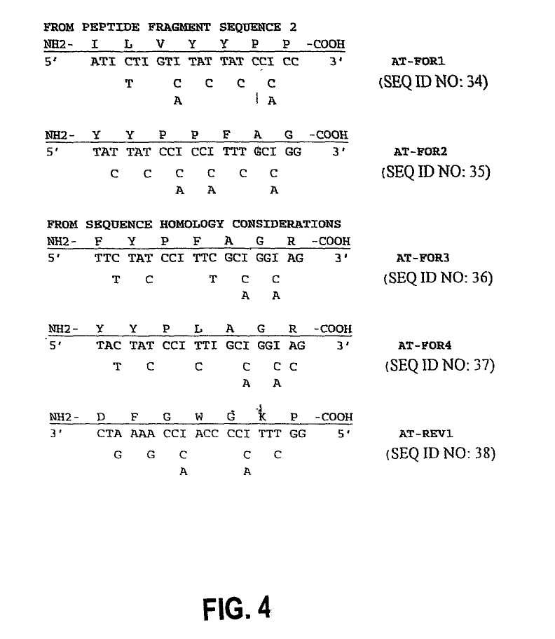

Of the five tryptic peptides that were sequenced (FIG. 2), peptide SEQ ID NOs: 30, 31, and 33 were found to exhibit some similarity to the sequences of the only two other plant acetyl transacylases that have been documented, namely, deacetylvindoline O-acetyl transacylase involved in indole alkaloid biosynthesis (St. Pierre et al., Plant J. 14:703-713, 1998) and benzyl alcohol O-acetyl transacylase involved in the biosynthesis of aromatic esters of floral scent (Dudareva et al., Plant J. 14:297-304, 1998). Lesser resemblance was found to a putative aromatic O-benzoyl transacylase of plant origin (Yang et al., Plant Mol. Biol. 35:777-789, 1997). Of the five peptide sequences (FIG. 2), SEQ ID NO: 30 was most suitable for primer design based on codon degeneracy considerations, and two such forward degenerate primers, AT-FOR1 (SEQ ID NO: 34) and AT-FOR2 (SEQ ID NO: 35), were synthesized (FIG. 4). A search of the database with the tryptic peptide ILVYYPPFAGR (SEQ ID NO: 30) revealed two possible variants of this sequence among several polypeptide entries of known and unknown function (these entries are listed in Table 2). Consideration of these distantly related sequences allowed the design of two additional forward degenerate primers (AT-FOR3 (SEQ ID NO: 36) and AT-FOR4 (SEQ ID NO: 37)), and permitted identification of a distal consensus sequence from which a degenerate reverse primer (AT-REV1 (SEQ ID NO: 38)) was designed (FIG. 4). An alignment of the Taxus sequences with the extant database sequence entries of Table 2 illustrates the lack of significant homology between the Taxus sequences and any previously described polypeptides.

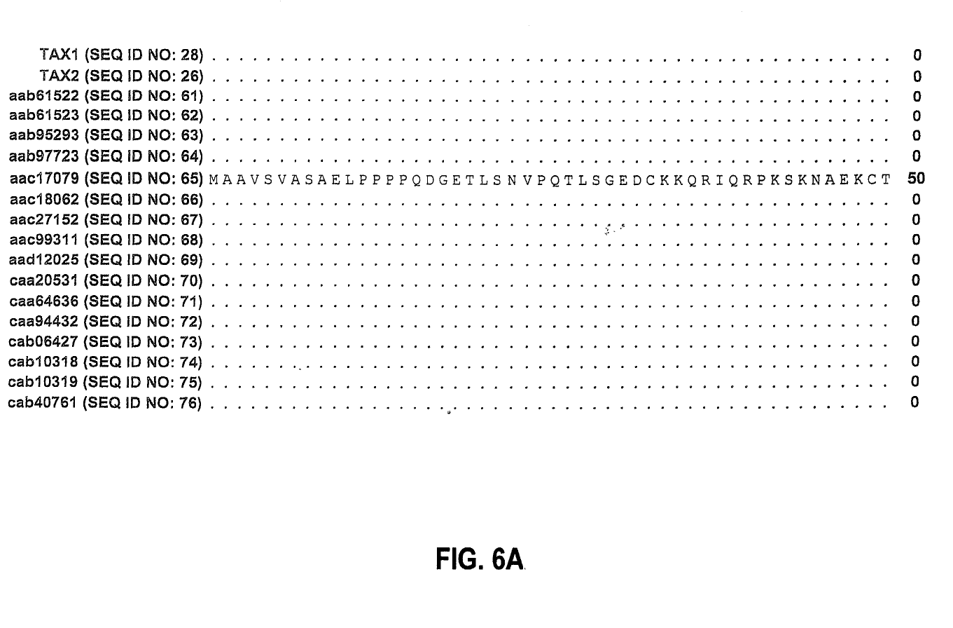

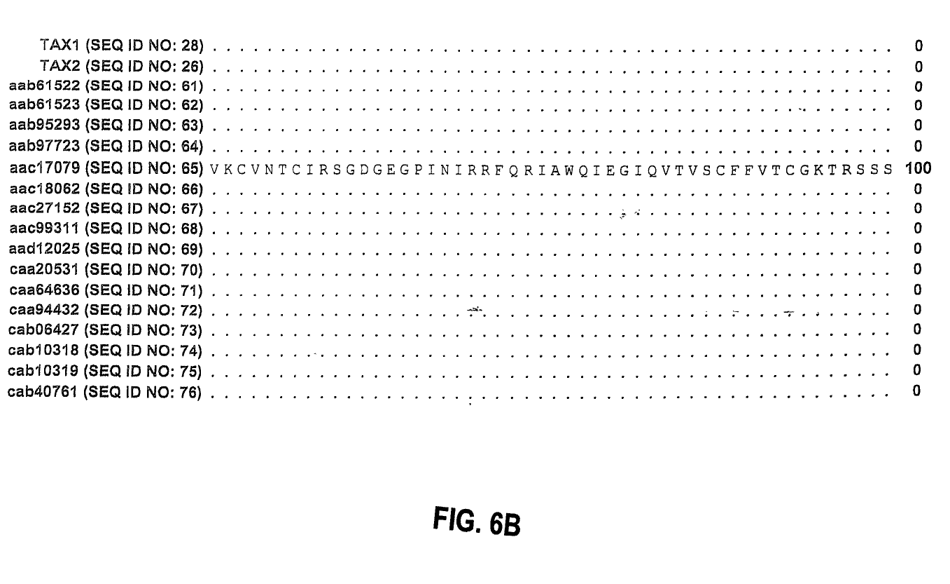

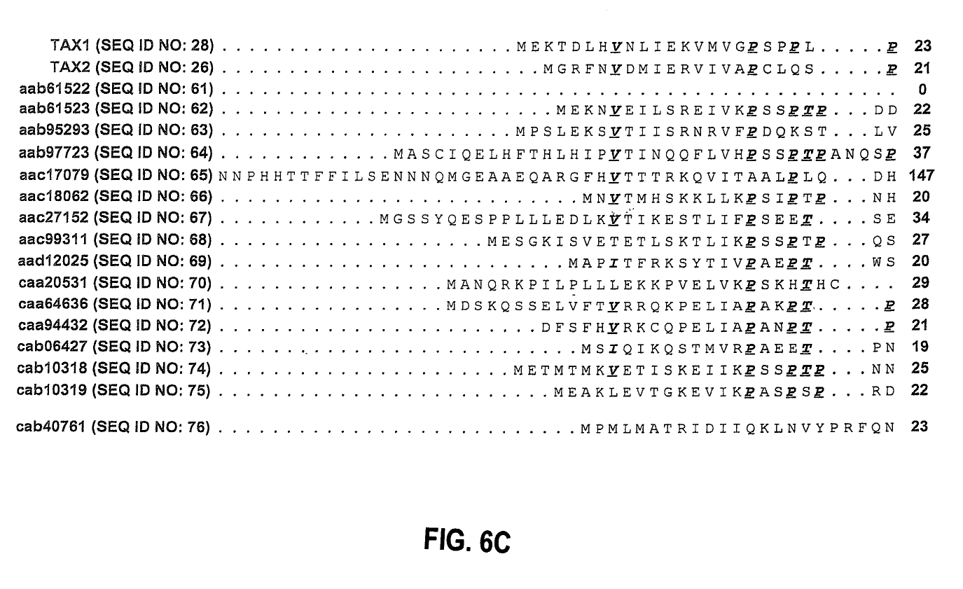

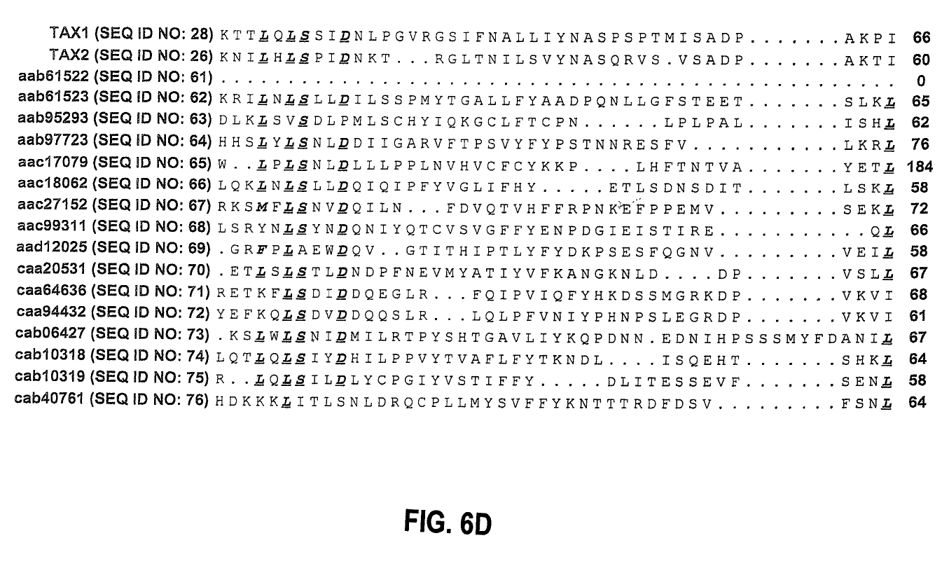

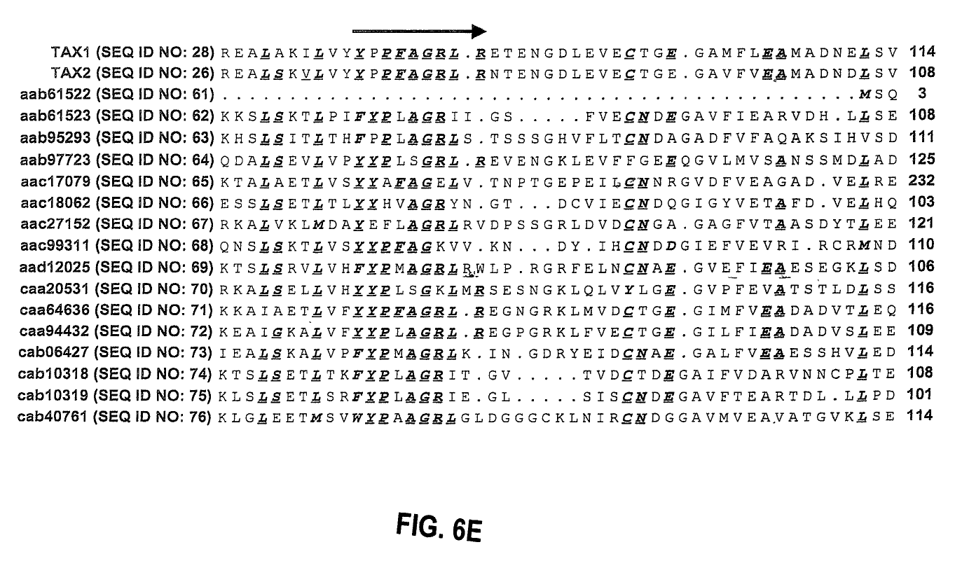

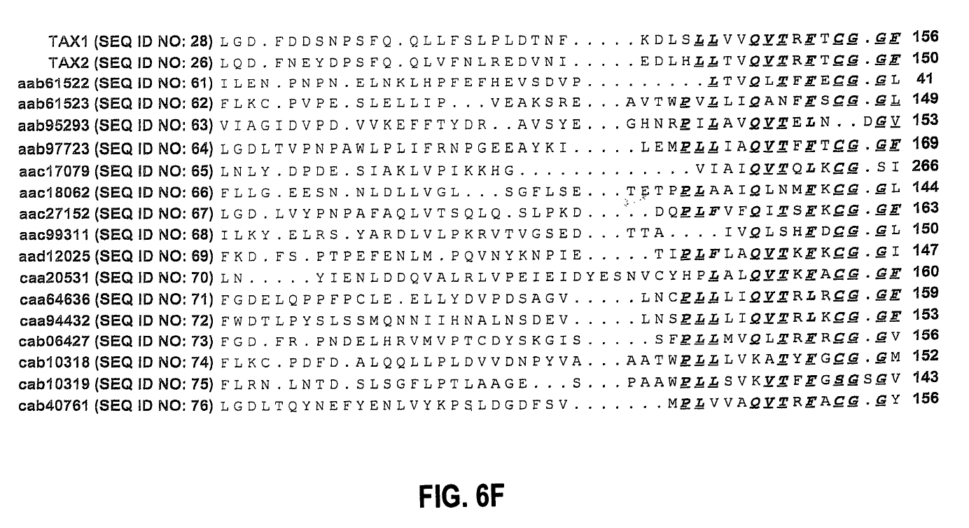

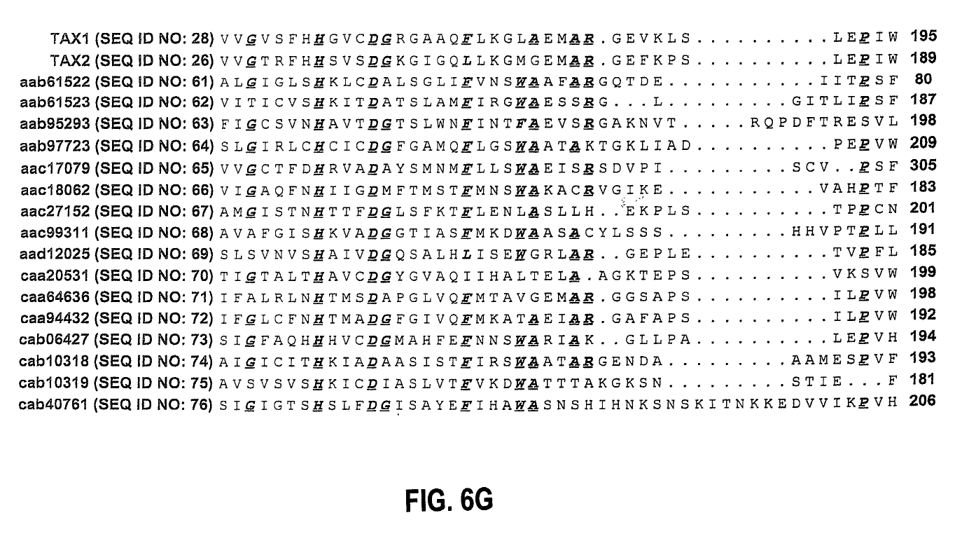

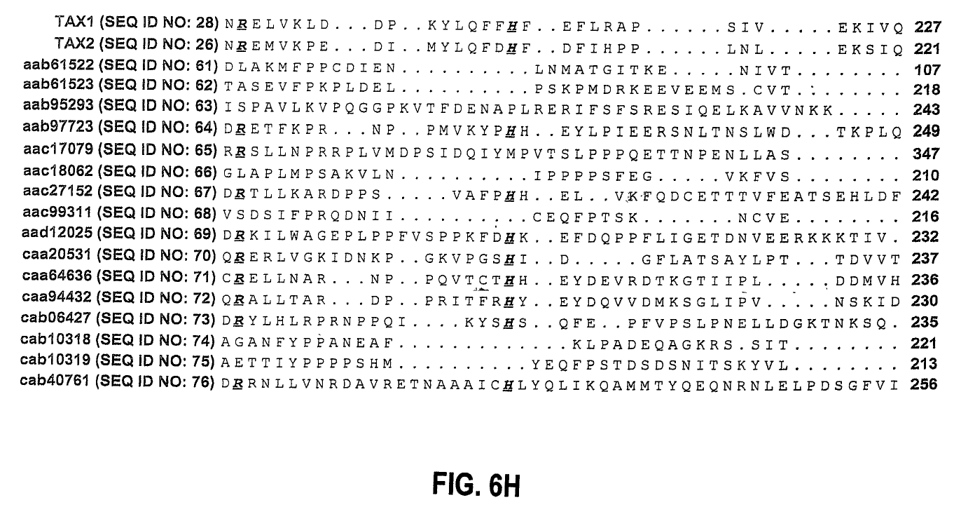

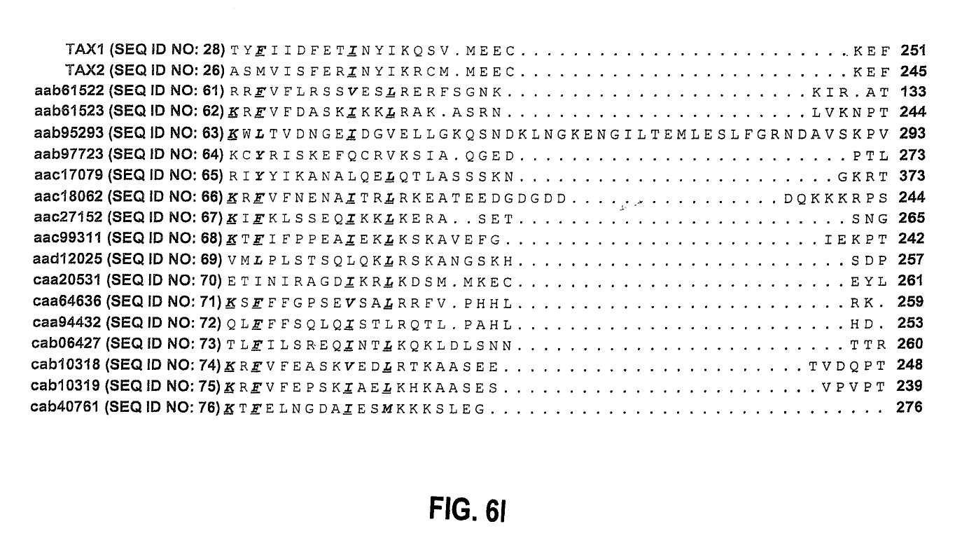

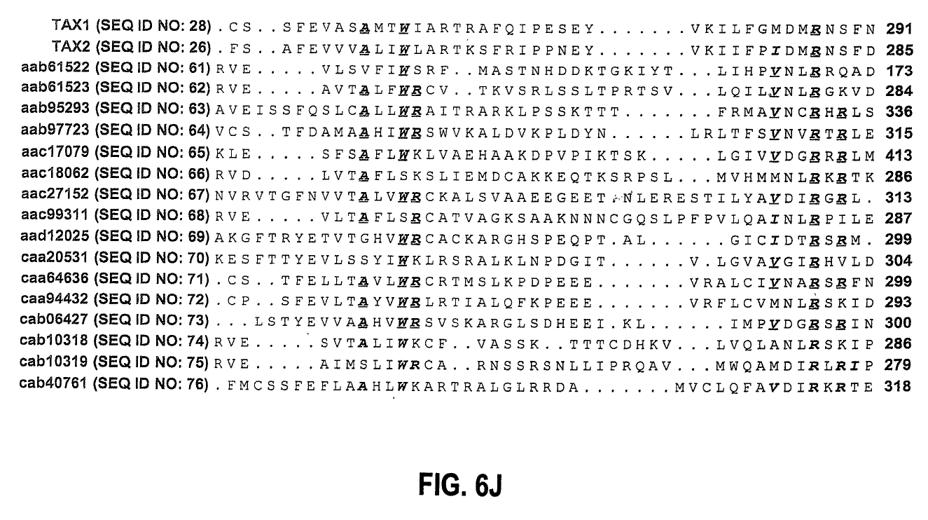

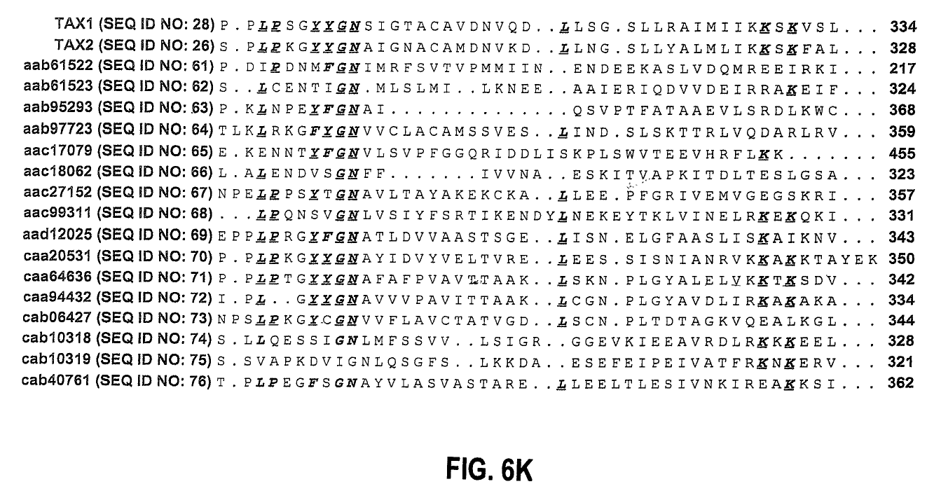

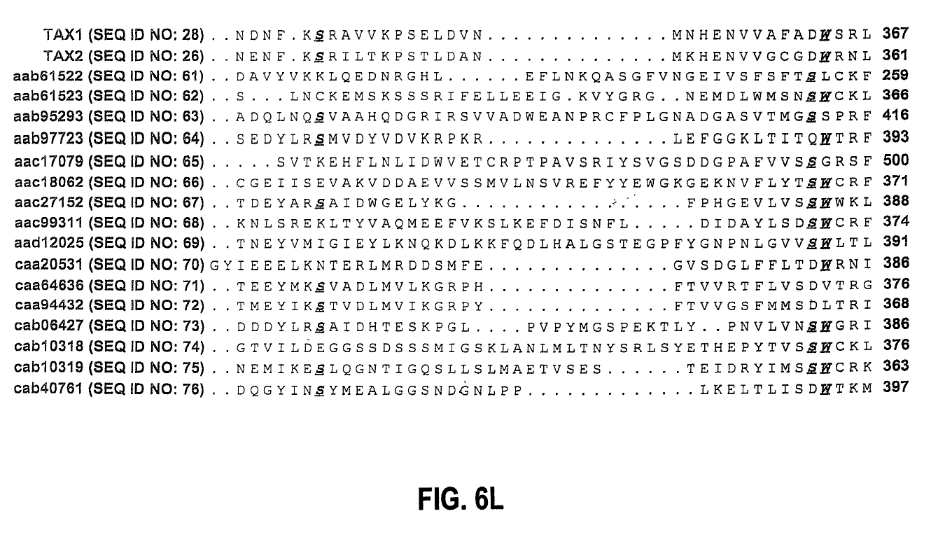

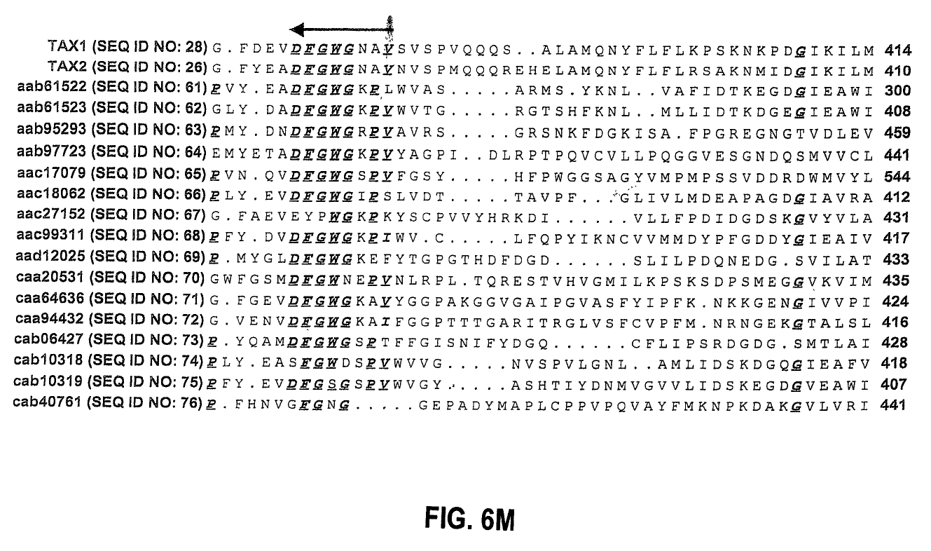

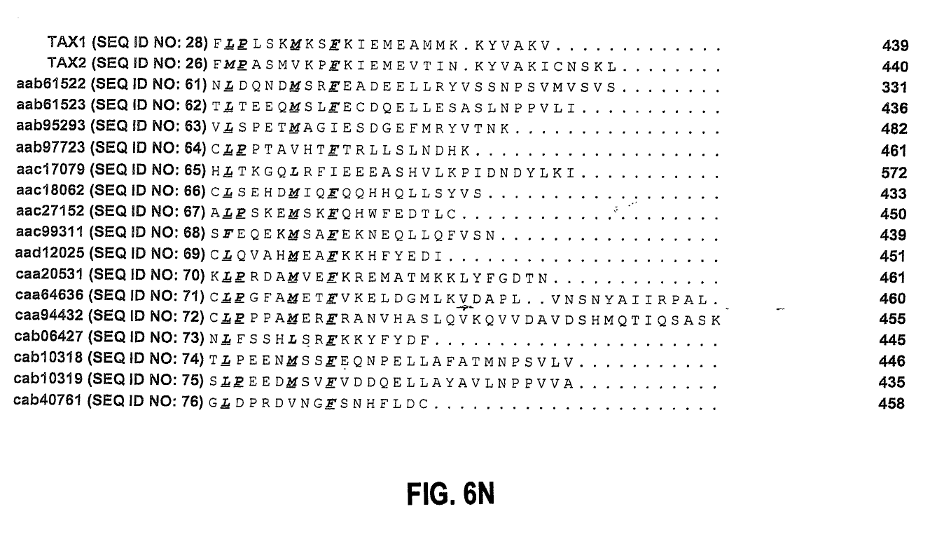

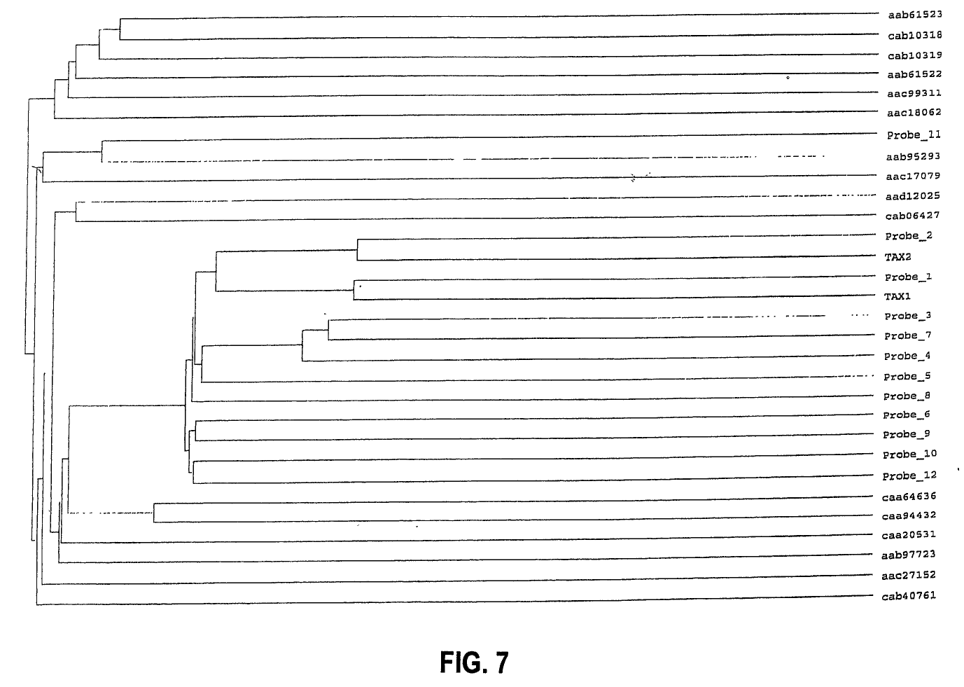

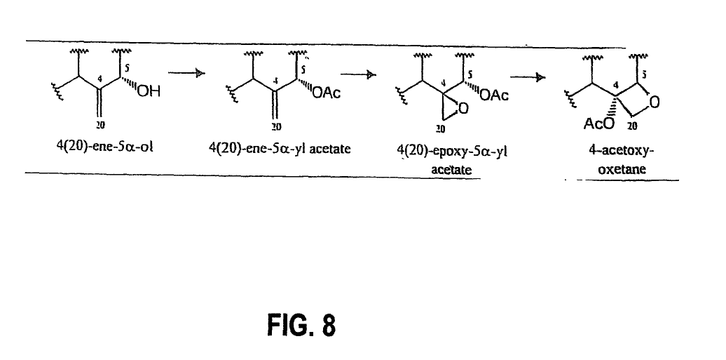

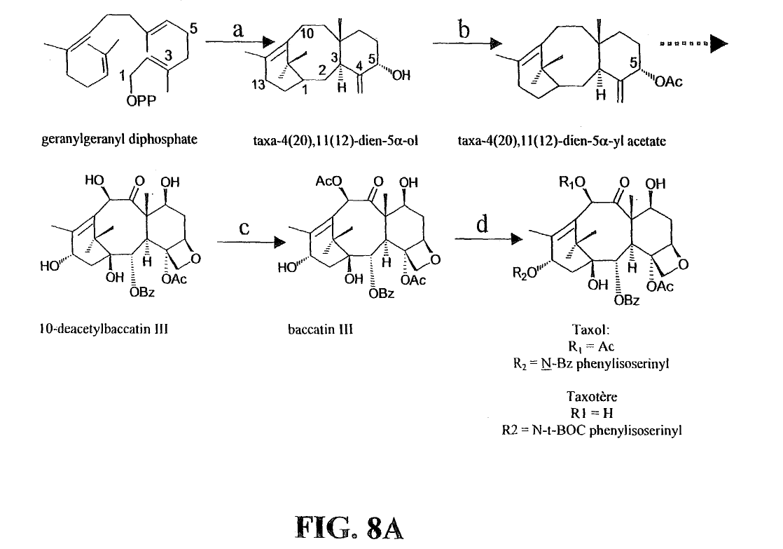

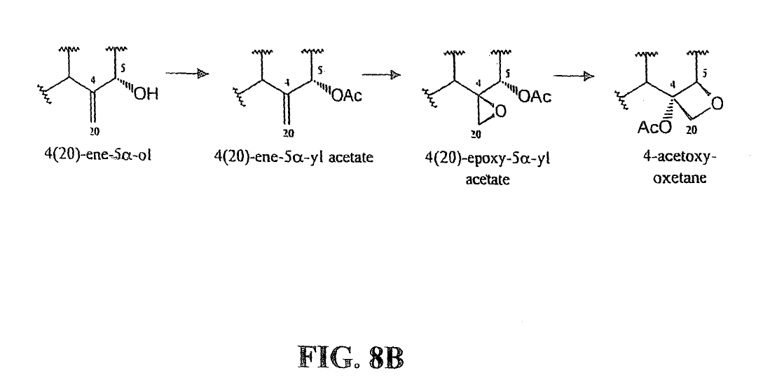

TABLE 2
Database (GenBank) sequences used for peptide comparisons.
For alignment, see FIG. 6; for placement in
dendrogram, see FIG. 7. The accession number is followed by
a two-letter code indicating genus and species
(AT, Arabidopsis thaliana; CM, Cucumis melo;
CR, Catharanthus roseus; DC, Dianthus caryophyllus; CB,
Clarkia breweri; NT, Nicotiana tabacum).

| | Protein | |
|---|---|---|
| Accession No. | Identification No. | Function |
| AC000103_AT | g2213627 | unknown; from genomic sequence |
| SEQ ID NO: 61 | | for Arabidopsis thaliana |
| | | BAC F21J9 |
| AC000103_AT | g2213628 | unknown; from genomic sequence |
| SEQ ID NO: 62 | | for A. thaliana BAC F21J9 |
| AF002109_AT | g2088651 | unknown; hypersensitivity-related |
| SEQ ID NO: 63 | | gene 201 isolog |
| AC002560_AT | g2809263 | unknown; from genomic sequence |
| SEQ ID NO: 64 | | for A. thaliana BAC F21B7 |
| AC002986_AT | g3152598 | unknown; similarity to C2-HC |
| SEQ ID NO: 65 | | type zinc finger protein C.e-MyT1 |
| | | gb/U67079 from C. elegans and |
| | | to hypersensitivity-related gene |
| | | 201 isolog T28M21.14 from |
| | | A. thaliana BAC |
| AC002392_AT | g3176709 | putative anthranilate |
| SEQ ID NO: 69 | | N-hydroxycinnamoyl/ |
| | | benzoyltransferase |
| AL031369_AT | g3482975 | unknown; putative protein |
| SEQ ID NO: 70 | | |
| Z84383_AT | g2239083 | hydroxycinnamoyl: benzoyl-CoA: |
| SEQ ID NO: 73 | | anthranilate N-hydroxycinnamoyl: |
| | | benzoyl transferase |
| Z97338_AT | g2244896 | unknown; similar to HSR201 |
| SEQ ID NO: 74 | | protein N. tabacum |
| Z97338_AT | g2244897 | unknown; hypothetical protein |
| SEQ ID NO: 75 | | |
| AL049607_AT | g4584530 | unknown; putative protein |
| SEQ ID NO: 76 | | |
| AF043464_CB | g3170250 | acetyl CoA: benzylalcohol |
| SEQ ID NO: 66 | | acetyl transferase |
| Z70521_CM | g1843440 | unknown; expressed during |
| SEQ ID NO: 72 | | ripening of melon |
| | | (Cucumis melo L.) fruits |
| AF053307_CR | g4091808 | deacetylvindoline 4-O-acetyl |
| SEQ ID NO: 68 | | transferase |
| AC004512_DC | g3335350 | unknown; similar to gb/Z84386 |
| SEQ ID NO: 67 | | anthranilate N-hydroxycinnamoyl/ |
| | | benzoyltransferase from |
| | | Dianthus caryophyllus |
| X95343_NT | g1171577 | unknown; hypersensitive reaction |
| SEQ ID NO: 71 | | in tobacco |

PCR amplifications were performed using each combination of forward and reverse primers, and induced Taxus cell library cDNA as a target. The amplifications produced, by cloning and sequencing, twelve related but distinct amplicons (each ca. 900 bp) having origins from the various primers (Table 3). These amplicons are designated “Probe 1” through “Probe 12,” and their nucleotide and deduced amino acid sequences are listed as SEQ ID NOs: 1-24, respectively.

TABLE 3
Primer combinations, amplicons and acquired genes. The parentheses
and brackets are used to designate the primer pair used and the
corresponding frequency at which that primer pair amplified the probe.

| | Amplicon | | | | |
|---|---|---|---|---|---|
| | Size | | | Acquired Nucleic acid | |
| Primer Pair | (bp) | Frequency | Designation | Designation | Function |
| AT-FOR1/AT-REV1 | 920 | 7/12 | Probe 1 | TAX1 (full-length) | taxadienol acetyl |
| (AT-FOR2/AT-REV1) | | (12/31) | | SEQ ID NO: 27; SEQ ID NO: 28 | transferase |
| (FIG. 4) | SEQ ID NO: 1; SEQ ID NO: 2 | | | TAX2 (full-length) | taxane-2-O- |
| | | | | SEQ ID NO: 25; SEQ ID NO: 26 | benzoyl |
| | | | | | transferase |
| AT-FOR1/AT-REV1 | 920 | 7/12 | Probe 2 | robe 2 was not used, but likely | — |
| (AT-FOR2/AT-Rev1) | | (2/31) | | could have acquired TAX2 | |
| | | | | because the sequence | |
| | | | | corresponds directly to this | |
| | | | | nucleic acid. | |
| (FIG. 4) | SEQ ID NO: 3; SEQ ID NO: 4 | | | | |
| AT-FOR4/AT-REV1 | 903 | 2/29 | Probe 3 | — | — |
| (FIG. 4) | SEQ ID NO: 5; SEQ ID NO: 6 | | | | |
| AT-FOR3/AT-REV1 | 908 | 1/29 | Probe 4 | — | — |
| (FIG. 4) | SEQ ID NO: 7; SEQ ID NO: 8 | | | — | — |
| AT-FOR4/AT-REV1 | 1297 | 1/32 | Probe 5 | TAX5 (full-length) | unknown |
| | | | | SEQ ID NO: 49; SEQ ID NO: 50 | |
| (FIG. 4) | SEQ ID NO: 9; SEQ ID NO: 10 | | | — | — |
| AT-FOR2/AT-REV1 | 911 | 8/32 | Probe 6 | TAX6 (full-length) | 10- |
| (AT-FOR3/AT-REV1) | | (1/29) | | SEQ ID NO: 44; Seq. ID No: 45 | deacetylbaccatin |
| [AT-FOR4/AT-REV1] | | [1/32] | | | III-10-O-acetyl |
| | | | | | transferase |
| (FIG. 4) | SEQ ID NO: 11; | | | — | — |
| | SEQ ID NO: 12 | | | | |
| AT-FOR3/AT-REV1 | 968 | 6/29 | Probe 7 | TAX7 (full-length) | c-13 |
| | | | | SEQ ID NO: 51; SEQ ID NO: 52 | phenylpropanoid |
| | | | | | side chain-CoA |
| | | | | | acyltransferase |
| (FIG.4) | SEQ ID NO: 13; | | | — | — |
| | SEQ ID NO: 14 | | | | |
| AT-FOR3/AT-REV1 | 908 | 1/29 | Probe 8 | — | — |
| (AT-FOR4/AT-REV1) | | (2/32) | | | |
| (FIG. 4) | SEQ ID NO: 15; | | | — | — |
| | SEQ ID NO: 16 | | | | |
| AT-FOR2/AT-REV1 | 908 | 1/32 | Probe 9 | AX9 (full-length) | unknown |
| (AT-FOR3/AT-REV1) | | (5/29) | | SEQ ID NO: 59; SEQ ID NO: 60 | |
| (FIG. 4) | SEQ ID NO: 17; | | | — | — |
| | SEQ ID NO: 18 | | | | |
| AT-FOR4/AT-REV1 | 911 | 2/32 | Probe 10 | TAX 10 (full-length) | benzoyl-CoA:3′- |
| | | | | SEQ ID NO: 53; SEQ ID NO: 54 | N-debenzoyl-2′- |
| | | | | | deoxytaxol |
| | | | | | N-benzoyl- |
| | | | | | transferase |
| (FIG. 4) | SEQ ID NO: 19; | | | — | — |
| | SEQ ID NO: 20 | | | | |
| AT-FOR4/AT-REV1 | 911 | 1/32 | Probe 11 | — | — |
| (FIG. 4) | SEQ ID NO: 21; | | | — | — |
| | SEQ ID NO: 22 | | | | |
| AT-FOR3/AT-REV1 | 908 | 3/29 | Probe 12 | TAX12 (full-length) | unknown |
| (AT-FOR4/AT-REV1) | | (1/32) | | SEQ ID NO: 55; SEQ ID NO: 56 | |

| | | | |
|---|---|---|---|
| (FIG. 4) | SEQ ID NO: 23; | — | — |
| | SEQ ID NO: 24 | | |
| | TAX13 does not appear to | TAX13 (full-length) | unknown |
| | directly correspond to | SEQ ID NO: 57; SEQ ID NO: 58 | |
| | any of the above listed | | |
| | Probes | | |

Notably, Probe 1, derived from the primers AT-FOR1 (SEQ ID NO: 34) and AT-REV1 (SEQ ID NO: 38), amplified a ˜900 bp DNA fragment encoding, with near identity, the proteolytic peptides corresponding to SEQ ID NOs: 31-33 of the purified protein. These results suggested that the amplicon Probe 1 represented the target nucleic acid for taxadienol acetyl transacylase. Probe 1 was then ³²P-labeled and employed as a hybridization probe in a screen of the methyl jasmonate-induced T. cuspidata suspension cell λZAP II™ cDNA library. Standard hybridization and purification procedures ultimately led to the isolation of three full-length, unique clones designated TAX1, TAX2, and TAX6 (SEQ ID NOS: 27,25, and 44, respectively). The clones and encoded proteins of TAX7 (SEQ ID NOS: 51-52) and TAX10 (SEQ ID NOS: 53-54) are discussed in Example 3 and 4 below.

Sequence Analysis and Functional Expression

Clone TAX1 (SEQ ID NO: 27) bears an open reading frame of 1317 nt and encodes a deduced protein (SEQ ID NO: 28) of 439 aa with a calculated molecular weight of 49,079 Da. Clone TAX2 (SEQ ID NO: 25) bears an open reading frame of 1320 nt and encodes a deduced protein (SEQ ID NO: 26) of 440 aa with a calculated molecular weight of 50,089 Da. Clone TAX6 (SEQ ID NO: 44) bears an open reading frame of 1320 nt and encodes a deduced protein (SEQ ID NO: 45) of 440 aa with a calculated molecular weight of 49,000 Da.

The sizes of TAX1 and TAX2 are consistent with the molecular weight of the native taxadienol transacetylase (MW ˜50,000) determined by gel-permeation chromatography (Walker et al., Arch. Biochem. Biophys. 364:273-279, 1999) and SDS polyacrylamide gel electrophoresis (SDS-PAGE). The deduced amino acid sequences of both TAX1 and TAX2 also remotely resemble those of other acetyl transacylases (50-56% identity; 64-67% similarity) involved in different pathways of secondary metabolism in plants (St. Pierre et al., Plant J. 14:703-713, 1998; and Dudareva et al., Plant J. 14:297-304, 1998). When compared to the amino acid sequence information from the tryptic peptide fragments, TAX1 exhibited a very close match (91% identity), whereas TAX2 exhibited conservative differences (70% identity).

The TAX6 calculated molecular weight of 49,052 Da is consistent with that of the native TAX6 protein (˜50 kDa), determined by gel permeation chromatography, indicating the protein to be a functional monomer, and is very similar to the size of the related, monomeric taxadien-5α-ol transacetylase (MW=49,079). The acetyl CoA: 10-deacetylbacctin III-10-O-acetyl transferase from Taxus cuspidata appears to be substantially different in size from the acetyl CoA: 10-hydroxytaxane-O-acetyl transferase recently isolated from Taxus chinensis and reported at a molecular weight of 71,000 (Menhard and Zenk, Phytochemistry 50:763-774, 1999).

The deduced amino acid sequence of TAX6 resembles that of TAX1 (64% identity; 80% similarity) and those of other acetyl transferases (56-57% identity; 65-67% similarity) involved in different pathways of secondary metabolism in plants (Dudareva et al., Plant J. 14:297-304, 1998; St-Pierre et al., Plant J. 14:703-713, 1998). Additionally, TAX6 possesses the HXXXDG (SEQ ID NO: 48) (residues H162, D166, and G167, respectively) motif found in other acyl transferases (Brown et al., J. Biol. Chem. 269:19157-19162, 1994; Carbini and Hersh, J. Neurochem. 61:247-253, 1993; Hendle et al., Biochemistry 34:4287-4298, 1995; and Lewendon et al., Biochemistry 33:1944-1950, 1994); this sequence element has been suggested to function in acyl group transfer from acyl CoA to the substrate alcohol (St. Pierre et al., Plant J. 14:703-713, 1998).

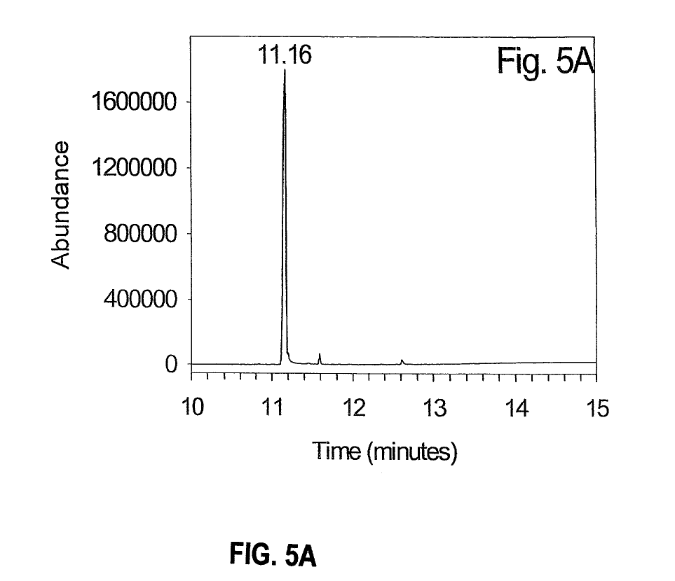

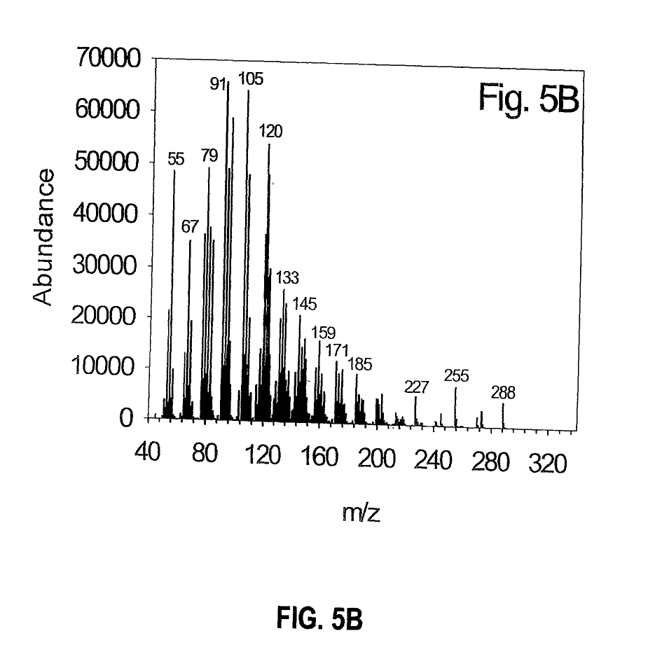

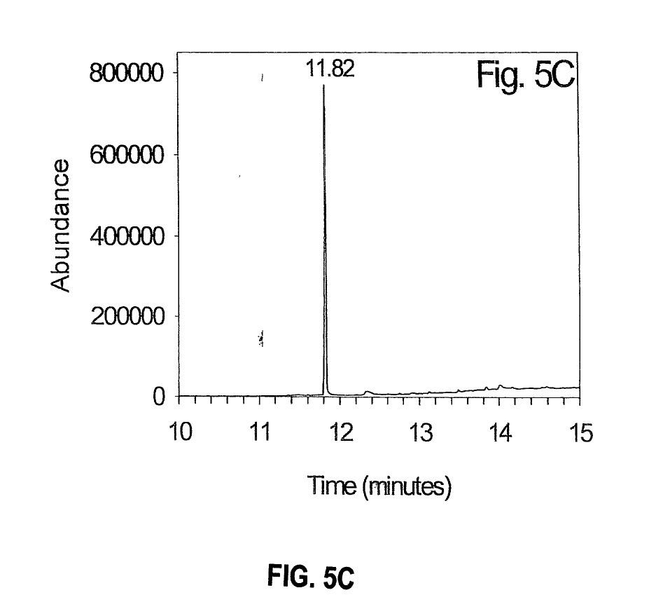

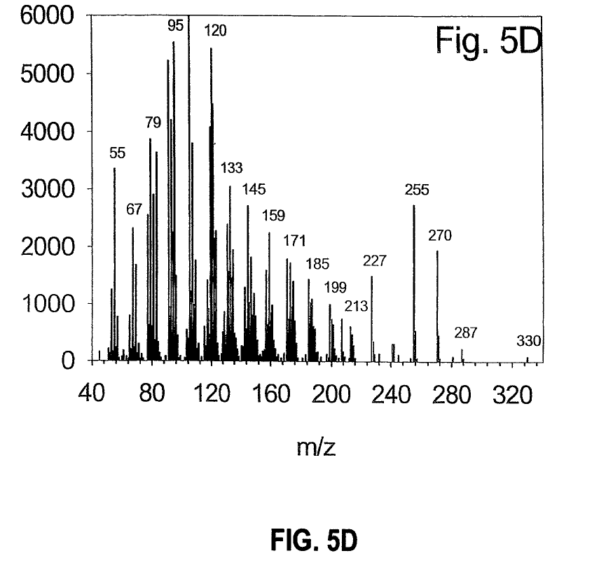

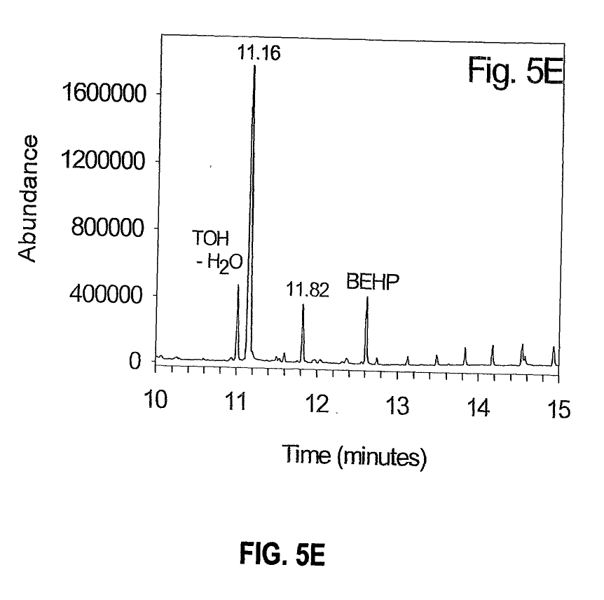

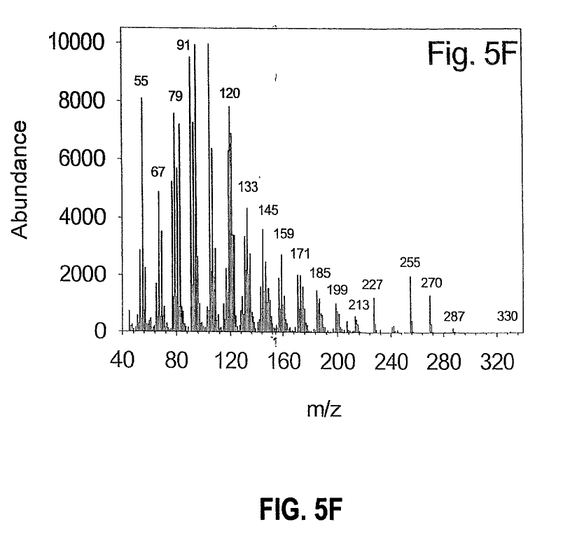

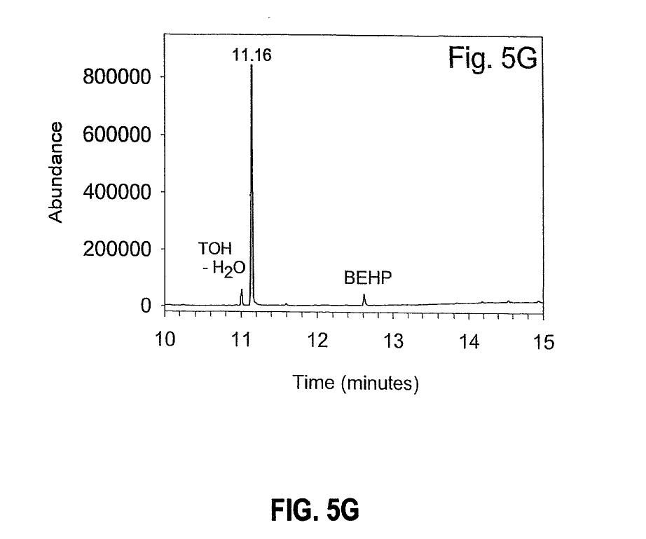

To determine the identity of the putative taxadienol acetyl transacylase, TAX1, TAX2, and TAX6 were subcloned in-frame into the expression vector pCWori+ (Barnes, Methods Enzymol. 272:3-14, 1996) and expressed in E. coli JM109 cells. The transformed bacteria were cultured and induced with isopropyl β-D-thiogalactoside (IPTG), and cell-free extracts were prepared and evaluated for taxadienol acetyl transacylase activity using the previously developed assay procedures (Walker et al., Arch. Biochem. Biophys. 364:273-279, 1999). Clone TAX1 (corresponding directly to Probe 1) expressed high levels of taxadienol acetyl transacylase activity (20% conversion of substrate to product), as determined by radiochemical analysis; the product of this recombinant enzyme was confirmed as taxadienyl-5α-yl acetate by gas chromatography-mass spectrometry (GC-MS) (FIGS. 5A-5G). Clone TAX2 did not express taxadienol acetyl transacylase activity and was inactive with the [³H]taxadienol and acetyl CoA co-substrates. TAX2 was later found to encode a taxane-2-O-benzoyl transferase. Neither of the recombinant proteins expressed from TAX1 or TAX2 was capable of acetylating the advanced Taxol® precursor 10-deacetyl baccatin III to baccatin III. Thus, based on the demonstration of functionally expressed activity, and the resemblance of the recombinant enzyme in substrate specificity and other physical and chemical properties to the native form, clone TAX1 was confirmed to encode the Taxus taxadienol acetyl transacylase.

Additionally, the heterologously expressed TAX6 was partially purified by anion-exchange chromatography (O-diethylaminoethylcellulose, Whatman, Clifton, N.J.) and ultrafiltration (Amicon Diaflo YM 10 membrane, Millipore, Bedford, Mass.) to remove interfering hydrolases from the bacterial extract, and the recombinant enzyme was determined to catalyze the conversion of 10-deacetylbaccatin III to baccatin III; the latter is the last diterpene intermediate in the Taxol® (paclitaxel) biosynthetic pathway. The optimum pH for TAX6 was determined to be 7.5, with half-maximal velocities at pH 6.4 and 7.8. The Kₘ values for 10-deacetylbaccatin III and acetyl CoA were determined to be 10 μM and 8 μM, respectively, by Lineweaver-Burk analysis (for both plots R²=0.97). These kinetic constants for TAX6 are comparable to the taxa-4(20),11(12)-dien-5α-ol acetyl transferase possessing Kₘ values for taxadienol and acetyl CoA of 4 μM and 6 μM, respectively. The TAX6 enzyme appears to acetylate the 10-hydroxyl group of taxoids with a high degree of regioselectivity, since the enzyme does not acetylate the 1β-, 7β-, or 13α-hydroxyl groups of 10-deacetylbaccatin III, nor does it acetylate the 5α-hydroxyl group of taxa-4(20),11(12)-dien-5α-ol.

### Example 2

### Isolating a Nucleic Acid Encoding acetyl CoA:taxa-4(20),11(12)-dien-5α-ol O-acetyl transacylase

Overview

A newly designed isolation and purification method is described herein for the preparation of homogeneous taxadien-5α-ol acetyl transacylase from Taxus canadensis. The purified protein was N-terminally blocked, thereby requiring internal amino acid microsequencing of fragments generated by proteolytic digestion. Peptide fragments so generated were purified by HPLC and sequenced, and one suitable sequence was used to design a set of degenerate PCR primers. Several primer combinations were employed to amplify a series of twelve related, DNA sequences (Probes 1-12; see Table 3). Nine of these DNA sequences were used as hybridization probes to screen an induced Taxus cuspidata cell cDNA library. This strategy allowed for the successful isolation of eight full-length transacylase cDNA clones. The identity of one of these clones was confirmed by sequence matching to the peptide fragments described herein and by heterologous functional expression of transacylase activity in Escherichia coli.

Culture of Cells

Initiation, propagation and induction of Taxus sp. cell cultures, reagents, procedures for the synthesis of substrates and standards, and general methods for transacylase isolation, characterization and assay have been previously described (Hefner et al., Arch. Biochem. Biophys. 360:62-75, 1998; and Walker et al., Arch. Biochem. Biophys. 364:273-279, 1999). Since all designated Taxus species are considered to be closely related subspecies (Bolsinger and Jaramillo, Silvics of Forest Trees of North America (revised), Pacific Northwest Research Station, USDA, Portland, Oreg., 1990; and Voliotis, Isr. J. Botany 35:47-52, 1986), the Taxus cell sources were chosen for operational considerations because only minor sequence differences and/or allelic variants between proteins and genes of the various “species” were expected. Thus, Taxus canadensis cells were chosen as the source of transacetylase, because they express transacetylase at high levels, and Taxus cuspidata cells were selected for cDNA library construction because they produce Taxol® at high levels.

Isolation and Purification of the Enzyme

No related terpenol transacylase genes are available in the databases (see below) to permit homology-based cloning. Hence, a protein-based (reverse genetic) approach to cloning the target transacetylase was utilized. This reverse genetic approach required obtaining a partial amino acid sequence, generating degenerate primers, amplifying a portion of cDNA using PCR, and using the amplified fragment as a probe to detect the correct clone in a cDNA library.

Unfortunately, the previously described partial protein purification protocol, including an affinity chromatography step, did not yield pure protein for amino acid microsequencing, nor did the protocol yield protein in useful amounts, or provide a sufficiently simplified SDS-PAGE banding pattern to allow assignment of the transacetylase activity to a specific protein (Walker et al., Arch. Biochem. Biophys. 364:273-279, 1999). Furthermore, numerous variations on the affinity chromatography step, as well as the earlier anion exchange and hydrophobic interaction chromatography steps, failed to improve the specific activity of the preparations due to the instability of the enzyme upon manipulation. Also, a five-fold increase in the scale of the preparation resulted in only marginally improved recovery (generally<5% total yield accompanied by removal of>99% of total starting protein). Furthermore, because the enzyme could not be purified to homogeneity, and attempts to improve stability by the addition of polyols (sucrose, glycerol), reducing agents (Na₂S₂O₅, ascorbate, dithiothreitol, β-mercaptoethanol), and other proteins (albumin, casein) also were not productive (Walker et al., Arch. Biochem. Biophys. 364:273-279, 1999), this approach was abandoned.

To overcome the problem described above, the following isolation and purification procedure was used. The purity of the taxadienol acetyl transacylase after each fractionation step was assessed by SDS-PAGE according to Laemmli (Laemmli, Nature 227:680-685, 1970); quantification of total protein after each purification step was carried out by the method of Bradford, Analytical Biochem. 72:248-254, 1976, or by Coommassie Blue staining, and transacylase activity was assessed using the methods described in Walker et al., Arch. Biochem. Biophys. 364:273-279, 1999.

Procedures for protein staining have been described (Wray et al., Anal. Biochem. 118:197-203, 1991). The preparation of the T. canadensis cell-free extracts and all subsequent procedures were performed at 0-4° C. unless otherwise noted. Cells (40 g batches) were frozen in liquid nitrogen and thoroughly pulverized for 1.5 minutes using a mortar and pestle. The resulting frozen powder was transferred to 225 mL of ice cold 30 mM HEPES buffer (pH 7.4) containing 3 mM dithiothreitol (DTT), XAD-4 polystyrene resin (12 g) and polyvinylpolypyrrolidone (PVPP, 12 g) to adsorb low molecular weight resinous and phenolic compounds. The slurry was slowly stirred for 30 minutes, and the mixture was filtered through four layers of cheese cloth to remove solid absorbents and particulates. The filtrate was centrifuged at 7000 g for 30 minutes to remove cellular debris, then at 100,000 g for 3 hours, followed by 0.2-μm filtration to yield a soluble protein fraction (in ˜200 mL buffer) used as the enzyme source.

The soluble enzyme fraction was subjected to ultrafiltration (DIAFLO™ YM 30 membrane, Millipore, Bedford, Mass.) to concentrate the fraction from 200 mL to 40 mL and to selectively remove proteins of molecular weight lower than the taxadien-5α-ol acetyl transacylase (previously established at 50,000 Da in Walker et al., Arch. Biochem. Biophys. 364:273-279, 1999). Using a peristaltic pump, the concentrate (40 mL) was applied (2 mL/minute) to a column of O-diethylaminoethylcellulose (2.8×10 cm, Whatman DE-52, Fairfield, N.J.) that had been equilibrated with “equilibration buffer” (30 mM HEPES buffer (pH 7.4) containing 3 mM DTT). After washing with 60 mL of equilibration buffer to remove unbound material, the proteins were eluted with a step gradient of the same buffer containing 50 mM (25 mL), 125 mM (50 mL), and 200 mM (50 mL) NaCl.

The fractions were assayed as described previously (Walker et al., Arch. Biochem. Biophys. 364:273-279, 1999), and those containing taxadien-5α-ol acetyl transacylase activity (125-mM and 200-mM fractions) were combined (100 mL, ˜160 mM) and diluted to 5 mM NaCl (160 mL) by ultrafiltration (DIAFLO™ YM 30 membrane, Millipore, Bedford, Mass.) and repeated dilution with 30 mM HEPES buffer (pH 7.4) containing 3 mM DTT.

Further purification was effected by high-resolution anion-exchange and hydroxyapatite chromatography run on a Pharmacia FPLC system coupled to a 280-nm effluent detector. The preparation described above was applied to a preparative anion-exchange column (10×100 mm, Source 15Q, Pharmacia Biotech., Piscataway, N.J.) that was previously washed with “wash buffer” (30 mM HEPES buffer (pH 7.4) containing 3 mM DTT) and 1 M NaCl, and then equilibrated with wash buffer (without NaCl). After removing unbound material, the applied protein was eluted with a linear gradient of 0 to 200 mM NaCl in equilibration buffer (215 mL total volume; 3 mL/minute) (see FIG. 3A). Fractions containing transacetylase activity (eluting at ˜80 mM NaCl) were combined and diluted to 5 mM NaCl by ultrafiltration using 30 mM HEPES buffer (pH 7.4) containing 3 mM DTT as diluent, as described above. The desalted protein sample (70 mL) was loaded onto an analytical anion-exchange column (5×50 mm, Source 15Q, Pharmacia Biotech., Piscataway, N.J.) that was washed and equilibrated as before. The column was developed using a shallow, linear salt gradient with elution to 200 mM NaCl (275 mL total volume, 1.5 mL/minute, 3.0 mL fractions). The taxadienol acetyl transacylase eluted at ˜55-60 mM NaCl (see FIG. 3B), and the appropriate fractions were combined (15 mL), reconstituted to 45 mL in 30 mM HEPES buffer (pH 6.9) and applied to a ceramic hydroxyapatite column (10×100 mm, Bio-Rad Laboratories, Hercules, Calif.) that was previously washed with 200 mM sodium phosphate buffer (pH 6.9) and then equilibrated with an “equilibration buffer” (30 mM HEPES buffer (pH 6.9) containing 3 mM DTT (without sodium phosphate)). The equilibration buffer was used to desorb weakly associated material, and the bound protein was eluted by a gradient from 0 to 40 mM sodium phosphate in equilibration buffer (125 mL total volume, at 3.0 mL/minute, 3.0 mL fractions) (see FIG. 3C). The fractions containing the highest activity, eluting over 27 mL at 10 mM sodium phosphate, were combined and shown by SDS-PAGE to yield a protein of ˜95% purity (a minor contaminant was present at ˜35 kDa, see FIG. 3D). The level of transacylase activity was measured after each step in the isolation and purification protocol described above. The level of activity recovered is shown in Table 4.

TABLE 4
Summary of taxadien-5α-ol O-acetyl transferase
purification from Taxus cells.

| | | | Specific | |
|---|---|---|---|---|
| | Total | Total | Activity | |
| | activity | Protein | (pkat/mg | Purification |
| | (pkat) | (mg) | protein) | (fold) |
| Crude extract | 302 | 1230 | 0.25 | 1 |
| YM30 ultrafiltration | 136 | 98 | 1.4 | 5.6 |
| DE-52 | 122 | 69 | 1.8 | 7.2 |
| YM30 ultrafiltration | 54 | 55 | 1.0 | 4 |
| Source 15Q | 47 | 3 | 16 | 63 |
| (10 × 100 mm) | | | | |
| YM3O ultrafiltration | 19 | 2.6 | 7.3 | 29 |
| Source 15Q | 13 | 0.12 | 108 | 400 |
| (5 × 50 mm) | | | | |
| Hydroxyapatite | 10 | 0.05 | 200 | 800 |

Amino Acid Microsequencing of Taxadienol Acetyl Transacylase

The purified protein from multiple preparations as described above (>95% pure, ˜100 pmol, 50 μg) was subjected to preparative SDS-PAGE (Laemmli, Nature 227:680-685, 1970). The protein band at 50 kDa, corresponding to the taxadienol acetyl transacylase, was excised. Whereas treatment with V8 protease or treatment with cyanogen bromide (CNBr) failed to yield sequencable peptides, in situ proteolysis with endolysC (Caltech Sequence/Structure Analysis Facility, Pasadena, Calif.) and trypsin (Fernandez et al., Anal Biochem. 218:112-118, 1994) yielded a number of peptides, as determined by HPLC, and several of these were separated, verified by mass spectrometry (Fernandez et al., Electrophoresis 19:1036-1045, 1998), and subjected to Edman degradative sequencing, from which five distinct and unique amino acid sequences (designated SEQ ID NOs: 29-33) were obtained (FIG. 2).

cDNA Library Construction and Related Manipulations

A cDNA library was constructed from mRNA isolated from T. cuspidata suspension culture cells that had been induced to maximal Taxol® production with methyl jasmonate for 16 hours. An optimized protocol for the isolation of total RNA from T. cuspidata cells was developed empirically using a buffer containing 100 mM Tri-HCl (pH 7.5), 4 M guanidine thiocyanate, 25 mM EDTA and 14 mM β-mercaptoethanol. Cells (1.5 g) were disrupted at 0-4° C. using a Polytron™ ultrasonicator (Kinematica AG, Switzerland; 4×15 second bursts at power setting 7), the resulting homogenate was adjusted to 2% (v/v) Triton X-100 and allowed to stand 15 minutes on ice. An equal volume of 3 M sodium acetate (pH 6.0) was then added, and the mixed solution was incubated on ice for an additional 15 minutes, followed by centrifugation at 15,000g for 30 minutes at 4° C. The resulting supernatant was mixed with 0.8 volume of isopropanol and allowed to stand on ice for 5 minutes, followed by centrifugation at 15,000 g for 30 minutes at 4° C. The resulting pellet was dissolved in 8 mL of 20 mM Tris-HCl (pH 8.0) containing 1 mM EDTA, adjusted to pH 7.0 by addition of 2 mL of 2 M NaCl in 250 mM MOPS buffer (pH 7.0), and total RNA was recovered by passing this solution over a nucleic acid isolation column (Qiagen, Inc.; Valencia, Calif.) following the manufacturer's instructions. Poly(A)+ mRNA was then purified from total RNA by chromatography on oligo(dT) beads (Oligotex™ mRNA Kit, Qiagen, Inc.), and this material was used to construct a library using the λZAPII™ cDNA synthesis kit and Gigapack™ III gold packaging kit from Stratagene Corp. (La Jolla, Calif.) by following the manufacturer's instructions.

Unless otherwise stated, standard methods were used for DNA manipulations and cloning (see, e.g., Innis et al., 1999, and Sambrook et al., 2001). DNA was sequenced using Amplitaq™ (Hoffmann-La Roche Inc., Nutley, N.J.) DNA polymerase and cycle sequencing (fluorescence sequencing) on an ABI Prism™ 373 DNA Sequencer. The E. coli strains XL1-Blue and XL1-Blue MRF′ (Stratagene Corp.; La Jolla, Calif.) were used for routine cloning of PCR products and for cDNA library construction, respectively. E. coli XL1-Blue MRF′cells were used for in vivo excision of purified pBluescript SK from positive plaques and the excised plasmids were used to transform E. coli SOLR cells.

Degenerate Primer Design and PCR Amplification

Due to codon degeneracy, only one sequence of the five tryptic peptide fragments obtained (SEQ ID NO: 30 of FIG. 2) was suitable for PCR primer construction. Two such degenerate forward primers, designated AT-FOR1 (SEQ ID NO: 34) and AT-FOR2 (SEQ ID NO: 35), were designed based on this sequence (FIG. 4). Using the NCBI Blast 2.0 database searching program (Genetics computer Group, Program Manual for the Wisconsin Package, version 9, Genetics computer Group, 575 Science Drive, Madison, Wisc., 1994) to search for this sequence element among the few defined transacylases of plant origin (St. Pierre et al., Plant J. 14:703-713, 1998; Dudareva et al., Plant J. 14:297-304, 1998; and Yang et al., Plant Mol. Bio. 35:777-789, 1997), and the many deposited sequences of unknown function, allowed the identification of two possible sequence variants of this element (FYPFAGR (SEQ ID NO: 39) and YYPLAGR (SEQ ID NO: 40)) from which two additional degenerate forward primers, designated AT-FOR3 (SEQ ID NO: 36) and AT-FOR4 (SEQ ID NO: 37), were designed (FIG. 4). The sequences employed for this comparison are listed in Table 1. Using this range of functionally defined and undefined sequences, conserved regions were sought for the purpose of designing a degenerate reverse primer (the distinct lack of similarity of the Taxus sequences to genes in the database can be appreciated by reference to FIGS. 6A-6N), from which one such consensus sequence element (DFGWGKP) (SEQ ID NO: 41) was noted, and was employed for the design of the reverse primer AT-REV1 (SEQ ID NO: 38) (FIG. 4). This set of four forward primers and one reverse primer incorporated a varied number of inosines, and ranged from 72- to 216-fold degeneracy. The remaining four proteolytic peptide fragment sequences (SEQ ID NO: 29, SEQ ID NO: 31, SEQ ID NO: 32, SEQ ID NO: 33 of FIG. 2) were less suitable for primer design, and were not found (by NCBI BLAST™ searching) to be similar to other related sequences, thus suggesting that these represented more specific sequence elements of the Taxus transacetylase nucleic acid.

Each forward primer (150 μM) and the reverse primer (150 μM) were used in separate PCR reactions performed with Taq polymerase (3 U/100 μL reaction containing 2 mM MgCl₂) and employing the induced T. cuspidata cell cDNA library (10⁸ PFU) as template under the following conditions: 94° C. for 5 minutes, 32 cycles at 94° C. for 1 minute, 40° C. for 1 minute and 74° C. for 2 minutes and, finally, 74° C. for 5 minutes. The resulting amplicons (regions amplified by the various primer combinations) were analyzed by agarose gel electrophoresis and the products were extracted from the gel, ligated into pCR TOPOT7 (Invitrogen Corp.; Carlsbad, Calif.), and transformed into E. Coli TOPI0F′ cells (invitrogen, Carlsbad, Calif.). Plasmid DNA was prepared from individual transformants and the inserts were fully sequenced.

The combination of primers AT-FOR1 (SEQ ID NO: 34) and AT-REV1 (SEQ ID NO: 38) yielded a 900-bp amplicon. Cloning and sequencing of the amplicon revealed two unique sequences designated “Probe 1” (SEQ ID NO: 1) and “Probe 2” (SEQ ID NO: 3) (Table 3). The results with the remaining primer combinations are provided in Table 3.

Library screening

Four separate library-screening experiments were designed using various combinations of the radio-labeled amplicons (Probes 1-12) as probes. Use of radio-labeled Probe 1 (SEQ ID NO: 1), led to the identification of TAX1 (SEQ ID NO: 27) and TAX2 (SEQ ID NO: 25), and use of radio-labeled Probe 6 (SEQ ID NO: 11) led to the identification of TAX6 (SEQ ID NO: 44). A probe consisting of a mixture of radio-labeled Probe 10 (SEQ ID NO: 19) and Probe 12 (SEQ ID NO: 23) led to the identification of TAX10 (SEQ ID NO: 44) and TAX12 (SEQ ID NO: 55). Finally, a probe containing a mixture of radio-labeled Probes 3, 4, 5, 7, and 9 led to the identification of TAX5, TAX 7, and TAX13 (SEQ ID NOs. 49, 51, and 57, respectively). Details of these individual library-screening experiments are provided below.

The identification of TAX1 (SEQ ID NO: 27) and TAX2 (SEQ ID NO: 25) was accomplished using 1 μg of Probe 1 (SEQ ID NO: 1) that had been amplified by PCR, the resulting amplicon was gel-purified, randomly labeled with [α-³²P]CTP (Feinberg and Vogelstein, Anal. Biochem. 137:216-217, 1984), and used as a hybridization probe to screen membrane lifts of 5×10⁵ plaques grown in E. coli XL1-Blue MRF′. Phage DNA was cross-linked to the nylon membranes by autoclaving on fast cycle 3-4 minutes at 120° C. After cooling, the membranes were washed 5 minutes in 2×SSC, then 5 minutes in 6×SSC (containing 0.5% SDS, 5×Denhardt's reagent, 0.5 g Ficoll (Type 400, Pharmacia, Piscataway, N.J.), 0.5 g polyvinylpyrrolidone (PVP-10), and 0.5 g bovine serum albumin (Fraction V, Sigma, Saint Louis, Mo.) in 100 mL total volume). Hybridization was then performed for 20 hours at 68° C. in 6×SSC, 0.5% SDS and 5×Denhardt's reagent. The nylon membranes were then washed two times for 5 minutes in 2×SSC with 0.1% SDS at 25° C., and then washed 2×30 minutes with 1×SSC and 0.1% SDS at 68° C. After washing, the membranes were exposed for 17 hours to Kodak (Rochester, N.Y.) XAR film at −70° C.

Of the plaques exhibiting positive signals (˜600 total), 60 were purified through two additional rounds of hybridization. Purified λZAPII clones were excised in vivo as pBluescript II SK(−) phagemids and transformed into E. coli SOLR cells (Stratagene Corp.; La Jolla, Calif.). The size of each cDNA insert was determined by PCR using T3 and T7 promoter primers, and size-selected inserts (>1.5 kb) were partially sequenced from both ends to sort into unique sequence types and to acquire full-length versions of each (by further screening with a newly designed 5′-probe, if necessary).

The same basic screening protocol, as illustrated by the results provided below, can be repeated with all of the probes described in Table 3, with the goal of acquiring the full range of full-length, in-frame putative transacylase clones for test of function by expression in E. coli. In the case of Probe 1 (SEQ ID NO: 1), two unique full-length clones, designated TAX1 (SEQ ID NO: 27 and SEQ ID NO: 28) and TAX2 (SEQ ID NO: 25 and SEQ ID NO: 26), were isolated.

An additional transacylase, TAX6 (SEQ ID NO: 44), was identified by using 40 ng of radio-labeled Probe 6 (SEQ ID NO: 11) to screen the T. cuspidata library. This full-length clone was 99% identical to Probe 6 (SEQ ID NO: 11) and 99% identical to the deduced amino acid sequence of Probe 6 (SEQ ID NO: 12), indicating that the probe had located its cognate.

Using 40 ng of radio-labeled Probe 10 (SEQ ID NO: 19) and 40 ng of radio-labeled Probe 12 (SEQ ID NO: 23) led to the identification of the full-length transacylases TAX10 (SEQ ID NO: 53 and SEQ ID NO: 54) and TAX12 (SEQ ID NO: 55 and SEQ ID NO: 56) in separate hybridization screening experiments.

Use of a probe mixture containing about 6 ng each of Probes 3, 4, 5, 7, and 8 (SEQ ID NOs. 5, 7, 9, 13, and 15, respectively) randomly labeled with [α-³²P]CTP (Feinberg and Vogelstein, Ana. Biochem. 137:216-2 17, 1984) resulted in the identification of full-length transacylases TAX5 (SEQ ID NO: 49) and TAX7 (SEQ ID NO: 51), which correspond to Probes 5 (SEQ ID NO: 9) and 7 (SEQ ID NO: 13), respectively. An additional full-length transacylase, TAX13 (SEQ ID NO: 57) was also identified, however, this transacylase does not correspond to any of the Probes identified in Table 3.

cDNA Expression in E. coli

Full-length insert fragments of the relevant plasmids are excised and subcloned in-frame into the expression vector pCWori+ (Barnes, Methods Enzymol. 272:3-14, 1996). This procedure can involve the elimination of internal restriction sites and the addition of appropriate 5′- and 3′-restriction sites for directional ligation into the expression vector using standard PCR protocols (Innis et al., 1999) or commercial kits such as the Quick Change Mutagenesis System (Stratagene Corp.; La Jolla, Calif.). For example, the full-length transacylase corresponding to probe 6 (SEQ ID NO: 11) was obtained using the primer set (5′-GGGAATTCCATATGGCAGGCTCAACAGAATTTGTGG-3′ (SEQ ID NO: 46) and 3′-GTTTATACATTGATTCGGAACTAGATCTGATC-5′ (SEQ ID NO: 47)) to amplify the putative full-length acetyl transferase nucleic acid and incorporate NdeI and XbaI restriction sites at the 5′- and 3′-termini, respectively, for directional ligation into vector pCWori+ (Barnes, Methods Enzymol. 272:3-14, 1996). All recombinant pCWori+ plasmids are confirmed by sequencing to insure that no errors have been introduced by the polymerase reactions, and are then transformed into E. coli JM109 by standard methods.

Isolated transformants for each full-length insert are grown to A₆₀₀=0.5 at 37° C. in 50 mL Luria-Bertani medium supplemented with 50 μg ampicillin/mL, and a 1-mL inoculum added to a large scale (100 mL) culture of Terrific Broth (6 g bacto-tryptone, DIFCO Laboratories, Spark, Md., 12 g yeast extract, EM Science, Cherryhill, N.J., and 2 mL glycerol in 500 mL water) containing 50 μg ampicillin/mL and thiamine HCl (320 mM) and grown at 28° C. for 24 hours. Approximately 24 hours after induction with 1 mM isopropyl β-D-thiogalactoside (IPTG), the bacterial cells are harvested by centrifugation, disrupted by sonication in assay buffer consisting of 30 mM potassium phosphate (pH 7.4), or 25 mM MOPSO (pH 7.4), followed by centrifugation to yield a soluble enzyme preparation that can be assayed for transacylase activity.

Enzyme Assay

A specific assay for acetyl CoA:taxa-4(20),11(12)-dien-5α-ol O-acetyl transacylase has been described previously (Walker et al., Arch. Biochem. Biophys. 364:273-279, 1999, herein incorporated by reference). Generally the assay for taxoid acyltransacylases involves the CoA-dependent acyl transfer from acetyl CoA (or other acyl or aroyl CoA ester) to a taxane alcohol, and the isolation and chromatographic separation of the product ester for confirmation of structure by GC-MS (or HPLC-MS) analysis. For another example of such an assay, see Menhard and Zenk, Phytochemistry 50:763-774, 1999.

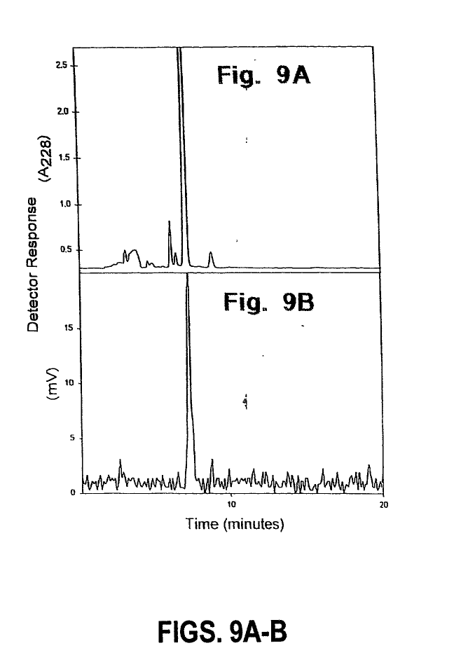

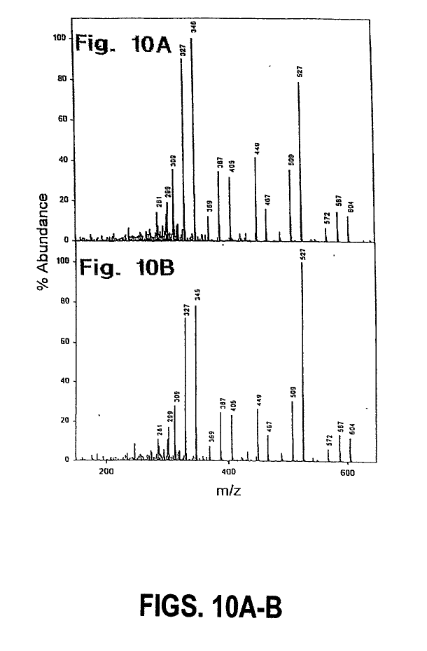

The activity of TAX6 (SEQ ID NO: 45) was assayed under standard conditions described in Walker et al., Arch. Biochem. Biophys. 364:273-279, 1999, with 10-deacetylbaccatin III (400 μM, Hauser Chemical Research Inc., Boulder, Colo.) and [2-³H]acetyl CoA (0.45 μCi, 400 μM (NEN, Boston, Mass.)) as co-substrates. The TAX6 (SEQ ID NO: 45) enzyme preparation yielded a single product from reversed-phase radio-HPLC analysis, with a retention time of 7.0 minutes (coincident radio and UV traces) corresponding exactly to that of authentic baccatin III (generously provided by Dr. David Bailey of Hauser Chemical Research Inc., Boulder, Colo.) (FIGS. 9A-9B). The identity of the biosynthetic product was further verified as baccatin III by combined LC-MS (liquid chromatography-mass spectrometry) analysis (FIGS. 10A-10B), which demonstrated the identical retention time (8.6×0.1 minute) and mass spectrum for the product and authentic standard. Finally, a sample of the biosynthetic product, purified by silica gel analytical TLC, gave a ¹H-NMR spectrum identical to that of authentic baccatin III, confirming the enzyme as 10-deacetylbaccatin III-10-O-acetyl transferase (TAX6 (SEQ ID NO: 45)) and also confirming that the corresponding nucleic acid had been isolated.

### Example 3

### Characterization of a C-13 phenylpropanoid Side Chain-CoA acyltransferase

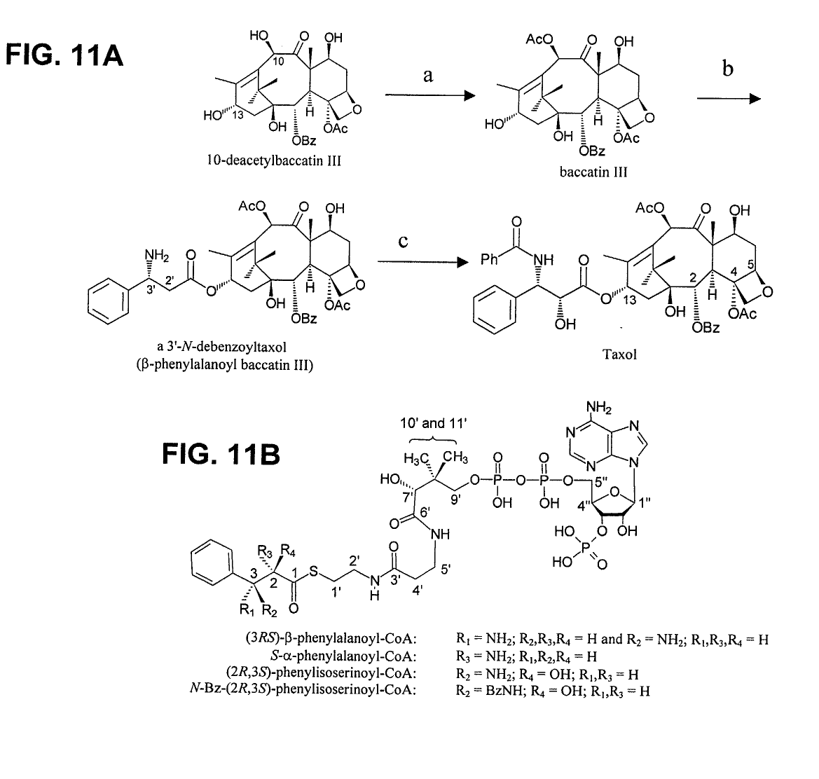

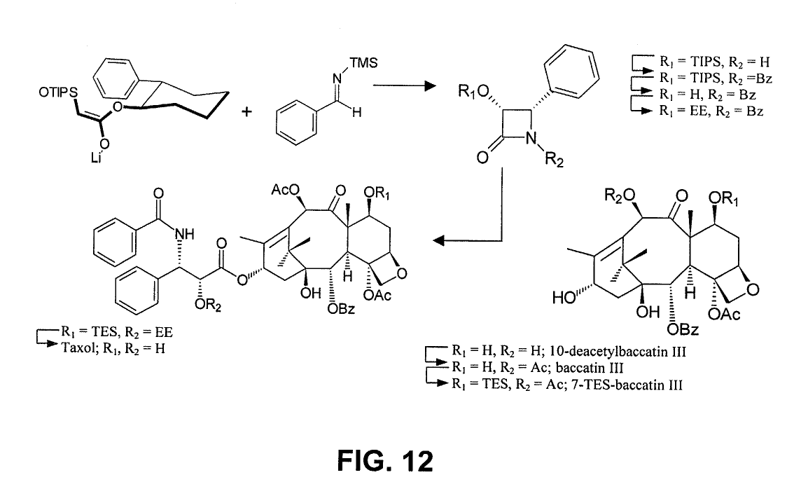

The Taxol® pharmacophore defined in chemical terms comprises, in part, a 13-O-(3-benzamido-3-phenylisoseryl) side chain, which is known to bind the N-terminus of the β-subunit of tubulin in Taxol® binding assays. A cDNA clone (designated TAX7 SEQ ID NO: 51) encoding a Taxol® C-13 O-phenylpropanoyltransferase was identified from the set of transacylases obtained from the cDNA library described in Examples 1 and 2. The recombinant enzyme (SEQ ID NO: 52) expressed in Escherichia coli catalyzes the selective 13-O-acylation of baccatin III with β-phenylalanoyl coenzyme A as the acyl donor to form N-debenzoyl-2′-deoxytaxol. The product was converted to 2′-deoxytaxol by chemical N-benzoylation, and the derivative was confirmed by various spectrometric analyses. The full-length cDNA has an open reading frame of 1,335 bases and encodes a 445 aa polypeptide with a calculated molecular weight of 50,546Da. Evaluation of the kinetic parameters for this transferase revealed Kₘ values of 2.6 μM and 4.5 μM for baccatin III and β-phenylalanoyl-CoA, respectively. The pH optimum for this recombinant O-(3-amino-3-phenylpropanoyl) transferase is at 6.8. Application of these transacylase sequences in suitable host cells can improve yields of Taxol® or can be used to potentially synthesize second-generation taxol analogs possessing greater bioactivity and water solubility. The progression of advanced metabolites in the Taxol® biosynthetic pathway is illustrated in FIG. 11A.

Substrates and Reagents

[³H]Baccatin III was prepared by the methods described in Taylor et al., J. Labelled Compd. Radiopharm. 33:501-515 (1993), except that the intermediate 7-triethylsilyl-[13-³H]baccatin III was deprotected with hydrogen fluoride by the methods described in Georg et al., Bioorg. Med. Chem. Lett. 4:335-338 (1994). N-tert-butoxycarbamates of(3RS)-β-phenylalanine and L-phenylalanine were prepared as described in Tarbell et al., Proc. Nat. Acad. Sci. USA, 69:730-732 (1972).

Coenzyme A as the lithium salt was purchased from Sigma-Aldrich Corp. (St. Louis, Mo.). Di-tert-butyl dicarbonate, benzoyl chloride, sodium hydride, β-phenylalanine (3-amino-3-phenylpropionic acid), L-phenylalanine, and N-benzoyl-(2R,3S)-3-phenylisoserine and all other reagents, unless otherwise noted, were purchased from Sigma-Aldrich Corp. Authentic baccatin III was generously provided by Hauser Chemical Research (Boulder, Colo.) or was synthesized from 10-deacetylbaccatin III (Natland; Morrisville, N.C.) as described in Cravallee et al., Tetrahedron Lett. 39:4263-4266 (1998). Authentic (3′RS)-2′-deoxytaxol was prepared from the corresponding N-debenzoylated analog by methods described in Georg et al., Bioorg. Med Chem. Lett., 4:335-338 (1994).

General Procedures

The general synthetic procedure for the synthesis of the amino acid CoA thioesters was adapted from procedures described in Rasmussen et al., Biochem. J. 265:849-855 (1990); Silva et al., Anal. Biochem. 290:60-67 (2001); and Walker, K. and Croteau, Proc. Natl. Acad Sci. USA 97:13591-96 (2000). This procedure utilized a mixed anhydride intermediate to facilitate trans-thioesterification of the acyl acceptor with CoA. In each case the lyophilized N-protected (and O-protected when necessary) amino acid-CoA ester, intermediary products were typically dissolved in 5-7 mL of resuspension buffer (15 mM potassium phosphate, pH 6.9) and purified by chromatography on a C₁₈ Sep-Pak cartridge (500 mg C₁₈ silica gel, Whatman) that was first washed with methanol (10 mL), water (10 mL), and then with resuspension buffer (10 mL). After loading, the product was eluted from the column with increasing methanol (5-100%) in resuspension buffer.

Synthesis of α- and β-Phenylalanoyl-CoA Esters.

The so derived N-butoxycarbonyl-α- or β-phenylalanoyl CoA, which eluted from the C₁₈ cartridge in 15-20% methanol, was lyophilized, and the remaining residue was dissolved in 1 mL of water, the solution was cooled to 0° C., and 1 mL of trifluoroacetic acid was added dropwise with stirring over 1 h. Then the mixture was warmed to room temperature and stirred for 1 h. The progress of deamidation of the N-tert-butoxycarbonyl compound was monitored by silica gel analytical TLC (1-butanol/H₂O/acetic acid 5:3:2, vol/vol/vol) with UV absorbance detection. The R_{f} values on TLC were at 0.6 and 0.3 for N-tert-butoxycarbonyl-α- and β-phenylalanoyl CoA and α- and β-phenylalanoyl CoA, respectively. After completion, the reaction was diluted with 50 mL of water, the mixture was concentrated to 0.5 mL under vacuum, and the dilution and evaporation process was repeated 3 times to remove trifluoroacetic acid. Finally, the sample was concentrated to dryness and the residue was resuspended in 5 mL of water. The product was purified by C₁₈ Sep-Pak cartridge chromatography as described previously. The CoA esters of α- or β-phenylalanine eluted in 10-20% methanol, the eluant was evaporated completely, and the remaining residue was resusupended in deuterated water as internal reference for analysis by ¹H-NMR spectrometry. The CoA ester was quantitated by comparing the peak area of assigned protons of the sample to that of the protons of dioxane (at 11 mM) added as an internal standard. (3RS)-β-Phenylalanoyl CoA was obtained at 40% yield (16 mmol at>95% purity based on ¹H-NMR) with respect to N-tert-butoxycarbonyl-(3RS)-β-phenylalanine (40 mmol), and S-α-phenylalanine was obtained at 26% yield (11 rmmol at>90% purity based on ¹H-NMR) with respect to N-tert-butoxycarbonyl-(S)-β-phenylalanine (42 mol). After NMR analysis, the D₂O was evaporated, and the CoA esters were dissolved in water to make a 10 mM solution. ¹H-NMR (300 MHz, D₂O) of (3RS)-β-phenylalanoyl CoA (see FIG. 11B for numbering) δ: 0.56 (s, H-10′), 0.70 (s, H-11′), 2.12 (dd, J=6.3 and 6.9 Hz, H-4′), 2.74 (m, H-1′), 3.04 (dd, J=5.1 and 6.0, H-2′), 3.21 (m, H-2 and H-5′), 3.37 (dd, J=4.8 and 9.6 Hz, Hₐ-5″), 3.65 (dd, J=4.8 and 9.6 Hz, H_{b}-5″), 3.85 (s, H-7′), 4.03 (d, J=3.8 Hz, Hₐ-9′), 4.05 (d, J=3.8 Hz, H_{b}-9′), 4.39 (ddd, J=2.7 and 5.3 Hz, H-4″), 4.56-4.66 (m, H-3, H-2″, and H-3″), 5.95 (two doublets; one set from each isomer, J=6.9 Hz for both, H-1″), 7.21-7.27 (phenyl protons), 8.01 (two singlets; one from each isomer, adenine-CH), and 8.33 (two singlets; one from each isomer, adenine-CH) and ¹H-NMR (300 MHz, D₂O) S-α-phenylalanoyl CoA (see FIG. 11B for numbering) δ: 0.54 (s, H-10′), 0.66 (s, H-11′), 2.21 (dd, J=6.3 and 6.6 Hz, H-4′), 2.82-2.95 (m, H-3 and H-1′), 3.02-3.11 (m, H-2 and H-2′), 3.23 (dd, J=6.3 and 6.9 Hz, H-5′), 3.34 (dd, J=4.8 and 9.6 Hz, Hₐ-5″), 3.60 (dd, J=4.8 and 9.6 Hz, H_{b}-5″), 3.80 (s, H-7′), 4.00 (d, J=4.2 Hz, Hₐ-9′), 4.02 (d, J=4.2 Hz, H_{b}-⁹′), 4.36 (br ddd, H-4″), 5.91 (d, J=6.6 Hz, H-1″), 6.96-7.19 (phenyl protons), 7.97 (s, adenine-CH), 8.30 (s, adenine-CH). H-2″ and H-3″ proton signals obscured by solvent (HOD) signal.

Synthesis of N-Benzoyl-(2R, 3S)-3-phenylisoseryl-CoA

To a solution of N-benzoyl-(2R,3S)-3-phenylisoserine (230 mg, 0.81 mmol) in 10 mL tetrahydrofuran was added (dropwise) excess diazomethane in diethylether. The solvents were evaporated and the product was purified by silica gel flash column chromatography (50:50, ethyl acetate/hexane, vol/vol) to yield pure N-benzoyl-(2R,3S)-3-phenylisoserine methyl ester (0.73 mmol, 90% yield). The methyl ester (330 mg, 0.73 mmol) was dissolved in tetrahydroftiran (15 mL), and the solution was added to a stirred suspension of sodium hydride (1.1 mmol) in diethylether (10 mL) under N₂. To the mixture was added di-tert-butyl dicarbonate (170 mg, 0.77 mmol) in terahydrofuran (20 mL), and the mixture was stirred at room temperature for 20 min. The mixture was chilled on ice, quenched with dropwise addition of 1 mL water, and then filtered through Celite. The filtrate was collected and the solvent evaporated. The product was purified by silica gel flash column chromatography (35:65, ethyl acetate/hexane, vol/vol) to yield pure N-benzoyl-O-tert-butoxycarbonyl-(2R,3S)-3-phenylisoserine methyl ester (0.66 mmol, 90% yield). To the O-protected methyl phenylisoserinate (26 mg, 65 [mol) in tetrahydrofuran (1.2 mL) was added NaOH (65 μmol, 32.5 μL of 2 M aqueous solution) and the solution was stirred for 12 h. The solvent was evaporated to yield the sodium salt of N-benzoyl-O-tert-butoxycarbonyl-(2R,3S)-3-phenylisoserine. The carboxylate sodium salt was susupended in tetrahydrofuran (1.4 mL) to which was added ethyl chloroformate (7.0 μL, 7.8 mg, 72 μmol) under N₂, and the mixture was stirred at room temperature for 1 h. The coupling of the N-benzoyl-O-tert-butoxycarbonyl-(2R,3S)-3-phenylisoserine anhydride with CoA, the purification of the intermediate N-tert-butoxycarbonyl-phenylpropanoyl CoA ester, and N-deprotection with trifluoroacetic acid was performed as described in the previous section. N-Benzoyl-(2R,3S)-3-phenylisoseryl-CoA eluted from C₁₈ cartridge in 15-20% methanol (20 mmol, 30%, yield based on the methyl-N-benzoyl-O-tert-butoxycarbonyl-3-phenylisoserinate (65 mmol)). The purity of N-benzoyl phenylisoseryl-CoA was judged by ¹H-NMR to be>95%. ¹H NMR (300 MHz, D₂O) for N-benzoyl phenylisoseryl-CoA (see FIG. 11B for numbering) δ: 0.49 (s, H-10′), 0.64 (s, H-11′), 2.05 (dd, J=6.3 and 6.6 Hz, H-4′), 2.77 (m, H-1′), 3.01 (dd, J=4.2 and 6.0 Hz, H-2′), 3.15 (dd, J=6.6 Hz, H-5′), 3.31 (dd, J=4.8 and 9.6 Hz, Hₐ-5″), 3.60 (dd, J=4.8 and 9.6 Hz, H_{b}-5″), 3,79 (s, H-7′), 4.02 (d, J=3.6 Hz, Hₐ-9′), 4.04 (d, J=3.6 Hz, H_{b}9′), 4.37 (br ddd, H-4″), 4.57-4.65 (m, H-2, H-2″, and H-3″), 5.42 (d, J=3.3 Hz, H-3), 5.93 (d, J=6.6 Hz, H-1″), 7.18-7.54 (phenyl protons), 7.97 (s, adenine-CH), and 8.30 (s, adenine-CH).

Synthesis of Phenylisoseryl-CoA

The synthesis of α- and β-phenylalanoyl-CoA esters via their acid labile N-tert-butoxycarbonyl protection intermediates proved to be productive routes to the corresponding free amino acid thioesters as described previously. Therefore, a synthesis of phenylisoseryl-CoA invoking an N-tert-butoxycarbonyl-phenylisoseryl methyl ester intermediate was pursued. A transamidation method was employed to convert N-benzoyl-phenylisoserine to N-tert-butoxycarbonyl-phenylisoserine, as described in Jagtap and Kingston, Tetrahedron Lett. 40:189-192 (1999). The details of this adapted procedure were performed as follows.

To methyl N-benzoyl-(2R,3S)-3-phenylisoserinate (470 mg, 1.6 mmol) (see above) in CH₂Cl₂/tetrahydrofuran (5:2, vol/vol, 28 mL) under N₂ were added diaminopyridine (370 mg, 1.7 mmol) in CH₂Cl₂ (1.6 mL) and benzyl chloroformate (244 μL, 290 mg, 1.7 mmol), and the mixture was stirred at room temperature for 1 h. The solvents were evaporated and product was purified by silica gel flash column chromatography (ethyl acetate/hexane, 35:65, vol/vol) to yield pure N-benzoyl-O-benzylformyl-(2R,3S)-3-phenylisoserine methyl ester (1.5 mmol, 90% yield).

To the methyl ester (1.5 mmol) dissolved in CH₃CN (10 mL) under N₂ were added 10 mL CH₃CN containing dimethylaminopyridine (780 mg, 3.6 mmol) and 20 mL of CH₃CN containing di-tert-butyl dicarbonate (3.5 g, 16 mmol), and the mixture was stirred for 24 h at room temperature. The solvent was evaporated and the residue was dissolved in 15 mL CH₃CN and 100 mL of ethyl acetate. The organic layer was washed with brine, dried over Na₂SO₄, and evaporated under vacuum. The product was purified by silica gel flash column chromatography (25:75, ethyl acetate/hexane, vol/vol) to yield pure methyl N-benzoyl-N-tert-butoxycarbonyl-O-benzyloxycarbonyl-(2R,3S)-3-phenylisoserinoate (0.3 mmol, 20% yield). To the methyl phenylisoserinate (0.3 mmol) in 20 mL methanol was added 20 mL 6% magnesium methoxide methanol solution. The mixture was stirred for 1 h at room temperature and diluted with 300 mL of ethyl acetate. The organic layer was washed with brine, water, again with brine, dried over Na₂SO₄, and then evaporated. The product was purified by silica gel flash chromatography (15-35% ethyl acetate gradient in hexane) to yield methyl N-tert-butoxycarbonyl-(2R,3S)-3-phenylisoserinate (0.24 mmol, 80% yield). Protection of hydroxyl group of the methyl ester was achieved by treatment with sodium hydride then di-tert-butyl dicarbonate as described in the previous section. The yield of methyl N,O-bis-(tert-butoxycarbonyl)-(2R,3S)-3-phenylisoserinate was 0.2 mmol (80% yield). The hydrolysis of N,O-bis-(tert-butoxycarbonyl)-(2R,3S)-3-phenylisoserine methyl ester with NaOH, esterification of the liberated carboxylate with CoA, purification of the target CoA ester, and elimination of tert-butoxycarbonyl group with trifluoroacetic acid were carried out as described herein. (2R,3S)-phenylisoserinoyl-CoA, which eluted from the C₁₈ cartridge in 10% methanol (2.9 μmol, 10% yield based on the methyl N,O-bis-(tert-butoxycarbonyl)-3-phenylisoserinate (30 μmol). The purity of phenylisoserine-CoA was judged by ¹H-NMR to be ˜80%. ¹H NMR (300 MHz, CD₃OD) for phenylisoseryl-CoA (cf. FIG. 11B for numbering) δ: 0.83 (s, H-10′), 1.05 (s, H-11′), 2.44 (dd, J=6.5 and 6.7 Hz, H-4′), 2.79 (m, H-1′), 3.45 (m, H-2′), 3.47 (dd, J=6.6 Hz, H-5′), 3.57 (dd, J=6.6 and 10.5 Hz, Hₐ-5″), 3.98 (dd, J=5.4 and 9.9 Hz, H_{b}-5″), 4.06 (s, H-7′), 4.23 (d, J=3.8 Hz, Hₐ-9′), 4.25 (d, J=3.8 Hz, H_{b}-9′), 4.49 (br ddd, H-4″), 4.69-4.90 (m, H-2, H-3, H-2″, and H-3″), 6.13 (d, J=6.0 Hz, H-1″), 7.25-7.41 (phenyl protons), 8.18 (s, adenine-CH), and 8.57 (s, adenine-CH).

Heterologous Expression, Functional Transferase Assay, and Product Analysis

The cloning and expression of Taxus transacylase cDNAs and expression of the corresponding polypeptides are described herein. A 0.1 mL aliquot (˜0.5 mg total protein) of each soluble enzyme preparation was incubated for 3 h at 31 ° C. with 100 gM of the acyl-CoA co-substrate and 70 μM (0.5 μCi) [13-³H]baccatin III. The reaction mixture was basified to pH≈9 with saturated sodium bicarbonate solution and treated with benzoyl chloride (7 μmol) for 0.5 h at 25° C. After Schotten-Baumann derivatization, the mixture was extracted as described in Walker, K. & Croteau, R., Proc. Natl. Acad. Sci. USA 97:13591-96 (2000), and analyzed by radio-HPLC [Perkin-Elmer (Shelton, Conn.) HPLC ISS 200 pump coupled to a 25 Perkin-Elmer ABI 785A UV/Visible Detector and a Packard (Meridian, Conn.) A-100 Radiomatic detector]. The samples were separated on a Phenomenex (Torrance, Calif.) reverse-phase Phenyl-3 column (5 μm, 4.6×250 mm) by elution at 1 mL/min with 25:75 (v/v) CH₃CN:H₂O for 5 min, then to 65:35 (v/v) CH₃CN:H₂O with a linear gradient over 40 min, then ramped to 85:15 (v/v) CH₃CN:H₂O and held 5 min, and finally returned to initial conditions.

Of the enzyme preparations evaluated, only that from an E. coli transformant bearing the clone designated TAX7 (when tested with all co-substrates) generated a single radioactive product (with a coincident absorbance at 254 nm) with a retention time identical to that for authentic (3′RS)-2′-deoxytaxol.

Sufficient enzyme was obtained from large-scale cultures of E. coli transformed with TAX7 for preparative conversion of substrate to product for characterization. The resulting N-debenzoylated product (˜1 mg) was chemically benzoylated as before, extracted twice with ether, the solvent evaporated, and the crude product mixture was chromatographed by TLC (0.5 mm silica gel, 50:50 (v/v) EtOAc:hexane). The material that co-migrated with authentic (3′RS)-2′-deoxytaxol R_{f}=0.15) was analyzed by ¹H-NMR (Varian Mercury 300 instrument) and combined liquid chromatography mass spectrometry on a Hewlett-Packard Series 1100 MSD system in the atmospheric pressure chemical ionization mode. The sample was dissolved in acetonitrile, loaded onto a Supelcosil Discovery HS F5 column (5μ, 4.6×250 mm, Sigma-Aldrich Corp., St. Louis, Mo.), and eluted with 50:50 (v/v) acetonitrile:water at 1 mL/min, with the effluent directed to the atmospheric pressure chemical ionization mass detector in the positive-ion mode.

Partial Purification and Characterization of Recombinant T. cuspidata Phenylpropanoyltransferase

Attempts to overexpress operationally soluble TAX7 polypeptide from pSBET in E. coli resulted in the formation of inclusion bodies and aggregates. The phenylpropanoyltransferase expressed to levels of about<1% (as determined by SDS-PAGE) of the total soluble bacterial protein. The enzyme activity was further compromised by partial purification of the crude soluble extract on a diethylaminoethyl-cellulose (Whatman DE-52, Clifton, N.J.) column (2.5×20 cm; 75 mL bed volume) that was previously equilibrated with 50 mM Mopso (pH 7.2) containing 5% glycerol, 5 mM MgCl₂, and 0.5 mM dithiothreitol (Buffer A). After washing with three column volumes of Buffer A, elution of the target enzyme was achieved with a linear NaCl gradient (0-500 mM; 500 mL; 10 ml/min). Recovered activity was 15-20% of the total loaded before chromatography, presumably due to the instablity of the enzyme. Therefore, the crude preparation was used as the enzyme source to determine kinetic parameters, and in preparative assays to generate sufficient product for confirmation by spectrometric methods.

After determining optimal protein concentration and reaction time for calculating kinetic parameters, standard assays were performed at reciprocally varied co-substrate concentrations (0-1 mM) with the remaining reactant at saturation (1 mM). KALEIDAGRAPH (version 3.08, Synergy Software, Reading, Pa.) was used for calculations with double reciprocal plotting of each data set, and the equation for the best fit line (R²=0.99) was determined. The pH optimum for this O-acyltransferase was assessed in assay mixtures containing 0.44 mg of the partially purified enzyme, each diluted with 200 μL buffer comprising either 25 mM sodium acetate (pH 4.6), 2-(N-morpholino) ethanesulfaric acid (Mes) (pH 5.6-7.0, potassium phosphate (pH 6.2-7.4), (N, N-bis [2-hydroxyethyl]-glycine (Bicine) (pH 7.6-9.2).

Cloning and Expression

The biosynthetic origin of the Taxol® C-13 side chain is proposed to arise from phenyalanine, which is converted, via β-phenylalanine, to the free amine of phenylisoserine. Stable-isotope feeding studies have demonstrated that the latter 3-amino-2-hydroxy-3-phenylpropanoate was incorporated into Taxol®, but with lower efficiency than was β-phenylalanine. Benzamidation of N-debenzoyltaxol (i.e., the phenylisoserine baccatin III ester) is considered to be the last step in Taxol® biosynthesis.

Evaluation of the remaining clones of the extant group required the synthesis of β-phenylalanoyl-CoA ester (and other possible candidate CoA esters) and [³H]baccatin III as co-substrates. There are several enzymatic methods described for small-scale and large-scale preparation of coenzyme-A thioesters. Typically, radioactive coenzyme A esters are biosynthesized at high specific activity for preliminary screening of enzyme function. When larger quantities of unlabeled acyl CoA-derivatives are needed for preparative applications, a chemical synthesis approach is more practical. A large-scale enzymatic preparation of benzoyl-CoA analogs has been described in Myers and Utter, Anal. Biochem. 112:23-29 (1981), but this approach requires lengthy incubations, and the excess reaction cofactors are difficult to separate from the target acyl-CoA. Therefore, a chemical synthesis procedure, employing a mixed anhydride intermediate, was chosen to prepare β-phenylalanoyl-CoA as a potential co-substrate for screening for the transferase. In brief, β-phenylalanine was converted to N-t-Boc-β-phenylalanine by methods described in Tarbell et al., Proc. Nat. Acad. Sci USA 69:730-732 (1972), and esterified with coenzyme A using procedures described in Rasmussen et al., 1990; Silva 2001; and Walker and Croteau, 2000.

Facile removal of the t-Boc group by trifluoroacetic acid treatment followed by rapid purification of the product by reversed-phase C₁₈ chromatography afforded β-phenylalanoyl-CoA in high yield and purity. Coenzyme A esters of α-phenylalanine, phenylisoserine and N-benzoyl phenylisoserine were similarly prepared except that the latter two amino acids required t-Boc protection of the C-2 hydroxyl before conversion to the anhydride. [³H]Baccatin III was prepared by modification to the procedure of Taylor et al., J. Labelled Compd. Radiopharm. 33:501-515 (1993).

The five remaining full-length Taxus transacylase nucleic acids from the family of nine such cDNA clones were assessed. Using appropriate primers (Table 3) and a sticky-end PCR method (Zeng, BioTechniques 25, 206-208 (1998))., these cDNAs were transferred from the previously employed pCWori+ vector (5) to pSBET (Schenk, P.M., et al., BioTechniques 19, 196-200 (1995)). Denaturating and reannealing of the derived amplicon mixtures yielded cohesive-end products for each clone that contained 5′-NdeI and 3′-BamHl terminal overhangs to permit directional ligation into the appropriately digested pSBETa vector. These sequence verified vector constructs were individually transformed into E. coli BL21 (DE3) for expression. Each culture harboring a unique transacylase was used to inoculate 100 mL of Luria-Bertani medium supplemented with kanamycin (50 μg/mL) and was grown at 37° C. to an A₆₀₀=1.0, and then induced by addition of isopropyl-β-D-thiogalactopyranoside to a final concentration of 0.5 mM. The cultures were then incubated at 18° C. with shaking (220 rpm) for 16 h, harvested by centrifugation (2000 g, 20 min), resuspended in 10 mL extraction buffer [50 mM Mopso (pH 7.2) 5% glycerol, 1 mM EDTA, 0.5 mM dithiothreitol], and then disrupted by sonication for 30 s at 0° C. using a Virsonic 475 (Virtis, Gardiner, N.Y.) with a 1.5 cm probe at maximum power. The resulting homogenates were clarified by centrifugation (45,000 g, 1 h) to provide the soluble enzyme fraction. A 1 mL aliquot (5 mg total protein) of the soluble enzyme preparation was incubated with 100 μM of each synthesized amino phenylpropanoyl-CoA and 70 μM (0.5 μCi) of [13-³H]baccatin as co-substrates for 3 h at 31° C.

After incubation for 3 h at 31° C., each enzyme preparation (except where N-benzoyl phenylisoseryl-CoA was used as co-substrate) was subjected to the Schotten-Baumann method using benzoyl chloride as the acyl donor to selectively N-benzoylate any biosynthesized amino phenylpropanoyl baccatin III putatively produced in the assay; this N-derivatization allowed selective, organic extraction of product and chromatography on reversed-phase HPLC.

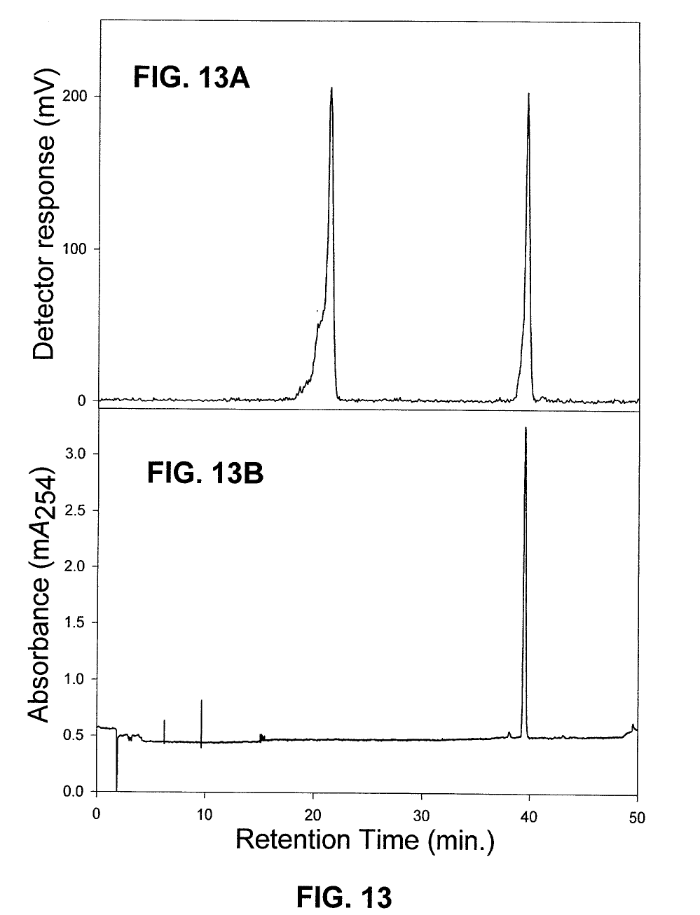

Only recombinant enzyme (SEQ ID NO: 52) expressed from clone TAX7 (SEQ ID NO: 52) catalyzed the formation of a product that (after chemical N-benzoylation) chromatographed on radio-HPLC with exactly the same retention time (39.6±0.1 min) as that of authentic (3′RS)-2′-deoxytaxol (FIGS. 13A-13B). No product was detected in assays without either co-substrate or in control assays with boiled enzyme in the presence of both co-substrates at apparent saturation, or in complete assays in which the chemical benzamidation step was excluded.

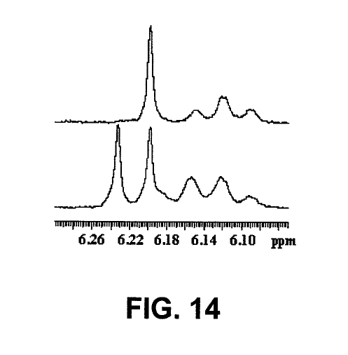

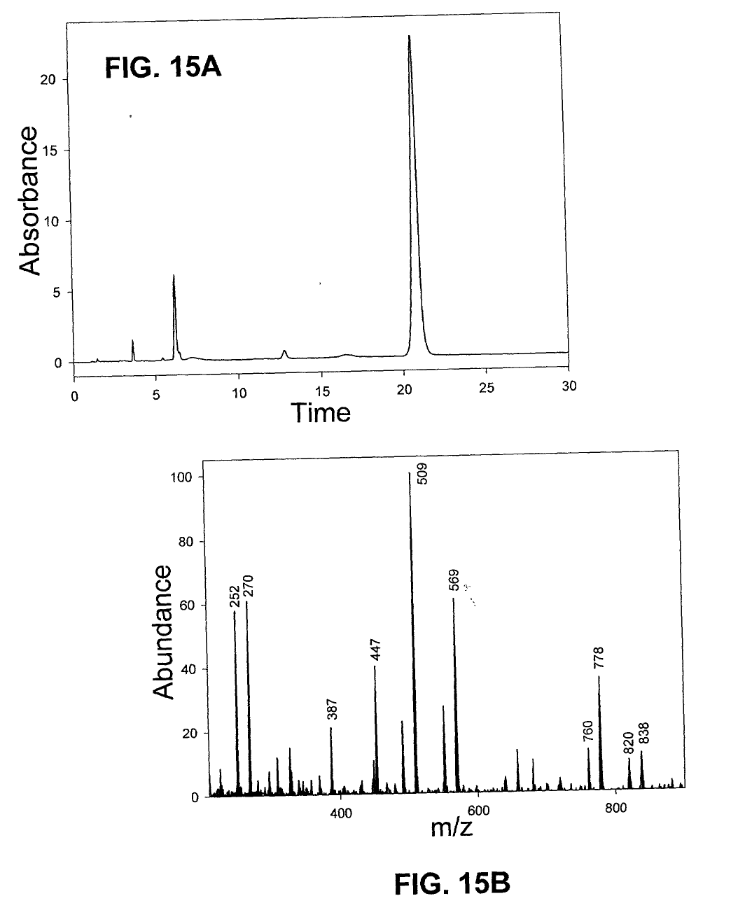

The molecular weight of the TAX7 encoded enzyme was calculated to be 50,546 Daltons, which is similar in size to the 50 kDa soluble recombinant TAX7 enzyme (assessed by SDS/PAGE). Partially purified enzyme (by anion-exchange and hydroxyapatite chromatography) was used for preparative-scale conversion of the co-substrates to the putative β-phenylalanoyl baccatin III ester which was N-benzoylated as before. The product was purified by TLC (99% pure by UV-HPLC) and analyzed by ¹H-NMR. The sample was judged to contain a single 2′-deoxytaxol 3′-epimer (FIG. 14) by comparison to a ¹H-NMR spectrum of an authentic (3′RS)-2′-deoxytaxol standard. The identity of the benzoylated product was also confirmed by atmospheric pressure chemical ionization mass spectrometry (ACPI-MS) and comparison of the mass spectrum to that of the authentic standard (FIGS. 15A-B). Taken together, these spectrometric data confirm that TAX7 enzyme encodes a baccatin III 13-O-(3-amino-3-phenylpropanoyl)transferase.

Functional Characterization of Recombinant Phenylpropanoyltransferase

The calculated Kₘ values (Lineweaver-Burk plotting (R²=0.99)) for the recombinant enzyme designated BAPT are 2.6 μM and 4.5 μM for baccatin III and β-phenylalanoyl-CoA, respectively. The pH optimum was found to be at pH 6.8. This enzyme catalyzed the regiospecific acylation at the C-13 hydroxyl of baccatin III (no transfer to C-1 or C-7 was observed), and exhibits selectivity for 3-amino-3-phenylpropanoyl-CoA esters with a preference for β-phenylalanoyl-CoA (Vᵣₑₗ=1.0) over 3-phenylisoseryl-CoA (Vᵣₑₗ=˜0.5); α-phenylalanoyl-C and N-benzoyl phenylisoseryl-CoA were not productive acyl donors (Vᵣₑₗ<0.001).

Sequence Analysis

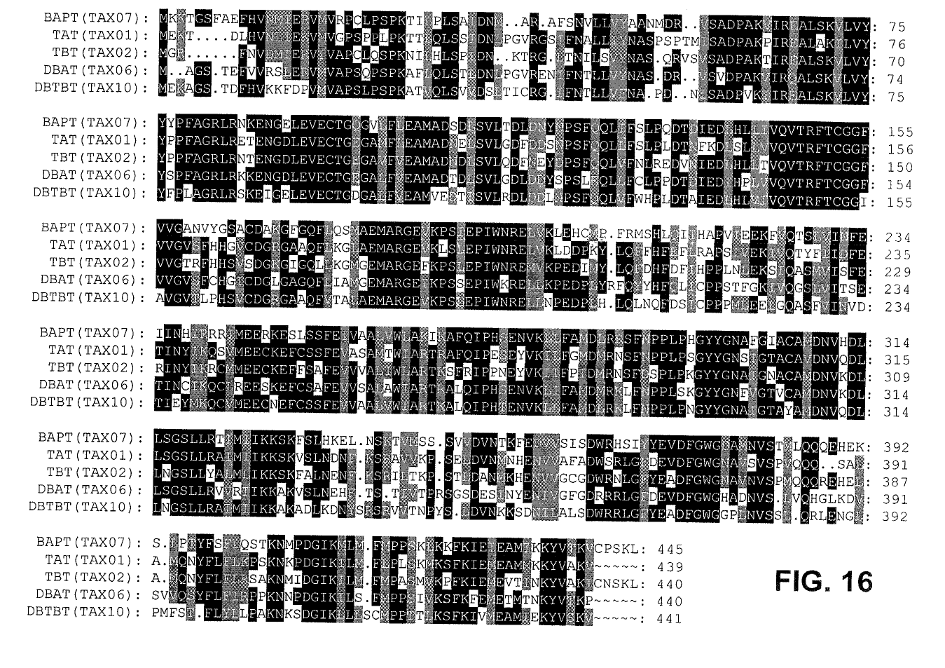

BAPT (GenBank accession no. AY082804) is a 1,335 nucleotide encoding a 445 amino acid polypeptide with a calculated molecular weight of 50,546. The peptide similarity amongst the five acyl/aroyltransferases involved directly in Taxol® biosynthesis is between 71-74% (FIG. 16). The BAPT sequence, however, contains a ¹⁶³GXXXDA¹⁶⁸ instead of the typical acyltransferase HXXXDG motif (also occurring, though less frequently, as HXXXDA) of which the His and Asp along with a conserved upstream cysteine residue (Cys95) (FIG. 16) are suggested to form a catalytic triad (Cys, His, Asp) for acyl group transfer catalysis. Conceivably, the Gly163 for His163 substitution in BAPT could disrupt the catalytic triad function; however, the free β-amine of the CoA ester substrate could, through hydrogen bonding, function as an intramolecular catalytic general base/acid in place of the normal histidine.

### Example 4

### Characterization of a benzoyl-CoA:3′-N-debenzoyl-2′-deoxytaxol N-benzoyltransferase

The TAX10 cDNA clone (SEQ ID NO: 53) was isolated as described in Example 1. This example describes how the polypeptide expressed from the cDNA (SEQ ID NO: 54) was found to encode a 3′-N-debenzoyl-2′-deoxytaxol N-benzoyltransferase.

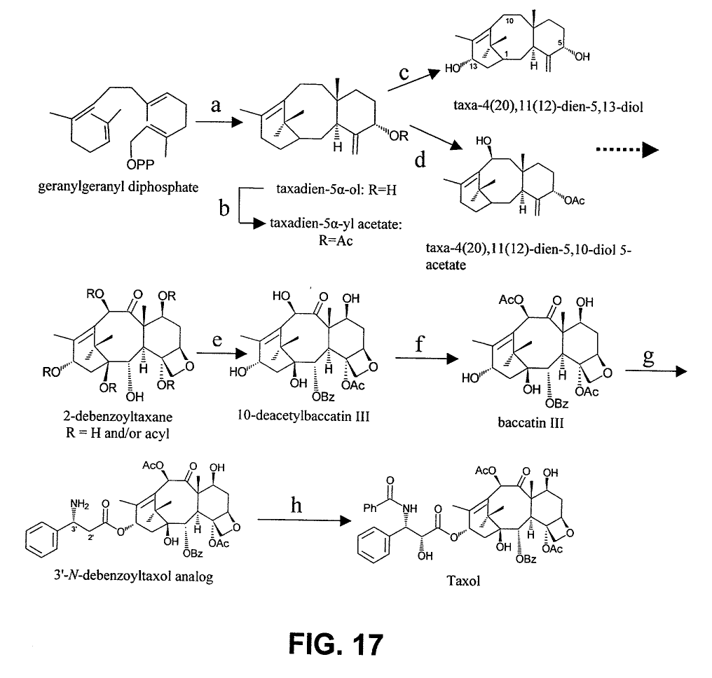

This enzyme catalyzes the stereoselective coupling of the semisynthetic surrogate substrate (3′RS)-N-debenzoyl-2′-deoxytaxol with benzoyl-CoA to form (3′RS)-2′-deoxytaxol, and, therefore, this enzymatic amidation defines the final acylation in the Taxol® biosynthetic pathway (FIG. 17). The product, (3′RS)-2′-deoxytaxol, was confirmed by radio-HPLC, ¹H-NMR, and direct-injection chemical ionization-MS. The full-length cDNA has an open reading frame of 1,323 base pairs and encodes a 441 amino acid protein with a calculated molecular weight of 49,040 Da. The recombinant N-benzoyltransferase has a pH optimum at 8.0, a k_{cat}≈1.5±0.3 s⁻¹, Kₘ values of 0.42 mM and 0.40 mM for the N-deacylated taxoid and benzoyl-CoA, respectively, and a Vₘₐₓ at ˜7.8 pkat. In addition to this enzyme aiding to increase the production of Taxol® in genetically engineered host systems, it also provides a means to attach a benzoyl (or modified aroyl) group to several N-dearoyl Taxol® analog precursors for the purpose of improving cytotoxicity.

Substrates

(3′RS)-N-Debenzoyl-2′-deoxytaxol and (2′S)-13-O-α-phenylalanylbaccatin III were similarly synthesized as described in Shiina et al., Bull. Chem. Soc. Jpn. 73:2811-18 (2000) and Saitoh et al., Chem. Lett. 7:679-80 (1998), except that either N-Boc-(3RS)-β-phenylalanine or N-Boc-(3S)-α-phenylalanine (Aldrich) was coupled to 7-TES-baccatin III. Deprotection by methods described in Georg et al., Bioorg. Med. Chem. Lett. 3:2467-70 (1993) yielded the desired substrates. [7-¹⁴C]Benzoyl-CoA was prepared by the methods described in Walker and Croteau, Proc. Natl. Acad. Sci. USA 97:13591-96 (2000). Other coenzyme A thioester salts and (3RS)-β-phenylalanine (3-amino-3-phenylpropionic acid) were purchased from Sigma-Aldrich (St. Louis, Mo.). Authentic baccatin III was generously provided by Hauser Chemical Research (Boulder, Colo.) or synthesized from 10-deacetylbaccatin III as described in Cravallee et al., Tetrahedron Lett. 39:4263-4266 (1998). Synthesis of (3′RS)-2′-deoxytaxol from the (3′RS)-N-debenzoyl substrate was prepared by methods described in Georg, G. I., et al., 1993.

Bacterial Expression, N-Benzoyltransferase Assay, and Product Identification

The isolation of nine transacylases clones involved in Taxol® biosynthesis is described herein. A sticky-end PCR method (see Zeng, BioTechniques 25:206-208 (1998)) was used to modify these cDNAs, previously in pCWori+ (see Walker et al., Arch. Biochem. Biophys. 374:371-380 (2000)) with appropriate primer pairs. Denaturating and reannealing of the respective amplicon mixtures yielded suitable cohesive-end products for each clone, containing 5′-NdeI and 3′-BamHl overhangs at the termini that were directionally ligated into separate NdeI/BamHI-digested pSBETa vectors; these recombinant vectors were used individually to transform E. coli BL21(DE3).

For enzyme expression, E. coli cultures transformed with a pSBET vector that harbored a putative transacylase nucleic acid were used to inoculate 100 mL of Luria-Bertani medium supplemented with kanamycin (50 μg/mL) and grown at 37° C. to an OD₂₆₀=1.0, whereupon expression was induced by addition of isopropyl-β-D-thiogalactopyranoside to a final concentration of 0.5 mM. After induction, the cultures were shaken and incubated at 18° C. for 16 h. Bacteria were harvested by a 20 min, low speed centrifugation (2000 g), resuspended in 10 mL extraction buffer (50 mM Mopso, pH 7.2, 5% glycerol, 1 mM EDTA, 0.5 mM dithiothreitol), and disrupted by sonication for 30 s at 0° C. using a Virsonic 475 (Virtis, Gardiner, N.Y.) with a 1.5 cm probe at maximum power output. Sonicates were clarified by centrifugation at 45,000 g for 1 h to provide the soluble enzyme fraction.

An aliquot (1 mL) of the soluble enzyme preparation was incubated with (3′RS)-N-debenzoyl-2′-deoxytaxol (100 μM) and [7-¹⁴C]benzoyl-CoA (80 μM, 1.5 μCi) for 2 h at 31 ° C. The reaction mixture was worked-up as described in Walker and Croteau, 2000, and the organic extracts were analyzed by radio-HPLC (Perkin-Elmer (Shelton, Conn.) HPLC ISS 200 pump coupled to a Perkin-Elmer ABI 785A UV/Visible Detector and a Packard (Meridian, Conn.) A-100 Radiomatic detector). The samples were separated on a Phenomenex, Inc. (Torrance, Calif.) reverse-phase Phenyl-3 column (5 μm, 4.6×250 mm) with elution at 1 mL/min with 25:75 CH₃CN/H₂O for 5 min, then to 65:35 CH₃CN/H₂O with a linear gradient over 40 min, ramped to 85:15 CH₃CN/H₂O and held 5 min, and finally returned to initial conditions. The soluble enzyme extract from an E. coli transformant expressing the clone (designated TAX10; SEQ ID NO: 53) generated a product detected at 254 nm and coincident with a radioactivity response at the same retention time as authentic (3′RS)-2′-deoxytaxol, which elutes as a single peak.

The described sticky-end PCR method was used to modify this clone with primers TX10NDE1F (5′-TGG AGA AGG CAG GCT CAA CAG-3′) paired with TX10BAM1R (5′-GAT CCT CAC ACT TTA CTT ACA TAT TTC TC-3′) and TX10NDE2F (5′-TAT GGA GAA GGC AGG CTC AAC AG paired with TX10BAM2R (5′-CTC ACA CTT TAC TTA CAT ATT TCT C-3′). All four primers were derived from SEQ ID NO. 53. The derived cohesive-end product was directionally subdloned into pSBETa, which was used to transform E. coli BL21(DE3). The complete sequence of the TAX10 insert was confirmed by comparing overlapping forward- and reverse-primed sequence of the appropriate strand.

This TAX10 transformant was cultured in large-scale (4 liters) to express enzyme for preparative conversion of product for NMR analysis. Product (˜1 mg) generated by large-scale preparation of the putative N-benzoyltransferase was purified by preparative silica gel TLC (0.5 mm, 50:50 EtOAc/hexane, v/v), and the band co-migrating with authentic (3′RS)-2′-deoxytaxol (R_{f}=0.15) was isolated, dissolved in 0.75 mL CDCl₃ as internal standard, and analyzed by proton nuclear magnetic resonance spectroscopy (¹H NMR) using a Varian Mercury 300 instrument. A fraction (˜30 μg) of the TLC-purified product was dissolved in 5 mL methanol and, by syringe-pump delivery, injected directly into an atmospheric pressure chemical ionization (APCI) probe linked to an LCQ (ThermoQuest/Finnigan; San Jose, Calif.) ion-trap mass detector instrument in positive-ion mode.

Partial Purification and Characterization of Recombinant T. cuspidata N-Benzoyltransferase

Four liters of E. coli BL21(DE3) cultures transformed with TAX10 were grown in 2.8 liter Fembach incubation flasks, harvested and extracted. The soluble extract was analyzed by SDS-PAGE and Coomassie Blue staining and demonstrated to possess an overexpressed protein of appropriate size (˜50 kDa) when compared to soluble extract of E. coli transformed with empty vector. The extract (150 mL) was loaded onto a diethylaminoethyl-cellulose (Whatman DE-52, Clifton, N.J.) column (2.5×20 cm; 75 mL bed volume) equilibrated with 50 mM Mopso, pH 7.2, containing 5% glycerol, 5 mM MgCl₂, and 0.5 mM dithiothreitol (Buffer A). After washing with three column volumes of Buffer A, elution of the enzyme was achieved with a linear NaCl gradient (0-500 mM; 500 mL; 10 mL/min). Fractions containing the bulk of the recombinant protein (120-200 mM NaCl, ˜80 mL) were pooled and loaded onto a 40 μm Type 1 Ceramic Hydroxyapatite (BioRad, Hercules, Calif.) column (2.5×20 cm; 50 mL bed volume) previously equilibrated with Buffer B (Buffer A containing 150 mM NaCl). After washing with three column volumes of Buffer B, proteins were eluted with a linear gradient of 0-100 mM potassium phosphate, pH 7.2 (250 mL; 10 mL/min). Fractions containing N-benzoyltransferase activity, eluting between 25-50 mM phosphate, were pooled, concentrated to 5 mg/mL (˜10 mL) using an Amicon Centriprep YM-30 centrifugal concentrator (Millipore, San Jose, Calif.), and filtered through a 0.45 μm Acrodisc syringe filter (Pall Life Sciences, Ann Arbor, Mich.). The filtrate containing partially purified protein (˜75% pure) was flash-frozen (liquid N₂) in 100 μL batches and stored at −80 for subsequent characterization studies.

After determining linearity with respect to protein concentration and time, k_{cat}, Kₘ, and Vₘₐₓ were determined using standard assay conditions at reciprocally varied cosubstrate concentrations (0-1 mM) with the remaining reactant at saturation (1 mM). KALEIDAGRAPH (version 3.08, Synergy Software, Reading, Pa.) was used for calculations, with double-reciprocal plots for each data set, and the equation for the best fit line (R²=0.99) was determined. The data are reported as the mean of duplicate assays. The pH optimum for N-acyltransferase activity was assessed in assays containing 5 μl of partially purified enzyme (˜0.5 μg protein) each diluted with either 78 μL 25 mM sodium phosphate (at pH 5.5-9.0) or 3-[cyclohexylamino]-2-hydroxy-1-propanesulfonic acid (Capso) (at pH 9.5-10) buffers.

Cloning and Heterologous Expression of a 3′-N-Debenzoyl-2′-Deoxytaxol N-Benzoyltransferase

The syntheses of N-debenzoyltaxols have been previously described (see, e.g., Gunatilaka et al., J. Org. Chem. 62, 3775-3778 (1997) and Georg, 1993), but the methods require multiple steps and/or use of costly, precious natural products (for example, Taxol® or cephalomannine) as starting material. Therefore, a direct, high-yielding four-step synthesis was implemented to couple 7-TES-baccatin III to a phenylpropanoid, (3RS)-N-Boc-β-phenylalanine, requiring minimal functional group protection. Deprotection afforded 3′RS-N-debenzoyl-2′-deoxytaxol as a surrogate substrate for surveying the expressed acyltransferases in cell-free assays.

The six full-length Taxus cDNA clones, originally in pCWori+, were transferred by a cohesive-end PCR method into pSBETa plasmid containing a T7 promoter and the argU nucleic acid which encodes for the tRNA that allows for rare (in E. coli) plant arginine codons AGA and AGG. Three Taxus nucleic acids encoding acyltransferases of known function (TAX1, TAX2, TAX6) were similarly subdloned into pSBET and used as expression vector negative controls. Each pSBET construct was used to individually transform E. coli BL21(DE3), and semipreparative cultures of each transformed and induced bacterium were generated. The cells were harvested, extracted, and the soluble fraction clarified for use in assays under standard conditions described in Walker, K. & Croteau, R. (2000), with (3′RS)-N-debenzoyl-2′-deoxytaxol and [7-¹⁴C]benzoyl-CoA as cosubstrates.

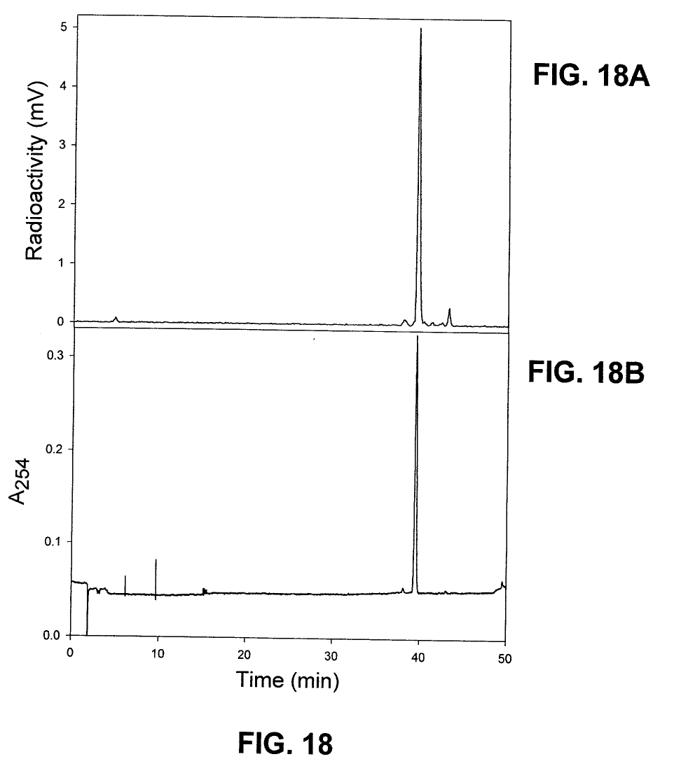

Enzyme expressed from the cDNA designated TAX10 (SEQ ID NO: 53) yielded a biosynthetic product that was revealed by reverse-phase radio-HPLC analysis (as described herein) to possess a retention time of 39.6±0.1 min (with coincident radio and UV traces) corresponding exactly to that of authentic (3′RS)-2′-deoxytaxol (FIG. 18); the HPLC conditions used did not resolve the diastereoisomers. Control extracts of E. coli host cells transformed with TAX1, TAX2, or TAX6 of defined function did not yield detectable product when assayed by identical methods. Additionally, no substrate conversion was detected in enzyme assays where either cosubstrate was absent nor in control assays with boiled protein in the presence of both cosubstrates at saturation.

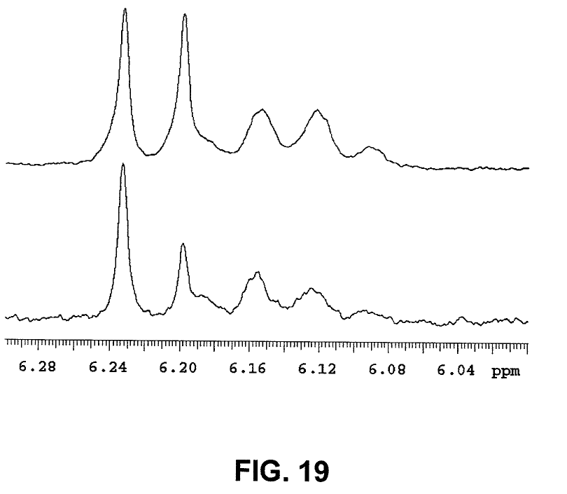

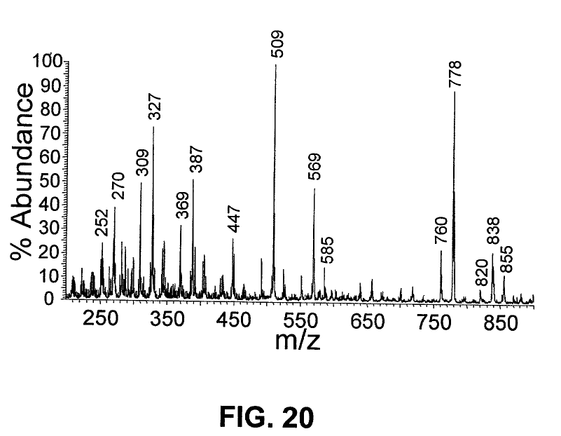

The TAX10 nucleic acid was overexpressed as a ˜50 kDa protein (determined by SDS/PAGE) from pSBET in induced E. coli BL21(DE3). This operationally soluble recombinant enzyme was expressed in large-scale (4 liter) culture of transformed bacteria. The extracted enzyme was partially purified sequentially by strong anion-exchange (DE-52) and ceramic hydroxyapatite chromatographies and used in sufficient quantities to generate ˜1 mg of sample that was purified by preparative silica gel TLC. ¹H-NMR analysis of authentic (3′RS)-2′-deoxytaxol showed a diastereoisomeric ratio at 1:1 after comparing the integration of the diagnostic H-10 signal that appears as a singlet (δ6.196 and 6.232) for each isomer (FIG. 19). A similar analysis of the N-benzoyltransferase-generated product revealed that DBTNBT is stereoselective for production of one isomer in 40% excess (FIG. 19). The absolute stereochemistry of each product isomer has not been established, but the enzyme product mixture likely contains excess 3′R-isomer, consistent with the Taxol® stereochemistry at this position. Further analysis by atmospheric pressure chemical ionization mass spectrometry (APCI-MS) (FIG. 20) revealed that the enzymatic product possesses an identical mass spectrum as the authentic standard.

Recombinant N-Benzoyltransferase Characterization

The pH optimum for the recombinant DBTNBT enzyme was found to be 8.0, with half maximal velocities near pH 7.0 and 9.0. This pH optimum is similar to those of already defined Taxol® pathway acyltransferases and other acyltransferases of plant origin.

Kₘ values of 0.45 mnM and 0.41 mM were calculated for N-debenzoyl-2′-deoxytaxol and benzoyl-CoA, respectively, by double-reciprocal plot analysis of both substrates (R²=0.99). Vₘₐₓ and k_{cat} were calculated to be ˜7.8 pkat and 1.5±0.3 s⁻¹, respectively. The DBTNBT is apparently regiospecific for acylation at the 3′-amino group on 13-O-β-phenylalanyltaxane substrates but not at the 2′-amino group as evidenced by the lack of observable amide formed when 13-O-α-phenylalanylbaccatin III and [7-¹⁴C]benzoyl-CoA were used as cosubstrates. The free amino acid β-phenylalanine was also not a productive substrate, suggesting that this enzyme requires 3-amino phenylpropanic acid as its baccatin ester for N-acyl group transfer. Additionally, enzymatic amidation of S-α-phenylalanine was not observed.

To evaluate the relative selectivity of the transferase for the acyl donor, benzoyl-CoA was compared to phenylacetyl-CoA and acetyl-CoA esters as cosubstrates at saturation. Evaluation of Vᵣₑₗ shows that benzoyl-CoA (100%) is the comparatively superior acyl donor than acetyl-CoA (1%) and phenylacetyl-CoA (1%).

Sequence Analysis

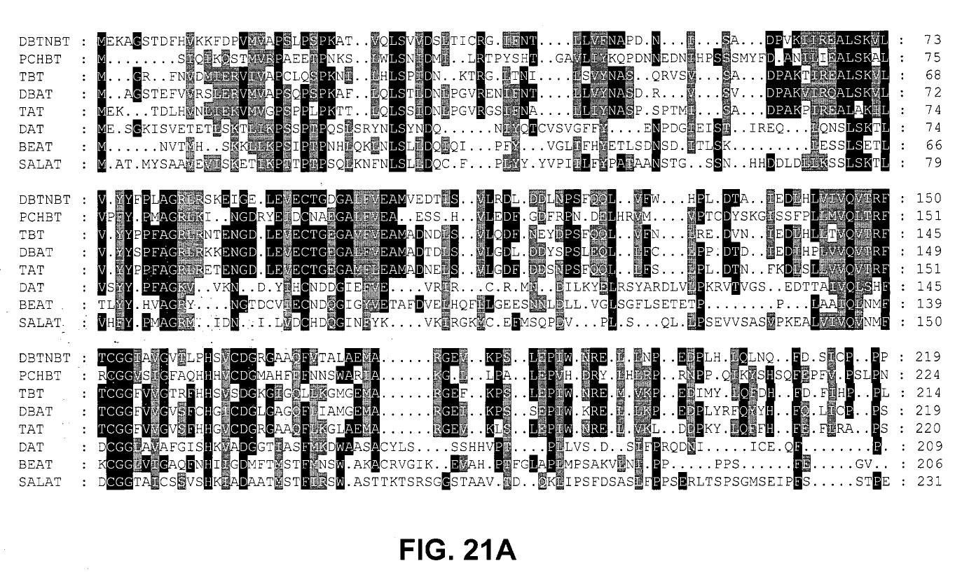

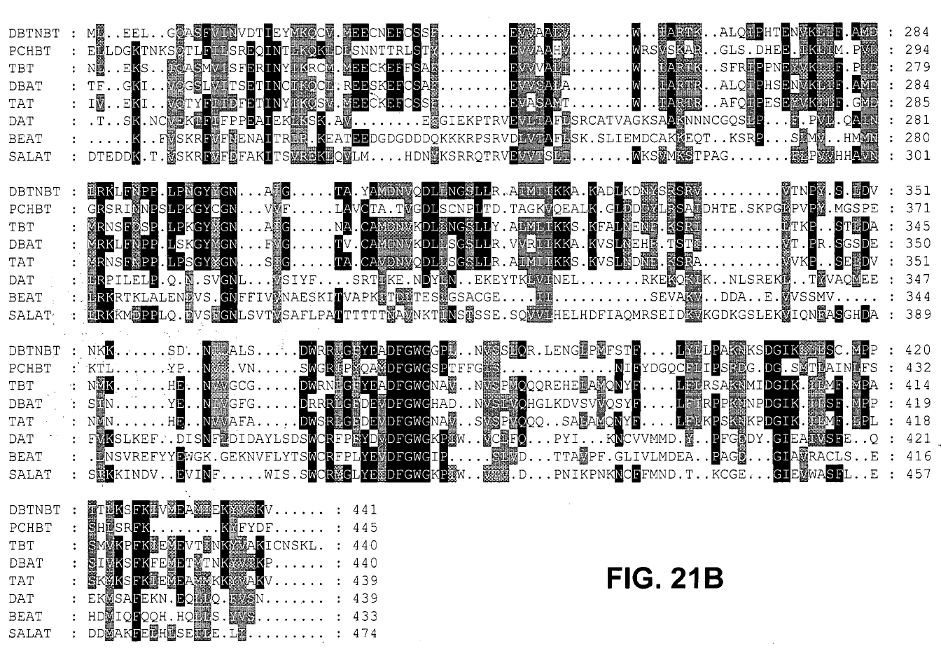

Sequence information for the DBTNBT cDNA and encoded peptide sequence can be obtained from the GenBank database (accession no. AF466397). The translated 1,323 nucleotide DBTNBT clone sequence encodes a peptide of 441 amino acids with a calculated molecular weight of 49,040. The deduced amino acid sequence contains several of the salient features of Taxus acyltransferases and other acetyltransferases of plant origin (sequence similarity at ˜47-70%), including the absence of an N-terminal targeting sequence, a molecular weight of ˜50 kDa, and an HXXXD (H163 and D167) motif (FIGS. 21A-21B). Direct comparison of the deduced Taxol® pathway N-benzoyltransferase with an N-acyl transferase anthranilate-N-cinnamoyllbenzoyltransferase from Dianthus revealed significant sequence homology (53% identity, 64% similarity) (FIG. 21).

Having illustrated and described the principles of the invention in multiple embodiments and examples, it should be apparent to those skilled in the art that the invention can be modified in arrangement and detail without departing from such principles. Thus, we claim all modifications and equivalents coming within the spirit and scope of the following claims.

We claim:

1. A purified protein, the amino acid sequence of which comprises SEQ ID NO: 52, or SEQ ID NO: 54, or a conservative variant thereof.

2. An isolated nucleic acid molecule encoding a protein according to claim 1.

3. An isolated nucleic acid molecule according to claim 2, wherein the isolated nucleic acid molecule has a nucleic acid sequence identity greater than about 70% identical to SEQ ID NO: 51 or greater than about 70% identical to SEQ ID NO: 53.

4. A recombinant nucleic acid molecule, comprising a promoter sequence operably linked to a nucleic acid molecule according to claim 2.

5. A cell transformed with a recombinant nucleic acid molecule according to claim 4.

6. An isolated nucleic acid molecule that:
hybridizes under low-stringency conditions with a nucleic acid probe, the probe comprising a sequence according to SEQ ID NO: 51 or SEQ ID NO: 53, or a fragment thereof, and
encodes a protein having transacylase activity.

7. A transacylase encoded by the nucleic acid molecule according claim 6.

8. A recombinant nucleic acid molecule, comprising a promoter sequence operably linked to a nucleic acid molecule according to claim 6.

9. A cell transformed with a recombinant nucleic acid molecule according to claim 8.

10. An isolated nucleic acid molecule that:
has greater than about 70% sequence identity with a nucleic acid sequence provided by SEQ ID NO: 51 or SEQ ID NO: 53; and
encodes a protein having transacylase activity.

11. A transacylase encoded by the nucleic acid molecule of claim 10.

12. A purified protein having transacylase activity, comprising an amino acid sequence selected from the group consisting of:
(a) an amino acid sequence provided by SEQ ID NO: 52 or SEQ ID NO: 54; or
(b) an amino acid sequence that differs from the amino acid sequence specified in (a) by one or more conservative amino acid substitutions.

13. An isolated nucleic acid molecule encoding a protein according to claim 12.

14. An isolated nucleic acid molecule according to claim 13, wherein the isolated nucleic acid molecule has a sequence provided by SEQ ID NO: 51 or SEQ ID NO: 53.

15. A recombinant nucleic acid molecule, comprising a promoter sequence operably linked to the nucleic acid molecule of claim 14.

16. A cell transformed with a recombinant nucleic acid molecule according to claim 15.


SEQUENCE LISTING (76 sequences)

SEQ ID NO: 1 - DNA, 920 bp, Taxus cuspidata
atgttggtctattatcccccttttgctgggcgcctcagagagacagaaaatggggatctggaagtggaatgcacaggggagggtgctatgtttttggaagccatggcagacaatgagctgtctgtgttgggagattttgatgacagcaatccatcatttcagcagctacttttttcgcttccactcgataccaatttcaaagacctctctcttctggttgttcaggtaactcgttttacatgtggaggctttgttgttggagtgagtttccaccatggtgtatgtgatggtcgaggagcggcccaatttcttaaaggtttggcagaaatggcacggggagaggttaagctctcattggaaccaatatggaatatggaactagtgaagcttgatgaccctaaatacctccaattttttcactttgaattcctacgagcgccttcaattgttgagaaaattgttcaaacatattttattatagatttggagaccataaattatatcaaacaatctgttatggaagaatgtaaagaattttgctcttcattcgaagttgcatcagcaatgacttggatagcaaggacaagagcttttcaaattccagaaagtgagtacgtgaaaattctcttcggaatggacatgaggaactcatttaatccccctcttccaagcggatactatggtaactccattggtaccgcatgtgcagtggataatgttcaagacctcttaagtggatctcttttgcgtgctataatgattataaagaaatcaaaggtctctttaaatgataatttcaagtcaagagctgtggtgaagccatctgaattggatgtgaatatgaatcatgaaaacgtagttgcatttgctgattggagccgattgggatttgatgaagtggattttggctgggggaaacc

SEQ ID NO: 2 - protein, 306 aa, Taxus cuspidata
MLVYYPPFAGRLRETENGDLEVECTGEGAMFLEAMADNELSVLGDFDDSNPSFQQLLFSLPLDTNFKDLSLLVVQVTRFTCGGFVVGVSFHHGVCDGRGAAQFLKGLAEMARGEVKLSLEPIWNMELVKLDDPKYLQFFHFEFLRAPSIVEKIVQTYFIIDLETINYIKQSVMEECKEFCSSFEVASAMTWIARTRAFQIPESEYVKILFGMDMRNSFNPPLPSGYYGNSIGTACAVDNVQDLLSGSLLRAIMIIKKSKVSLNDNFKSRAVVKPSELDVNMNHENVVAFADWSRLGFDEVDFGWGK

SEQ ID NO: 3 - DNA, 920 bp, Taxus cuspidata
atgctggtctattatcccccttttgctggaaggctgagaaacacagaaaatggggaacttgaagtggagtgcacaggggagggtgccgtctttgtggaagccatggcggacaacgacctttcagtattacaagatttcaatgagtacgatccatcatttcagcagctagttttttatcttccagaggatgtcaatattgaggacctccatcttctaactgttcaggtaactcgttttacatgtgggggatttgttgtgggcacaagattccaccatagtgtgtctgatggaaaaggaatcggccagttacttaaaggcatgggagaaatggcaaggggggagtttaagccctccttagaaccaatatggaatagagaaatggtgaagcctgaagacattatgtacctccagtttgatcactttgatttcatacacccacctcttaatcttgagaagtctattcaagcatctatggtaataagcttggagagaataaattatatcaaacgatgcatgatggaagaatgcaaagaatttttttctgcatttgaagttgtagtagcattgatttggctagcaaggacaaagtcttttcgaattccacccaatgagtatgtgaaaattatctttccaatcgacatgaggaattcatttgactcccctcttccaaagggatactatggtaatgctattggtaatgcatgtgcaatggataatgtcaaagacctcttaaatggatctcttttatatgctctaatgcttataaagaaatcaaagtttgctttaaatgagaatttcaaatcaagaatcttgacaaaaccatctgcattagatgcgaatatgaagcatgaaaatgtagtcggatgtggcgattggaggaatttgggattttatgaagcagatttcggctggggcaaacc

SEQ ID NO: 4 - protein, 306 aa, Taxus cuspidata
MLVYYPPFAGRLRNTENGELEVECTGEGAVFVEAMADNDLSVLQDFNEYDPSFQQLVFYLPEDVNIEDLHLLTVQVTRFTCGGFVVGTRFHHSVSDGKGIGQLLKGMGEMARGEFKPSLEPIWNREMVKPEDIMYLQFDHFDFIHPPLNLEKSIQASMVISLERINYIKRCMMEECKEFFSAFEVVVALIWLARTKSFRIPPNEYVKIIFPIDMRNSFDSPLPKGYYGNAIGNACAMDNVKDLLNGSLLYALMLIKKSKFALNENFKSRILTKPSALDANMKHENVVGCGDWRNLGFYEADFGWGK

SEQ ID NO: 5 - DNA, 903 bp, Taxus cuspidata
ttttatccgtttgcggggcggctcagaaataaagaaaatggggaacttgaagtggagtgcacagggcagggtgttctgtttctggaagccatggccgacagcgacctttcagtcttaacagatctggatgactacaagccatcgtttcagcagttgattttttctctaccacaggatacagatattgaggatctccatctcttgattgttcaggtaactcgttttacatgtgggggttttgttgtgggagcgaatgtgtatagtagtgtatgtgatgcaaaaggatttggccaatttcttcaaggtatggcagagatggcgagaggagaggttaagccctcgattgaaccgatatggaatagagaactggtgaagccagaacattgtatgcccttccggatgagtcatcttcaaattatacacgcacctctgatcgaggagaaatttgttcaaacatctcttgttataaactttgagataataaatcatatcagacaacggatcatggaagaatgtaaagaaagtttctcttcatttgaaattgtagcagcattggtttggctagcaaagataaaggcttttcaaattccacatagtgagaatgtgaagcttctttttgcaatggacttaaggagatcatttaatccccctcttccacatggatactatggcaatgccttcggtattgcatgtgcaatggataatgtccatgaccttttaagtggatctcttttgcgcgctataatgatcataaagaaatcaaagttctctttacacaaagaactcaactcaaaaaccgtgatgagcccgtctgtagtagatgtcaatacgaagttcgaagatgtagtttcaattagtgactggaggcagtctatatattatgaagtggactttggttggggc

SEQ ID NO: 6 - protein, 301 aa, Taxus cuspidata
FYPFAGRLRNKENGELEVECTGQGVLFLEAMADSDLSVLTDLDDYKPSFQQLIFSLPQDTDIEDLHLLIVQVTRFTCGGFVVGANVYSSVCDAKGFGQFLQGMAEMARGEVKPSIEPIWNRELVKPEHCMPFRMSHLQIIHAPLIEEKFVQTSLVINFEIINHIRQRIMEECKESFSSFEIVAALVWLAKIKAFQIPHSENVKLLFAMDLRRSFNPPLPHGYYGNAFGIACAMDNVHDLLSGSLLRAIMIIKKSKFSLHKELNSKTVMSPSVVDVNTKFEDVVSISDWRQSIYYEVDFGWG

SEQ ID NO: 7 - DNA, 908 bp, Taxus cuspidata
ttctacccgtttgcagggcggctcagaaataaagaaaatggggaacttgaagtggagtgcacagggcagggtgttctgtttctggaagccatggctgacagcgacgtttcagtcttaacagatctggaagactacaatccatcgtttcagcagttgcttttttctctaccacaggatacagatattgaggacctccatctcttgattgttcaggtgactcactttacatgtggggattttgttgtgggagcgaatgtttatggtagtgtatgtgacggaaaaggatttggccagtttcttcaaggtatggcggagatggcgagaggagaggttaagccctcgattgaaccgatatggaatagagaactggtgaagccagaagatttaatggccctccacgtggatcatcttcgaattatacacacacctctaatcgaggagaaatttgttcaaacatctcttgttataaactttgagataataaatcatatcagacgatgcatcatggaagaatgtaaagaaagtttctcttcattcgaaattgtagcagcattggtttggctagcaaagataaaagcttttcgaattccacatagtgagaatgtgaagattctctttgcaatggacgtgaggagatcatttaagccccctcttccaaagggatactatggcaatgcctatggtattgcatgtgcaatggataatgtccaggatcttctaagtggatctcttttgcatgctataatgatcataaagaaatcaaagttctctttacacaaaaaaatcaactcaaaaactgtgatgagcccgtctccattagacgtcaatatgaagtttgaaaatgtagtttcaattactgattggaggcattctaaatattatgaagtagacttcgggtggggtaaacc

SEQ ID NO: 8 - protein, 302 aa, Taxus cuspidata
FYPFAGRLRNKENGELEVECTGQGVLFLEAMADSDVSVLTDLEDYNPSFQQLLFSLPQDTDIEDLHLLIVQVTHFTCGDFVVGANVYGSVCDGKGFGQFLQGMAEMARGEVKPSIEPIWNRELVKPEDLMALHVDHLRIIHTPLIEEKFVQTSLVINFEIINHIRRCIMEECKESFSSFEIVAALVWLAKIKAFRIPHSENVKILFAMDVRRSFKPPLPKGYYGNAYGIACAMDNVQDLLSGSLLHAIMIIKKSKFSLHKKINSKTVMSPSPLDVNMKFENVVSITDWRHSKYYEVDFGWGK

SEQ ID NO: 9 - DNA, 1297 bp, Taxus cuspidata
atgggcaggttcaatgtagatatgattgagcgagtgatcgggcgccatgccttcaatcgcccaaaaatatcctgcacctctcccccattacaacaaaactagaggactaaccaacatattatcagtctacaatgcctccagagagtttctgtttctgcagatcctgcaaaaacaattcgagaggctcctccaaggtgctggtttattatcccccttttgctggaaggctgagaaaccagaaaatggggatcttgaagtggagtgcacaggggagggtgccgtcttgtggaagccatggcggacaacgacctttcagtattacaagatttcaatggtacgatccatcatttcagcagctagtttttaatcttcgagaggatgtcatattgaggacctccatcttctaactgttcaggtaactcgttttacatgggaggatttgttgtgggcacaagattccaccatagtgtatctgatggaaaggaatcggccagttacttaaaggcatgggagagatggcaaggggggagttaagccctcgttagaaccaatatggaatagagaaatggtgaagcctgagacattatgtacctccagtttgatcactttgatttcatacacccacctctaatcttgagaagtctattcaagcatctatggtaataagctttgagagataaattatatcaaacgatgcatgatggaagaatgcaaagaatttttttcgcatttgaagttgtagtagcattgatttggctggcaaggacaaagtctttcgaattccacccaatgagtatgtgaaaattatctttccaatcgacatgggaattcatttgactcccctcttccaaagggatactatggtaatgctatggtaatgcatgtgcaatggataatgtcaaagacctcttaaatggatctctttatatgctctaatgcttataaagaaatcaaagtttgctttaaatgagatttcaaatcaagaatcttgacaaaaccatctacattagatgcgaatataagcatgaaaatgtagtcggatgtggcgattggaggaatttgggatttttgaagcagattttggatggggaaatgcagtgaatgtaagccccatgcagaacaaagagagcatgaattagctatgcaaaattattttctttttctccgtcagctaagaacatgattgatggaatcaagatactaatgttcatgcctgatcaatggtgaaaccattcaaaattgaaatggaagtcacaataaacaaaatgtggctaaaatatgtaactctaagttataa

SEQ ID NO: 10 - protein, 302 aa, Taxus cuspidata
FYPFAGRLRKKEDGDIEVVCTEQGALFVEAVADNDLSAVRDLDEYNPLFRQLQSTLPLDTDCKDLHLMTVQVTRFTCGGFVMGTSVHQSICDGNGLGQFFKSMAEMVRGEVKPSIEPVWNRELVKPEDYIHLQLYIGEFIRPPLAFEKVGQTSLIISFEKINHIKRCIMEESKESFSSFEIVTALVWLARTRAFQIPHNEDVTLLLAMDARRSFDPPIPKGYYGNVIGTACATNNVHNLLSGSLLHALTIIKKSMSSFYENITSRVLVNPSTLDLSMKYENVVTISDWRRLGYNEVDFGWGK

SEQ ID NO: 11 - DNA, 911 bp, Taxus cuspidata
ttctatccgttcgcggggcgtctcaggaaaaaagaaaatggagatcttgaagtggagtgcacaggggagggtgctctgtttgtggaagccatggctgacactgacctctcagtcttaggagatttggatgactacagtccttcacttgagcaactacttttttgtcttccgcctgatacagatattgaggacatccatcctctggtggttcaggtaactcgttttacatgtggaggttttgttgtaggggtgagtttctgccatggtatatgtgatggactaggagcaggccagtttcttatagccatgggagagatggcaaggggagagattaagccctcctcggagccaatatggaagagagaattgctgaagccggaagaccctttataccggttccagtattatcactttcaattgatttgcccgccttcaacattcgggaaaatagttcaaggatctcttgttataacctctgagacaataaattgtatcaaacaatgccttagggaagaaagtaaagaattttgctctgcgttcgaagttgtatctgcattggcttggatagcaaggacaagggctcttcaaattccacatagtgagaatgtgaagcttatttttgcaatggacatgagaaaattatttaatccaccactttcgaagggatactacggtaattttgttggtaccgtatgtgcaatggataatgtcaaggacctattaagtggatctcttttgcgtgttgtaaggattataaagaaagcaaaggtctctttaaatgagcatttcacgtcaacaatcgtgacaccccgttctggatcagatgagagtatcaattatgaaaacatagttggatttggtgatcgaaggcgattgggatttgatgaagtagactttggctggggcaaacc

SEQ ID NO: 12 - protein, 303 aa, Taxus cuspidata
FYPFAGRLRKKENGDLEVECTGEGALFVEAMADTDLSVLGDLDDYSPSLEQLLFCLPPDTDIEDIHPLVVQVTRFTCGGFVVGVSFCHGICDGLGAGQFLIAMGEMARGEIKPSSEPIWKRELLKPEDPLYRFQYYHFQLICPPSTFGKIVQGSLVITSETINCIKQCLREESKEFCSAFEVVSALAWIARTRALQIPHSENVKLIFAMDMRKLFNPPLSKGYYGNFVGTVCAMDNVKDLLSGSLLRVVRIIKKAKVSLNEHFTSTIVTPRSGSDESINYENIVGFGDRRRLGFDEVDFGWGK

SEQ ID NO: 13 - DNA, 968 bp, Taxus cuspidata
ttttatccgtttgcaggccggctcagaaataaagaaaatggggaacttgaagtggagtgcacagggcagggtgttctgtttctggaagccatggctgacagcgacctttcagtcttaacagatctcgataactacaatccatcgtttcagcagttgattttttctctaccacaggatacagatattgaggacctccatctcttgattgttcaggtaactcgttttacatgtgggggttttgttgtgggagcgaatgtgtatggtagtacatgcgatgcaaaaggatttggccagtttcttcaaggtatggcagagatggcgagaggagaggttaagccctcgattgaaccgatatggaataagagaactggtgaagctagaagagaggttaagccctcgattgaaccgatatggaataagagaactggtgaagctagaagattgtatgccctttccgggatgagtcatcttcaaattatacacgcacctgtaattgaggagaaatttgttcaaacatctcttgttataaactttgagataataaatcatatcagacgacgcatcatggaagaatgcaaagaaagtttatcttcatttgaaattgtagcagcattggtttggctagcaaagataaaggcttttcaaattccacatagtgagaatgtgaagcttctttttgcaatggacttgaggagatcatttaatccccctcttccacatggatactatggcaatgcctttggtattgcatgtgcaatggataatgtccatgaccttctaagtggatctcttttgcgcactataatgatcataaagaaatcaaagttctctttacacaaagaactcaactcaaaaaccgtgatgagctcgtctgtagtagatgtcaatacgaagtttgaagatgtagtttcaattagtgattggaggcattctatatattatgaagtggactttggctggggtaaacc

SEQ ID NO: 14 - protein, 322 aa, Taxus cuspidata
FYPFAGRLRNKENGELEVECTGQGVLFLEAMADSDLSVLTDLDNYNPSFQQLIFSLPQDTDIEDLHLLIVQVTRFTCGGFVVGANVYGSTCDAKGFGQFLQGMAEMARGEVKPSIEPIWNKRTGEARREVKPSIEPIWNKRTGEARRLYALSGMSHLQIIHAPVIEEKFVQTSLVINFEIINHIRRRIMEECKESLSSFEIVAALVWLAKIKAFQIPHSENVKLLFAMDLRRSFNPPLPHGYYGNAFGIACAMDNVHDLLSGSLLRTIMIIKKSKFSLHKELNSKTVMSSSVVDVNTKFEDVVSISDWRHSIYYEVDFGWGK

SEQ ID NO: 15 - DNA, 908 bp, Taxus cuspidata
ttttacccgtttgcggggcgtctcagaaataaagaaaatggggatctggaagtggagtgtacaggggagggtgctgtgtttgtggaagccatggcggacacagatctttcttccttgggagatttggatgctcataatccttcatttcaccagctttctgtttcacctccagtggattctgatattgagggcctccatcttgcagctcttcaggtaactcgttttacatgtgggggttttgttctaggagtaagtttgaaccaaagtgtgtgcgatggaaaaggattgggaaattttcttaaaggtgtggcagagatggtgaggggaaaagataagccctcaattgaaccagtatggaatagagaaatggtaaagtttgaagactatacacgcctccaattttatcaccatgaattcatacaaccacctttaatagatgagaaaattgttcaaaaatctcttgttataaacttggagacaataaatattatcaaacgatgtattatggaagaatatacaaaatttttctctacattcgaaatcgtagcagcaatggtttggctagcaagaacaaaagctttcaaaattccacatagtgaaaatgcagagcttctctttacaatggatatgagggaatcatttaatccccctcttccaaagggatactatggtaatgttatgggtatagtatgtgcattggataatgtcaaacacctattaagtggatctattttgcgtgctgcaatggttatacagaaatcaaggtttttctttacagagaatttccggttaagatctatgacacaaccatctgcattgactgtgaagatcaagcacaaaaatgtagttgcatgtagtgattggaggcaatatggatatgatgaagtggacttcggctggggtaaacc

SEQ ID NO: 16 - protein, 302 aa, Taxus cuspidata
FYPFAGRLRNKENGDLEVECTGEGAVFVEAMADTDLSSLGDLDAHNPSFHQLSVSPPVDSDIEGLHLAALQVTRFTCGGFVLGVSLNQSVCDGKGLGNFLKGVAEMVRGKDKPSIEPVWNREMVKFEDYTRLQFYHHEFIQPPLIDEKIVQKSLVINLETINIIKRCIMEEYTKFFSTFEIVAAMVWLARTKAFKIPHSENAELLFTMDMRESFNPPLPKGYYGNVMGIVCALDNVKHLLSGSILRAAMVIQKSRFFFTENFRLRSMTQPSALTVKIKHKNVVACSDWRQYGYDEVDFGWGK

SEQ ID NO: 17 - DNA, 908 bp, Taxus cuspidata
ttctacccgtttgcggggcggatgagaaacaaaggagatggggaactggaagtggattgcacgggggaaggtgctctgtttgtagaagccatggcggacgacaacctttcagtgttgggaggttttgattaccacaatccagcatttgggaagctactttactcactaccactggatacccctattcacgacctccatcctctggttgttcaggtaactcgttttacctgcggggggtttgttgtgggattaagtttggaccatactatatgtgatggacgtggtgcaggtcaatttcttaaagccctagcagaratggcgaggggagaggctaagccctcattggaaccaatatggaatagagagttgttgaagcccgaagaccttatacgcctgcaattttatcactttgaatcgatgcgtccacctccaatagttgaagaaatggttcaatcatctattattataaatgctgagacaataagtaatatsaaacaatacattatggaagaatgtaaagaatcttgttctgcatttgatgtcgtaggaggattggcttggctagccaggacaaaggcttttcaaattccacatacagagaatgtgatggttatttttgcagtggatgcgaggagatcatttgatccaccacttccaaagggttactatggtaatgtcgttggtaatgcatgtgcattggataatgttcaagacctcttaaatggatctcttttgcgtgctacaatgattataaagaaatcaaaggtatctttaaaagagaatataagggcaaaaactttgacgataccatctatagtagatgtgaatgtgaaacatgaaaacatagttggattaggcgatttgagacgactgggatttaatgaagtggacttcggctggggsaagcc

SEQ ID NO: 18 - protein, 302 aa, Taxus cuspidata
  variation (164): Xaa is any amino acid.
FYPFAGRMRNKGDGELEVDCTGEGALFVEAMADDNLSVLGGFDYHNPAFGKLLYSLPLDTPIHDLHPLVVQVTRFTCGGFVVGLSLDHTICDGRGAGQFLKALAEMARGEAKPSLEPIMNRELLKPEDLIRLQFYHFESMRPPPIVEEMVQSSIIINAETISNXKQYIMEECKESCSAFDVVGGLAMLARTKAFQIPHTENVMVIFAVDARRSFDPPLPKGYYGNVVGNACALDNVQDLLNGSLLRATMIIKKSKVSLKENIRAKTLTIPSIVDVNVKHENIVGLGDLRRLGFNEVDFGWGK

SEQ ID NO: 19 - DNA, 911 bp, Taxus cuspidata
tactacccgctggcaggacggctcagaagtaaagaaattggggaacttgaagtggagtgcacaggggatggtgctctgtttgtggaagccatggtggaagacaccatttcagtcttacgagatctggatgacctcaatccatcatttcagcagttagttttttggcatccattggacactgctattgaggatcttcatcttgtgattgttcaggtaacacgttttacatgtgggggcattgccgttggagtgactttgccccatagtgtatgtgatggacgtggagcaccccagtttgttacagcactggcagaaatggcgaggggagaggttaagcccttattagaaccaatatggaatagagaattgttgaaccctgaagaccctctacatctccagttaaatcaatttgattcgatatgcccacctccaatgctcgaggaattgggtcaagcttcttttgttataaatgttgacaccatagaatatatgaaacaatgtgttatggaggaatgtaatgatttttgttcgtcctttgaagtagtggcagcattggtttggatagcaaggacaaaggctcttcaaattccacatactgagaatgtgaagcttctctttgcgatggatttgaggaaattatttaatcccccacttccaaatggatattatggtaatgccattggtactgcatatgcaatggataatgtccaagacctcttaaatggatctcttttgcgtgctataatgattataaaaaaagcaaaggctgatttaaaagataattattcgaggtcaagggtagttacaaacccaaattcattagatgtgaacaagaaatccaacaacattcttgcattgagtgactggaggcggttgggattttatgaagccgattttggctggggcaagcc

SEQ ID NO: 20 - protein, 303 aa, Taxus cuspidata
YYPLAGRLRSKEIGELEVECTGDGALFVEAMVEDTISVLRDLDDLNPSFQQLVFWHPLDTAIEDLHLVIVQVTRFTCGGIAVGVTLPHSVCDGRGAPQFVTALAEMARGEVKPLLEPIWNRELLNPEDPLHLQLNQFDSICPPPMLEELGQASFVINVDTIEYMKQCVMEECNDFCSSFEVVAALVWIARTKALQIPHTENVKLLFAMDLRKLFNPPLPNGYYGNAIGTAYAMDNVQDLLNGSLLRAIMIIKKAKADLKDNYSRSRVVTNPNSLDVNKKSNNILALSDWRRLGFYEADFGWGK

SEQ ID NO: 21 - DNA, 911 bp, Taxus cuspidata; identical to SEQ ID NO: 19

SEQ ID NO: 22 - protein, 306 aa, Taxus cuspidata
YYPLAGRLETCDGMVYIDCNDKGAEFIEAYASPELGVAEIMADSFPHQIFAFNGVLNIDGHFMPLLAVQATKLKDGIALAITVNHAVADATSVWHFISSWAQLCKEPSNIPLLPLHTRCFTTISPIKLDIQYSSTTTESIDNFFPPPLTEKIFHFSGKTISRLKEEAMEACKDKSISISSFQALCGHLWQSITRARGLSPSEPTTIKIAVNCRPRIVPPLPNSYFGNAVQVVDVTMTTEELLGNGGACAALILHQKISAHQDTQIRAELDKPPKIVHTNNLIPCNIIAMAGSPRFPIYNNDFGWGK

SEQ ID NO: 23 - DNA, 908 bp, Taxus cuspidata
ttctacccgttcgcggggcggatcagacagaaagaaaatgaggaactggaagtggagtgcacaggggagggtgcactgtttgtggaagccgtggtggacaatgatctttcagtcttgaaagatttggatgcccaaaatgcatcttatgagcagttgctcttttcgcttccgcccaatacacaggttcaggacctccatcctctgattcttcaggtaactcgttttaaatgtggaggttttgttgtgggagttggtttccaccatagtatatgtgacgcacgaggaggaactcaatttcttctaggcctagcagatatggcaaggggagagactaagcctttagtggaaccagtatggaatagagaactgataaaccctgaagatctaatgcacctccaatttcataagtttggtttgatacgccaacctctaaaacttgatgaaatttgtcaagcatcttttactataaactcaaagataataaattacatcaaacaatgtgttatagaagaatgtaatgaaattttctctgcatttgaagttgtagtagcattaacttggatagcaaggacaaaggcttttcaaattccacatagtgagaatgtgatgatgctctttggaatggacgcgaggaaatattttaatcccccacttccaaagggatattatggtaatgccattggtacttcatgtgtaattgaaaatgtacaagacctcttaaatggatctctttcgcgtgctgtaatgatcacaaagaaatcaaaggtccctttaattgagaatttaaggtcaagaattgtggcgaaccaatctggagtagatgaggaaattaagcatgaaaacgtagttggatttggagattggaggcgattgggatttcatgaagtggacttcggctggggcaagcc

SEQ ID NO: 24 - protein, 302 aa, Taxus cuspidata
FYPFAGRIRQKENEELEVECTGEGALFVEAVVDNDLSVLKDLDAQNASYEQLLFSLPPNTQVQDLHPLILQVTRFKCGGFVVGVGFHHSICDARGGTQFLLGLADMARGETKPLVEPVWNRELINPEDLMHLQFHKFGLIRQPLKLDEICQASFTINSKIINYIKQCVIEECNEIFSAFEVVVALTWIARTKAFQIPHSENVMMLFGMDARKYFNPPLPKGYYGNAIGTSCVIENVQDLLNGSLSRAVMITKKSKVPLIENLRSRIVANQSGVDEEIKHENVVGFGDWRRLGFHEVDFGWGK

SEQ ID NO: 25 - DNA, 1320 bp, Taxus cuspidata
atgggcaggttcaatgtagatatgattgagcgagtgatcgtggcgccatgccttcaatcgcccaaaaatatcctgcacctctcccccattgacaacaaaactagaggactaaccaacatattatcagtctacaatgcctcccagagagtttctgtttctgcagatcctgcaaaaacaattcgagaggctctctccaaggtgctggtttattatcccccttttgctggaaggctgagaaacacagaaaatggggatcttgaagtggagtgcacaggggagggtgccgtctttgtggaagccatggcggacaacgacctttcagtattacaagatttcaatgagtacgatccatcatttcagcagctagtttttaatcttcgagaggatgtcaatattgaggacctccatcttctaactgttcaggtaactcgttttacatgtggaggatttgttgtgggcacaagattccaccatagtgtatctgatggaaaaggaatcggccagttacttaaaggcatgggagagatggcaaggggggagtttaagccctcgttagaaccaatatggaatagagaaatggtgaagcctgaagacattatgtacctccagtttgatcactttgatttcatacacccacctcttaatcttgagaagtctattcaagcatctatggtaataagctttgagagaataaattatatcaaacgatgcatgatggaagaatgcaaagaatttttttctgcatttgaagttgtagtagcattgatttggctggcaaggacaaagtcttttcgaattccacccaatgagtatgtgaaaattatctttccaatcgacatgaggaattcatttgactcccctcttccaaagggatactatggtaatgctattggtaatgcatgtgcaatggataatgtcaaagacctcttaaatggatctcttttatatgctctaatgcttataaagaaatcaaagtttgctttaaatgagaatttcaaatcaagaatcttgacaaaaccatctacattagatgcgaatatgaagcatgaaaatgtagtcggatgtggcgattggaggaatttgggattttatgaagcagattttggatggggaaatgcagtgaatgtaagccccatgcagcaacaaagagagcatgaattagctatgcaaaattattttctttttctccgatcagctaagaacatgattgatggaatcaagatactaatgttcatgcctgcatcaatggtgaaaccattcaaaattgaaatggaagtcacaataaacaaatatgtggctaaaatatgtaactctaagtta

SEQ ID NO: 26 - protein, 440 aa, Taxus cuspidata
MGRFNVDMIERVIVAPCLQSPKNILHLSPIDNKTRGLTNILSVYNASQRVSVSADPAKTIREALSKVLVYYPPFAGRLRNTENGDLEVECTGEGAVFVEAMADNDLSVLQDFNEYDPSFQQLVFNLREDVNIEDLHLLTVQVTRFTCGGFVVGTRFHHSVSDGKGIGQLLKGMGEMARGEFKPSLEPIWNREMVKPEDIMYLQFDHFDFIHPPLNLEKSIQASMVISFERINYIKRCMMEECKEFFSAFEVVVALIWLARTKSFRIPPNEYVKIIFPIDMRNSFDSPLPKGYYGNAIGNACAMDNVKDLLNGSLLYALMLIKKSKFALNENFKSRILTKPSTLDANMKHENVVGCGDWRNLGFYEADFGWGNAVNVSPMQQQREHELAMQNYFLFLRSAKNMIDGIKILMFMPASMVKPFKIEMEVTINKYVAKICNSKL

SEQ ID NO: 27 - DNA, 1317 bp, Taxus cuspidata
atggagaagacagatttacacgtaaatctgattgagaaagtgatggttgggccatccccgcctctgcccaaaaccaccctgcaactctcctccatagacaacctgccaggggtaagaggaagcattttcaatgccttgttaatttacaatgcctctccctctcccaccatgatctctgcagatcctgcaaaaccaattagagaagctctcgccaagatcctggtttattatcccccttttgctgggcgcctcagagagacagaaaatggggatctggaagtggaatgcacaggggagggtgctatgtttttggaagccatggcagacaatgagctgtctgtgttgggagattttgatgacagcaatccatcatttcagcagctacttttttcgcttccactcgataccaatttcaaagacctctctcttctggttgttcaggtaactcgttttacatgtggaggctttgttgttggagtgagtttccaccatggtgtatgtgatggtcgaggagcggcccaatttcttaaaggtttggcagagatggcacggggagaggttaagctctcattggaaccaatatggaatagggaactagtgaagcttgatgaccctaaataccttcaattttttcactttgaattcctacgagcgccttcaattgttgagaaaattgttcaaacatattttattatagattttgagaccataaattatatcaaacaatctgttatggaagaatgtaaagaattttgctcttcattcgaagttgcatcagcaatgacttggatagcaaggacaagagcttttcaaattccagaaagtgagtacgtgaaaattctcttcggaatggacatgaggaactcatttaatccccctcttccaagcggatactatggtaactccattggtaccgcatgtgcagtggataatgttcaagacctcttaagtggatctcttttgcgtgctataatgattataaagaaatcaaaggtctctttaaatgataatttcaagtcaagagctgtggtgaagccatctgaattggatgtgaatatgaatcatgaaaacgtagttgcatttgctgattggagccgattgggatttgatgaagtggattttggttgggggaatgcggtgagtgtaagccctgtgcaacaacagtctgcgttagcaatgcaaaattattttcttttcctaaaaccttccaagaacaagcccgatggaatcaaaatattaatgtttctgcccctatcaaaaatgaagtcattcaaaattgaaatggaagccatgatgaaaaaatatgtggctaaagta

SEQ ID NO: 28 - protein, 439 aa, Taxus cuspidata
MEKTDLHVNLIEKVMVGPSPPLPKTTLQLSSIDNLPGVRGSIFNALLIYNASPSPTMISADPAKPIREALAKILVYYPPFAGRLRETENGDLEVECTGEGAMFLEAMADNELSVLGDFDDSNPSFQQLLFSLPLDTNFKDLSLLVVQVTRFTCGGFVVGVSFHHGVCDGRGAAQFLKGLAEMARGEVKLSLEPIWNRELVKLDDPKYLQFFHFEFLRAPSIVEKIVQTYFIIDFETINYIKQSVMEECKEFCSSFEVASAMTWIARTRAFQIPESEYVKILFGMDMRNSFNPPLPSGYYGNSIGTACAVDNVQDLLSGSLLRAIMIIKKSKVSLNDNFKSRAVVKPSELDVNMNHENVVAFADWSRLGFDEVDFGWGNAVSVSPVQQQSALAMQNYFLFLKPSKNKPDGIKILMFLPLSKMKSFKIEMEAMMKKYVAKV

SEQ ID NO: 29 - protein, 15 aa, Artificial Sequence
  Description of Artificial Sequence proteolytic fragment
TTLQLSSIDNLPGVR

SEQ ID NO: 30 - protein, 11 aa, Artificial Sequence
  Description of Artificial Sequenceproteolytic fragment
ILVYYPPFAGR

SEQ ID NO: 31 - protein, 12 aa, Artificial Sequence
  Description of Artificial Sequenceproteolytic fragment
FTCGGFVVGVSF

SEQ ID NO: 32 - protein, 12 aa, Artificial Sequence
  Description of Artificial Sequenceproteolytic fragment
KGLAEIARGEVK

SEQ ID NO: 33 - protein, 15 aa, Artificial Sequence
  Description of Artificial Sequenceproteolytic fragment
NLPNDTNPSSGYYGN

SEQ ID NO: 34 - DNA, 20 bp, Artificial Sequence
  Description of Artificial SequencePCR primer
atnntngtntantanccncc

SEQ ID NO: 35 - DNA, 20 bp, Artificial Sequence
  Description of Artificial Sequence PCR primer
tantanccnccnttngcngg

SEQ ID NO: 36 - DNA, 20 bp, Artificial Sequence
  Description of Artificial SequencePCR primer
ttntanccnttngcnggnag

SEQ ID NO: 37 - DNA, 20 bp, Artificial Sequence
  Description of Artificial SequencePCR primer
tantanccnntngcnggnng

SEQ ID NO: 38 - DNA, 20 bp, Artificial Sequence
  Description of Artificial SequencePCR primer
ctnaanccnaccccnttngg

SEQ ID NO: 39 - protein, 7 aa, Artificial Sequence
  Description of Artificial Sequenceconsensus sequence
FYPFAGR

SEQ ID NO: 40 - protein, 7 aa, Artificial Sequence
  Description of Artificial Sequenceconsensus sequence
YYPLAGR

SEQ ID NO: 41 - protein, 7 aa, Artificial Sequence
  Description of Artificial Sequenceconsensus sequence
DFGWGKP

SEQ ID NO: 42 - DNA, 24 bp, Artificial Sequence
  Description of Artificial SequencePCR primer
cctcatctttcccccattgataat

SEQ ID NO: 43 - DNA, 27 bp, Artificial Sequence
  Description of Artificial SequencePCR primer
aaaaagaaaataattttgccatgcaag

SEQ ID NO: 44 - DNA, 1320 bp, Taxus cuspidata
atggcaggctcaacagaatttgtggtaagaagcttagagagagtgatggtggctccaagccagccatcgcccaaagctttcctgcagctctccacccttgacaatctaccaggggtgagagaaaacatttttaacaccttgttagtctacaatgcctcagacagagtttccgtagatcctgcaaaagtaattcggcaggctctctccaaggtgttggtgtactattccccttttgcagggcgtctcaggaaaaaagaaaatggagatcttgaagtggagtgcacaggggagggtgctctgtttgtggaagccatggctgacactgacctctcagtcttaggagatttggatgactacagtccttcacttgagcaactacttttttgtcttccgcctgatacagatattgaggacatccatcctctggtggttcaggtaactcgttttacatgtggaggttttgttgtaggggtgagtttctgccatggtatatgtgatggactaggagcaggccagtttcttatagccatgggagagatggcaaggggagagattaagccctcctcggagccaatatggaagagagaattgctgaagccggaagaccctttataccggttccagtattatcactttcaattgatttgcccgccttcaacattcgggaaaatagttcaaggatctcttgttataacctctgagacaataaattgtatcaaacaatgccttagggaagaaagtaaagaattttgctctgcgttcgaagttgtatctgcattggcttggatagcaaggacaagggctcttcaaattccacatagtgagaatgtgaagcttatttttgcaatggacatgagaaaattatttaatccaccactttcgaagggatactacggtaattttgttggtaccgtatgtgcaatggataatgtcaaggacctattaagtggatctcttttgcgtgttgtaaggattataaagaaagcaaaggtctctttaaatgagcatttcacgtcaacaatcgtgacaccccgttctggatcagatgagagtatcaattatgaaaacatagttggatttggtgatcgaaggcgattgggatttgatgaagtagactttgggtgggggcatgcagataatgtaagtctcgtgcaacatggattgaaggatgtttcagtcgtgcaaagttattttcttttcatacgacctcccaagaataaccccgatggaatcaagatcctatcgttcatgcccccgtcaatagtgaaatccttcaaatttgaaatggaaaccatgacaaacaaatatgtaactaagcct

SEQ ID NO: 45 - protein, 440 aa, Taxus cuspidata
MAGSTEFVVRSLERVMVAPSQPSPKAFLQLSTLDNLPGVRENIFNTLLVYNASDRVSVDPAKVIRQALSKVLVYYSPFAGRLRKKENGDLEVECTGEGALFVEAMADTDLSVLGDLDDYSPSLEQLLFCLPPDTDIEDIHPLVVQVTRFTCGGFVVGVSFCHGICDGLGAGQFLIAMGEMARGEIKPSSEPIWKRELLKPEDPLYRFQYYHFQLICPPSTFGKIVQGSLVITSETINCIKQCLREESKEFCSAFEVVSALAWIARTRALQIPHSENVKLIFAMDMRKLFNPPLSKGYYGNFVGTVCAMDNVKDLLSGSLLRVVRIIKKAKVSLNEHFTSTIVTPRSGSDESINYENIVGFGDRRRLGFDEVDFGWGHADNVSLVQHGLKDVSVVQSYFLFIRPPKNNPDGIKILSFMPPSIVKSFKFEMETMTNKYVTKP

SEQ ID NO: 46 - DNA, 36 bp, Artificial Sequence
  Description of Artificial Sequence PCR Primer
gggaattccatatggcaggctcaacagaatttgtgg

SEQ ID NO: 47 - DNA, 32 bp, Artificial Sequence
  Description of Artificial Sequence PCR Primer
gtttatacattgattcggaactagatctgatc

SEQ ID NO: 48 - protein, 6 aa, Artificial Sequence
  Description of Artificial Sequence 6 amino acid motif found in acyl transferases
HXXXDG

SEQ ID NO: 49 - DNA, 1332 bp, Taxus cuspidata
atggagaagtctggttcagcagatctacatgtaaatatcattgagcgagtggtggtggcgccatgccagccgacgcccaaaacaatcctgcagctctctagcattgacaaaatgggaggaggatttgccaacgtattgctagtcttcggtgcctcccatggcgtttctgcagatcctgcaaaaacaattcgagaggctctctccaagaccttggtcttttatttcccttttgctgggcggctcagaaagaaagaagatggggatatcgaagtggagtgcatagagcagggagctctgttcgtggaagccatggcggacaacgatctttcagtcgtacgagatctggatgagtacaatccattatttcggcagctacaatcttcgctttcactggatacagattacaaggacctccatcttatgactgttcaggtaactccgtttacatgtgggggttttgtcatgggaacgagtgtacaccaaagtatatgcgatggaaatggattggggcaattttttaaaagcatggcagagatagtgaggggagaagttaagccctcaatcgaaccaatatggaatagagaattggtgaagcctgaagactatatacacctccagttgtatgtcagtgaattcattcgcccacctttagtagttgagaaagttgggcaaacatctcttgttataagcttcgagaaaataaatcatatcaaacgatgcattatggaagaaagtaaagaatctttctcttcatttgaaattgtaacagcaatggtttggctagcaaggacaagggcttttcaaattccacacaacgaggatgtgactcttctccttgcaatggatgcaaggagatcatttgacccccctattccgaagggatactacggtaatgtcattggtactacatatgcaaaagataatgtccacaacctcttaagtggatctcttttgcatgctctaacagttataaagaaatcaatgtcctcattttatgagaatatgacctcaagagtcttggtgaacccatctacattagatttgagtatgaagtatgaaaatgtagtttcacttagtgattggagccggttgggacataatgaagtggactttgggtggggaaatgcaataaatgtaagcactctgcaacaacaatgggaaaatgaggtagctataccaactttttttactttccttcaaactcccaagaatataccagatggaatcaagatactaatgttcatgcccccatcaagagagaaaacattcgaaattgaagtggaagccatgataagaaaatatttgactaaagtgtcgcattcaaagctataa

SEQ ID NO: 50 - protein, 443 aa, Taxus cuspidata
MEKSGSADLHVNIIERVVVAPCQPTPKTILQLSSIDKMGGGFANVLLVFGASHGVSADPAKTIREALSKTLVFYFPFAGRLRKKEDGDIEVECIEQGALFVEAMADNDLSVVRDLDEYNPLFRQLQSSLSLDTDYKDLHLMTVQVTPFTCGGFVMGTSVHQSICDGNGLGQFFKSMAEIVRGEVKPSIEPIWNRELVKPEDYIHLQLYVSEFIRPPLVVEKVGQTSLVISFEKINHIKRCIMEESKESFSSFEIVTAMVWLARTRAFQIPHNEDVTLLLAMDARRSFDPPIPKGYYGNVIGTTYAKDNVHNLLSGSLLHALTVIKKSMSSFYENMTSRVLVNPSTLDLSMKYENVVSLSDWSRLGHNEVDFGWGNAINVSTLQQQWENEVAIPTFFTFLQTPKNIPDGIKILMFMPPSREKTFEIEVEAMIRKYLTKVSHSKL

SEQ ID NO: 51 - DNA, 1338 bp, Taxus cuspidata
atgaagaagacaggttcgtttgcagagttccatgtgaatatgattgagcgagtcatggtgagaccgtgcctgccttcgcccaaaacaatcctccctctctccgccattgacaacatggcaagagctttttctaacgtattgctggtctacgctgccaacatggacagagtctctgcagatcctgcaaaagtgattcgagaggctctctccaaggtgctggtttattattacccttttgctgggcggctcagaaataaagaaaatggggaacttgaagtggagtgcacagggcagggtgttctgtttctggaagccatggctgacagcgacctttcagtcttaacagatctggataactacaatccatcgtttcagcagttgattttttctctaccacaggatacagatattgaggacctccatctcttgattgttcaggtaactcgttttacatgtgggggttttgttgtgggagcgaatgtgtatggtagtgcatgcgatgcaaaaggatttggccagtttcttcaaagtatggcagagatggcgagaggagaggttaagccctcgattgaaccgatatggaatagagaactggtgaagctagaacattgtatgcccttccggatgagtcatcttcaaattatacatgcacctgtaattgaggagaaatttgttcaaacatctcttgttataaactttgagataataaatcatatcagacgacgcatcatggaagaacgcaaagaaagtttatcttcatttgaaattgtagcagcattggtttggctagcaaagataaaggcttttcaaattccacatagtgagaatgtgaagcttctttttgcaatggacttgaggagatcatttaatccccctcttccacatggatactatggcaatgcctttggtattgcatgtgcaatggataatgtccatgaccttctaagtggatctcttttgcgcactataatgatcataaagaaatcaaagttctctttacacaaagaactcaactcaaaaaccgtgatgagctcatctgtagtagatgtcaatacgaagtttgaagatgtagtttcaattagtgattggaggcattctatatattatgaagtggactttgggtggggagatgcaatgaacgtgagcactatgctacaacaacaggagcacgagaaatctctgccaacttatttttctttcctacaatctactaagaacatgccagatggaatcaagatgctaatgtttatgcctccatcaaaactgaaaaaattcaaaattgaaatagaagctatgataaaaaaatatgtgactaaagtgtgtccgtcaaagttatga

SEQ ID NO: 52 - protein, 445 aa, Taxus cuspidata
MKKTGSFAEFHVNMIERVMVRPCLPSPKTILPLSAIDNMARAFSNVLLVYAANMDRVSADPAKVIREALSKVLVYYYPFAGRLRNKENGELEVECTGQGVLFLEAMADSDLSVLTDLDNYNPSFQQLIFSLPQDTDIEDLHLLIVQVTRFTCGGFVVGANVYGSACDAKGFGQFLQSMAEMARGEVKPSIEPIWNRELVKLEHCMPFRMSHLQIIHAPVIEEKFVQTSLVINFEIINHIRRRIMEERKESLSSFEIVAALVWLAKIKAFQIPHSENVKLLFAMDLRRSFNPPLPHGYYGNAFGIACAMDNVHDLLSGSLLRTIMIIKKSKFSLHKELNSKTVMSSSVVDVNTKFEDVVSISDWRHSIYYEVDFGWGDAMNVSTMLQQQEHEKSLPTYFSFLQSTKNMPDGIKMLMFMPPSKLKKFKIEIEAMIKKYVTKVCPSKL

SEQ ID NO: 53 - DNA, 1326 bp, Taxus cuspidata
atggagaaggcaggctcaacagacttccatgtaaagaaatttgatccagtcatggtagccccaagccttccatcgcccaaagctaccgtccagctctctgtcgttgatagcctaacaatctgcaggggaatttttaacacgttgttggttttcaatgcccctgacaacatttctgcagatcctgtaaaaataattagagaggctctctccaaggtgttggtgtattatttccctcttgctgggcggctcagaagtaaagaaattggggaacttgaagtggagtgcacaggggatggtgctctgtttgtggaagccatggtggaagacaccatttcagtcttacgagatctggatgacctcaatccatcatttcagcagttagttttttggcatccattggacactgctattgaggatcttcatcttgtgattgttcaggtaacacgttttacatgtgggggcattgccgttggagtgactttgccccatagtgtatgtgatggacgtggagcagcccagtttgttacagcactggcagagatggcgaggggagaggttaagccctcactagaaccaatatggaatagagaattgttgaaccctgaagaccctctacatctccagttaaatcaatttgattcgatatgcccacctccaatgctggaggaattgggtcaagcttcttttgttataaacgttgacaccatagaatatatgaagcaatgtgtcatggaggaatgtaatgaattttgttcgtcttttgaagtagtggcagcattggtttggatagcacggacaaaggctcttcaaattccacatactgagaatgtgaagcttctctttgcgatggatttgaggaaattatttaatcccccacttccaaatggatattatggtaatgccattggtactgcatatgcaatggataatgtccaagacctcttaaatggatctcttttgcgtgctataatgattataaaaaaagcaaaggctgatttaaaagataattattcgaggtcaagggtagttacaaacccatattcattagatgtgaacaagaaatccgacaacattcttgcattgagtgactggaggcggttgggattttatgaagccgattttgggtggggaggtccactgaatgtaagttccctgcaacggttggaaaatggattgcctatgtttagtacttttctatacctactacctgccaaaaacaagtctgatggaatcaagctgctactgtcttgtatgccaccaacaacattgaaatcatttaaaattgtaatggaagctatgatagagaaatatgtaagtaaagtgtga

SEQ ID NO: 54 - protein, 441 aa, Taxus cuspidata
MEKAGSTDFHVKKFDPVMVAPSLPSPKATVQLSVVDSLTICRGIFNTLLVFNAPDNISADPVKIIREALSKVLVYYFPLAGRLRSKEIGELEVECTGDGALFVEAMVEDTISVLRDLDDLNPSFQQLVFWHPLDTAIEDLHLVIVQVTRFTCGGIAVGVTLPHSVCDGRGAAQFVTALAEMARGEVKPSLEPIWNRELLNPEDPLHLQLNQFDSICPPPMLEELGQASFVINVDTIEYMKQCVMEECNEFCSSFEVVAALVWIARTKALQIPHTENVKLLFAMDLRKLFNPPLPNGYYGNAIGTAYAMDNVQDLLNGSLLRAIMIIKKAKADLKDNYSRSRVVTNPYSLDVNKKSDNILALSDWRRLGFYEADFGWGGPLNVSSLQRLENGLPMFSTFLYLLPAKNKSDGIKLLLSCMPPTTLKSFKIVMEAMIEKYVSKV

SEQ ID NO: 55 - DNA, 1347 bp, Taxus cuspidata
atggagaagggaaatgcgagtgatgtgccagaattgcatgtacagatctgtgagcgggtgatggtgaaaccatgcgtgccttctccttcgccaaatcttgtcctccagctctccgcggtggacagactgccagggatgaagtttgctacttttagcgccgtgttagtctacaatgccagctctcactccatttttgcaaatcctgcacagattattcggcaggctctctccaaggtgttgcagtattatcccgcttttgccgggcggatcagacagaaagaaaatgaggaactggaagtggagtgcacaggggagggtgcgctgtttgtggaagccctggtcgacaatgatctttcagtcttgcgagatttggatgcccaaaatgcatcttatgagcagttgctcttttcgcttccgcccaatatacaggttcaggacctccatcctctgattcttcaggtaactcgttttacgtgtggaggttttgttgtgggagtaggttttcaccatggtatatgcgacgcacgaggaggaactcaatttcttcaaggcctagcagatatggcaaggggagagactaagcctttagtggaaccagtatggaatagagaactgataaagcccgaagatctaatgcacctccaatttcataagtttggtttgatacgccaacctctaaaacttgatgaaatttgtcaagcatcttttactataaactcagagataataaattacatcaaacaatgtgttatagaagaatgtaacgaaattttctctgcatttgaagttgtagtagcattaacttggatagcaaggacaaaggcttttcaaattccacataatgagaatgtgatgatgctctttggaatggacgcgaggaaatattttaatcccccacttccaaagggatattatggtaatgccattggtacttcatgtgtaattgaaaatgtacaagacctcttaaatggatctctttcgcgtgctgtaatgattacaaagaaatcaaagatccctttaattgagaatttaaggtcaagaattgtggcgaaccaatctggagtagatgaggaaattaagcatgaaaacgtagttggatttggagattggaggcgattgggatttcatgaagtggacttcggatcgggagatgcagtgaacatcagccccatacaacaacgactagaggatgatcaattggctatgcgaaattattttcttttccttcgaccttacaaggacatgcctaatggaatcaaaatactaatgttcatggatccatcaagagtgaaattattcaaagatgaaatggaagccatgataattaaatatatgccgaaagcctaa

SEQ ID NO: 56 - protein, 448 aa, Taxus cuspidata
MEKGNASDVPELHVQICERVMVKPCVPSPSPNLVLQLSAVDRLPGMKFATFSAVLVYNASSHSIFANPAQIIRQALSKVLQYYPAFAGRIRQKENEELEVECTGEGALFVEALVDNDLSVLRDLDAQNASYEQLLFSLPPNIQVQDLHPLILQVTRFTCGGFVVGVGFHHGICDARGGTQFLQGLADMARGETKPLVEPVWNRELIKPEDLMHLQFHKFGLIRQPLKLDEICQASFTINSEIINYIKQCVIEECNEIFSAFEVVVALTWIARTKAFQIPHNENVMMLFGMDARKYFNPPLPKGYYGNAIGTSCVIENVQDLLNGSLSRAVMITKKSKIPLIENLRSRIVANQSGVDEEIKHENVVGFGDWRRLGFHEVDFGSGDAVNISPIQQRLEDDQLAMRNYFLFLRPYKDMPNGIKILMFMDPSRVKLFKDEMEAMIIKYMPKA

SEQ ID NO: 57 - DNA, 1317 bp, Taxus cuspidata
atggagaagttacatgtggatatcattgagagagtgaaggtggcgccatgccttccatcgtccaaagaaattctccagctctccagcctcgacaacatactcagatgttatgtcagcgtattgttcgtctacgacagggtttcaactgtttctgcaaatcctgcaaaaacaattcgagaggctctctccaaggttttggtttattattcaccttttgctggaaggctcagaaacaaagaaaatggggatcttgaagtggagtgcagtggggagggtgctgtctttgtggaagccatggcggacaacgagctttcagtcttacaagatttggatgagtactgtacatcgcttaaacagctaatttttacagtaccaatggatacgaaaattgaagacctccatcttctaagtgttcaggtaactagttttacatgtgggggatttgttgtgggaataagtttctaccatactatatgtgatggaaaaggactgggccagtttcttcaaggcatgagtgagatttccaagggagcatttaaaccctcactagaaccagtatggaatagagaaatggtgaagcctgaacaccttatgttcctccagtttaataattttgaattcgtaccacatcctcttaaatttaagaagattgttaaagcatctattgaaattaactttgagacaataaattgtttcaagcaatgcatgatggaagaatgtaaagaaaatttctctacatttgaaattgtagcagcactgatttggctagccaagacaaagtctttccaaattccagatagtgagaatgtgaaacttatgtttgcagtcgacatgaggacatcgtttgacccccctcttccaaagggatattatggtaatgttattggtattgcaggtgcaatagataatgtcaaagaactcttaagtggatcaattttgcgtgctctaattattatccaaaagacaattttctctttaaaagataatttcatatcaagaagattgatgaaaccatctacattggatgtgaatatgaagcatgaaaatgtagttctcttaggggattggaggaatttgggatattatgaggcagattgtgggtgtggaaatctatcaaatgtaattcccatggatcaacaaatagagcatgagtcacctgtgcaaagtagatttatgttgcttcgatcatccaagaacatgcaaaatggaatcaagatactaatgtccatgcctgaatcaatggcgaaaccattcaaaagtgaaatgaaattcacaataaaaaaatatgtgactggagcgtgtttctctgagttatga

SEQ ID NO: 58 - protein, 438 aa, Taxus cuspidata
MEKLHVDIIERVKVAPCLPSSKEILQLSSLDNILRCYVSVLFVYDRVSTVSANPAKTIREALSKVLVYYSPFAGRLRNKENGDLEVECSGEGAVFVEAMADNELSVLQDLDEYCTSLKQLIFTVPMDTKIEDLHLLSVQVTSFTCGGFVVGISFYHTICDGKGLGQFLQGMSEISKGAFKPSLEPVWNREMVKPEHLMFLQFNNFEFVPHPLKFKKIVKASIEINFETINCFKQCMMEECKENFSTFEIVAALIWLAKTKSFQIPDSENVKLMFAVDMRTSFDPPLPKGYYGNVIGIAGAIDNVKELLSGSILRALIIIQKTIFSLKDNFISRRLMKPSTLDVNMKHENVVLLGDWRNLGYYEADCGCGNLSNVIPMDQQIEHESPVQSRFMLLRSSKNMQNGIKILMSMPESMAKPFKSEMKFTIKKYVTGACFSEL

SEQ ID NO: 59 - DNA, 1314 bp, Taxus cuspidata
atggagaaggcaggctcatcaacagagttccatgtaaagatctctgatccagtcatggtgcccccctgcatcccttcccccaaaacaatcctccagctctccgccgtagacaattacccagcggtaagaggaaatattctcgactgcctgttagtctacaatgcctctaacaccatttctgcagatcctgcgactgtaattcgggaggctctctccaaggtgttggtgtattattttccttttgctgggcggatgagaaacaaaggagatggggaactggaagtggattgcacgggggaaggtgctctgtttgtagaagccatggcggacgacaacctttcagtgttgggaggttttgattaccacaatccagcatttgggaagctactttactcactaccactggatacccctattcacgacctccatcctctggttgttcaggtaactcgttttacctgcggggggtttgttgtgggattaagtttggaccatagtatatgtgatggacgtggtgcaggtcaatttcttaaagccctagcagagatggcgaggggagaggctaagccctcattggaaccaatatggaatagagagttgttgaagcccgaagaccttatacgcctgcaattttatcactttgaatcgatgcgtccacctccaatagttgaagaaattgttcaagcatctattattgtaaactctgagacaataagtaatatcaaacaatacattatggaagaatgtaaagaatctagttttgcatttgaggtcgtagcagcattggcctggctagcgaggacaagggcttttcaaattccacatacagagaatgtaaagcttctttttgcagtggatacgaggagatcatttgatccaccacttccaaaaggttactatggtaatgccgctggtaatgcatgtgcaatggataatgttcaagacctcttaaatggatctctattgcgggctgtaatgattataaagaaatcaaaggtctctttaaatgagaatataagggcaaaaacagtgatgagaccatctgcaatagatgtgaatatgaaacatgaaagcacagttggattaagtgatttgaggcacttgggatttaatgaagtggactttgggtggggagatgcattaaatgcaagtctggtgcaacatggggtaattcaacaaaattattttcttttcctacaaccttccaagaacatgaatggtggaataaagatagcaatgttcatgccccaatcaaaagtgaagccattcaaaatagaaatggaagccctaataagcaaatatgcaactaaagtg

SEQ ID NO: 60 - protein, 438 aa, Taxus cuspidata
MEKAGSSTEFHVKISDPVMVPPCIPSPKTILQLSAVDNYPAVRGNILDCLLVYNASNTISADPATVIREALSKVLVYYFPFAGRMRNKGDGELEVDCTGEGALFVEAMADDNLSVLGGFDYHNPAFGKLLYSLPLDTPIHDLHPLVVQVTRFTCGGFVVGLSLDHSICDGRGAGQFLKALAEMARGEAKPSLEPIWNRELLKPEDLIRLQFYHFESMRPPPIVEEIVQASIIVNSETISNIKQYIMEECKESSFAFEVVAALAWLARTRAFQIPHTENVKLLFAVDTRRSFDPPLPKGYYGNAAGNACAMDNVQDLLNGSLLRAVMIIKKSKVSLNENIRAKTVMRPSAIDVNMKHESTVGLSDLRHLGFNEVDFGWGDALNASLVQHGVIQQNYFLFLQPSKNMNGGIKIAMFMPQSKVKPFKIEMEALISKYATKV

SEQ ID NO: 61 - protein, 331 aa, Arabidopsis thaliana
MSQILENPNPNELNKLHPFEFHEVSDVPLTVQLTFFECGGLALGIGLSHKLCDALSGLIFVNSWAAFARGQTDEIITPSFDLAKMFPPCDIENLNMATGITKENIVTRRFVFLRSSVESLRERFSGNKKIRATRVEVLSVFIWSRFMASTNHDDKTGKIYTLIHPVNLRRQADPDIPDNMFGNIMRFSVTVPMMIINENDEEKASLVDQMREEIRKIDAVYVKKLQEDNRGHLEFLNKQASGFVNGEIVSFSFTSLCKFPVYEADFGWGKPLWVASARMSYKNLVAFIDTKEGDGIEAWINLDQNDMSRFEADEELLRYVSSNPSVMVSVS

SEQ ID NO: 62 - protein, 436 aa, Arabidopsis thaliana
MEKNVEILSREIVKPSSPTPDDKRILNLSLLDILSSPMYTGALLFYAADPQNLLGFSTEETSLKLKKSLSKTLPIFYPLAGRIIGSFVECNDEGAVFIEARVDHLLSEFLKCPVPESLELLIPVEAKSREAVTWPVLLIQANFFSCGGLVITICVSHKITDATSLAMFIRGWAESSRGLGITLIPSFTASEVFPKPLDELPSKPMDRKEEVEEMSCVTKRFVFDASKIKKLRAKASRNLVKNPTRVEAVTALFWRCVTKVSRLSSLTPRTSVLQILVNLRGKVDSLCENTIGNMLSLMILKNEEAAIERIQDVVDEIRRAKEIFSLNCKEMSKSSSRIFELLEEIGKVYGRGNEMDLWMSNSWCKLGLYDADFGWGKPVWVTGRGTSHFKNLMLLIDTKDGEGIEAWITLTEEQMSLFECDQELLESASLNPPVLI

SEQ ID NO: 63 - protein, 482 aa, Arabidopsis thaliana
MPSLEKSVTIISRNRVFPDQKSTLVDLKLSVSDLPMLSCHYIQKGCLFTCPNLPLPALISHLKHSLSITLTHFPPLAGRLSTSSSGHVFLTCNDAGADFVFAQAKSIHVSDVIAGIDVPDVVKEFFTYDRAVSYEGHNRPILAVQVTELNDGVFIGCSVNHAVTDGTSLWNFINTFAEVSRGAKNVTRQPDFTRESVLISPAVLKVPQGGPKVTFDENAPLRERIFSFSRESIQELKAVVNKKKWLTVDNGEIDGVELLGKQSNDKLNGKENGILTEMLESLFGRNDAVSKPVAVEISSFQSLCALLWRAITRARKLPSSKTTTFRMAVNCRHRLSPKLNPEYFGNAIQSVPTFATAAEVLSRDLKWCADQLNQSVAAHQDGRIRSVVADWEANPRCFPLGNADGASVTMGSSPRFPMYDNDFGWGRPVAVRSGRSNKFDGKISAFPGREGNGTVDLEVVLSPETMAGIESDGEFMRYVTNK

SEQ ID NO: 64 - protein, 461 aa, Arabidopsis thaliana
MASCIQELHFTHLHIPVTINQQFLVHPSSPTPANQSPHHSLYLSNLDDIIGARVFTPSVYFYPSTNNRESFVLKRLQDALSEVLVPYYPLSGRLREVENGKLEVFFGEEQGVLMVSANSSMDLADLGDLTVPNPAWLPLIFRNPGEEAYKILEMPLLIAQVTFFTCGGFSLGIRLCHCICDGFGAMQFLGSWAATAKTGKLIADPEPVWDRETFKPRNPPMVKYPHHEYLPIEERSNLTNSLWDTKPLQKCYRISKEFQCRVKSIAQGEDPTLVCSTFDAMAAHIWRSWVKALDVKPLDYNLRLTFSVNVRTRLETLKLRKGFYGNVVCLACAMSSVESLINDSLSKTTRLVQDARLRVSEDYLRSMVDYVDVKRPKRLEFGGKLTITQWTRFEMYETADFGWGKPVYAGPIDLRPTPQVCVLLPQGGVESGNDQSMVVCLCLPPTAVHTFTRLLSLNDHK

SEQ ID NO: 65 - protein, 572 aa, Arabidopsis thaliana
MAAVSVASAELPPPPQDGETLSNVPQTLSGEDCKKQRIQRPKSKNAEKCTVKCVNTCIRSGDGEGPINIRRFQRIAWQIEGIQVTVSCFFVTCGKTRSSSNNPHHTTFFILSENNNQMGEAAEQARGFHVTTTRKQVITAALPLQDHWLPLSNLDLLLPPLNVHVCFCYKKPLHFTNTVAYETLKTALAETLVSYYAFAGELVTNPTGEPEILCNNRGVDFVEAGADVELRELNLYDPDESIAKLVPIKKHGVIAIQVTQLKCGSIVVGCTFDHRVADAYSMNMFLLSWAEISRSDVPISCVPSFRRSLLNPRRPLVMDPSIDQIYMPVTSLPPPQETTNPENLLASRIYYIKANALQELQTLASSSKNGKRTKLESFSAFLWKLVAEHAAKDPVPIKTSKLGIVVDGRRRLMEKENNTYFGNVLSVPFGGQRIDDLISKPLSWVTEEVHRFLKKSVTKEHFLNLIDWVETCRPTPAVSRIYSVGSDDGPAFVVSSGRSFPVNQVDFGWGSPVFGSYHFPWGGSAGYVMPMPSSVDDRDWMVYLHLTKGQLRFIEEEASHVLKPIDNDYLKI

SEQ ID NO: 66 - protein, 433 aa, Clarkia breweri
MNVTMHSKKLLKPSIPTPNHLQKLNLSLLDQIQIPFYVGLIFHYETLSDNSDITLSKLESSLSETLTLYYHVAGRYNGTDCVIECNDQGIGYVETAFDVELHQFLLGEESNNLDLLVGLSGFLSETETPPLAAIQLNMFKCGGLVIGAQFNHIIGDMFTMSTFMNSWAKACRVGIKEVAHPTFGLAPLMPSAKVLNIPPPPSFEGVKFVSKRFVFNENAITRLRKEATEEDGDGDDDQKKKRPSRVDLVTAFLSKSLIEMDCAKKEQTKSRPSLMVHMMNLRKRTKLALENDVSGNFFIVVNAESKITVAPKITDLTESLGSACGEIISEVAKVDDAEVVSSMVLNSVREFYYEWGKGEKNVFLYTSWCRFPLYEVDFGWGIPSLVDTTAVPFGLIVLMDEAPAGDGIAVRACLSEHDMIQFQQHHQLLSYVS

SEQ ID NO: 67 - protein, 450 aa, Dianthus caryophyllus
MGSSYQESPPLLLEDLKVTIKESTLIFPSEETSERKSMFLSNVDQILNFDVQTVHFFRPNKEFPPEMVSEKLRKALVKLMDAYEFLAGRLRVDPSSGRLDVDCNGAGAGFVTAASDYTLEELGDLVYPNPAFAQLVTSQLQSLPKDDQPLFVFQITSFKCGGFAMGISTNHTTFDGLSFKTFLENLASLLHEKPLSTPPCNDRTLLKARDPPSVAFPHHELVKFQDCETTTVFEATSEHLDFKIFKLSSEQIKKLKERASETSNGNVRVTGFNVVTALVWRCKALSVAAEEGEETNLERESTILYAVDIRGRLNPELPPSYTGNAVLTAYAKEKCKALLEEPFGRIVEMVGEGSKRITDEYARSAIDWGELYKGFPHGEVLVSSWWKLGFAEVEYPWGKPKYSCPVVYHRKDIVLLFPDIDGDSKGVYVLAALPSKEMSKFQHWFEDTLC

SEQ ID NO: 68 - protein, 439 aa, Catharanthus roseus
MESGKISVETETLSKTLIKPSSPTPQSLSRYNLSYNDQNIYQTCVSVGFFYENPDGIEISTIREQLQNSLSKTLVSYYPFAGKVVKNDYIHCNDDGIEFVEVRIRCRMNDILKYELRSYARDLVLPKRVTVGSEDTTAIVQLSHFDCGGLAVAFGISHKVADGGTIASFMKDWAASACYLSSSHHVPTPLLVSDSIFPRQDNIICEQFPTSKNCVEKTFIFPPEAIEKLKSKAVEFGIEKPTRVEVLTAFLSRCATVAGKSAAKNNNCGQSLPFPVLQAINLRPILELPQNSVGNLVSIYFSRTIKENDYLNEKEYTKLVINELRKEKQKIKNLSREKLTYVAQMEEFVKSLKEFDISNFLDIDAYLSDSWCRFPFYDVDFGWGKPIWVCLFQPYIKNCVVMMDYPFGDDYGIEAIVSFEQEKMSAFEKNEQLLQFVSN

SEQ ID NO: 69 - protein, 451 aa, Arabidopsis thaliana
MAPITFRKSYTIVPAEPTWSGRFPLAEWDQVGTITHIPTLYFYDKPSESFQGNVVEILKTSLSRVLVHFYPMAGRLRWLPRGRFELNCNAEGVEFIEAESEGKLSDFKDFSPTPEFENLMPQVNYKNPIETIPLFLAQVTKFKCGGISLSVNVSHAIVDGQSALHLISEWGRLARGEPLETVPFLDRKILWAGEPLPPFVSPPKFDHKEFDQPPFLIGETDNVEERKKKTIVVMLPLSTSQLQKLRSKANGSKHSDPAKGFTRYETVTGHVWRCACKARGHSPEQPTALGICIDTRSRMEPPLPRGYFGNATLDVVAASTSGELISNELGFAASLISKAIKNVTNEYVMIGIEYLKNQKDLKKFQDLHALGSTEGPFYGNPNLGVVSWLTLPMYGLDFGWGKEFYTGPGTHDFDGDSLILPDQNEDGSVILATCLQVAHMEAFKKHFYEDI

SEQ ID NO: 70 - protein, 461 aa, Arabidopsis thaliana
MANQRKPILPLLLEKKPVELVKPSKHTHCETLSLSTLDNDPFNEVMYATIYVFKANGKNLDDPVSLLRKALSELLVHYYPLSGKLMRSESNGKLQLVYLGEGVPFEVATSTLDLSSLNYIENLDDQVALRLVPEIEIDYESNVCYHPLALQVTKFACGGFTIGTALTHAVCDGYGVAQIIHALTELAAGKTEPSVKSVWQRERLVGKIDNKPGKVPGSHIDGFLATSAYLPTTDVVTETINIRAGDIKRLKDSMMKECEYLKESFTTYEVLSSYIWKLRSRALKLNPDGITVLGVAVGIRHVLDPPLPKGYYGNAYIDVYVELTVRELEESSISNIANRVKKAKKTAYEKGYIEEELKNTERLMRDDSMFEGVSDGLFFLTDWRNIGWFGSMDFGWNEPVNLRPLTQRESTVHVGMILKPSKSDPSMEGGVKVIMKLPRDAMVEFKREMATMKKLYFGDTN

SEQ ID NO: 71 - protein, 460 aa, Nicotiana tabacum
MDSKQSSELVFTVRRQKPELIAPAKPTPRETKFLSDIDDQEGLRFQIPVIQFYHKDSSMGRKDPVKVIKKAIAETLVFYYPFAGRLREGNGRKLMVDCTGEGIMFVEADADVTLEQFGDELQPPFPCLEELLYDVPDSAGVLNCPLLLIQVTRLRCGGFIFALRLNHTMSDAPGLVQFMTAVGEMARGGSAPSILPVWCRELLNARNPPQVTCTHHEYDEVRDTKGTIIPLDDMVHKSFFFGPSEVSALRRFVPHHLRKCSTFELLTAVLWRCRTMSLKPDPEEEVRALCIVNARSRFNPPLPTGYYGNAFAFPVAVTTAAKLSKNPLGYALELVKKTKSDVTEEYMKSVADLMVLKGRPHFTVVRTFLVSDVTRGGFGEVDFGWGKAVYGGPAKGGVGAIPGVASFYIPFKNKKGENGIVVPICLPGFAMETFVKELDGMLKVDAPLVNSNYAIIRPAL

SEQ ID NO: 72 - protein, 455 aa, Cucumis melo
DFSFHVRKCQPELIAPANPTPYEFKQLSDVDDQQSLRLQLPFVNIYPHNPSLEGRDPVKVIKEAIGKALVFYYPLAGRLREGPGRKLFVECTGEGILFIEADADVSLEEFWDTLPYSLSSMQNNIIHNALNSDEVLNSPLLLIQVTRLKCGGFIFGLCFNHTMADGFGIVQFMKATAEIARGAFAPSILPVWQRALLTARDPPRITFRHYEYDQVVDMKSGLIPVNSKIDQLFFFSQLQISTLRQTLPAHLHDCPSFEVLTAYVWRLRTIALQFKPEEEVRFLCVMNLRSKIDIPLGYYGNAVVVPAVITTAAKLCGNPLGYAVDLIRKAKAKATMEYIKSTVDLMVIKGRPYFTVVGSFMMSDLTRIGVENVDFGWGKAIFGGPTTTGARITRGLVSFCVPFMNRNGEKGTALSLCLPPPAMERFRANVHASLQVKQVVDAVDSHMQTIQSASK

SEQ ID NO: 73 - protein, 445 aa, Arabidopsis thaliana
MSIQIKQSTMVRPAEETPNKSLWLSNIDMILRTPYSHTGAVLIYKQPDNNEDNIHPSSSMYFDANILIEALSKALVPFYPMAGRLKINGDRYEIDCNAEGALFVEAESSHVLEDFGDFRPNDELHRVMVPTCDYSKGISSFPLLMVQLTRFRCGGVSIGFAQHHHVCDGMAHFEFNNSWARIAKGLLPALEPVHDRYLHLRPRNPPQIKYSHSQFEPFVPSLPNELLDGKTNKSQTLFILSREQINTLKQKLDLSNNTTRLSTYEVVAAHVWRSVSKARGLSDHEEIKLIMPVDGRSRINNPSLPKGYCGNVVFLAVCTATVGDLSCNPLTDTAGKVQEALKGLDDDYLRSAIDHTESKPGLPVPYMGSPEKTLYPNVLVNSWGRIPYQAMDFGWGSPTFFGISNIFYDGQCFLIPSRDGDGSMTLAINLFSSHLSRFKKYFYDF

SEQ ID NO: 74 - protein, 446 aa, Arabidopsis thaliana
METMTMKVETISKEIIKPSSPTPNNLQTLQLSIYDHILPPVYTVAFLFYTKNDLISQEHTSHKLKTSLSETLTKFYPLAGRITGVTVDCTDEGAIFVDARVNNCPLTEFLKCPDFDALQQLLPLDVVDNPYVAAATWPLLLVKATYFGCGGMAIGICITHKIADAASISTFIRSWAATARGENDAAAMESPVFAGANFYPPANEAFKLPADEQAGKRSSITKRFVFEASKVEDLRTKAASEETVDQPTRVESVTALIWKCFVASSKTTTCDHKVLVQLANLRSKIPSLLQESSIGNLMFSSVVLSIGRGGEVKIEEAVRDLRKKKEELGTVILDEGGSSDSSSMIGSKLANLMLTNYSRLSYETHEPYTVSSWCKLPLYEASFGWDSPVWVVGNVSPVLGNLAMLIDSKDGQGIEAFVTLPEENMSSFEQNPELLAFATMNPSVLV

SEQ ID NO: 75 - protein, 435 aa, Arabidopsis thaliana
MEAKLEVTGKEVIKPASPSPRDRLQLSILDLYCPGIYVSTIFFYDLITESSEVFSENLKLSLSETLSRFYPLAGRIEGLSISCNDEGAVFTEARTDLLLPDFLRNLNTDSLSGFLPTLAAGESPAAWPLLSVKVTFFGSGSGVAVSVSVSHKICDIASLVTFVKDWATTTAKGKSNSTIEFAETTIYPPPPSHMYEQFPSTDSDSNITSKYVLKRFVFEPSKIAELKHKAASESVPVPTRVEAIMSLIWRCARNSSRSNLLIPRQAVMWQAMDIRLRIPSSVAPKDVIGNLQSGFSLKKDAESEFEIPEIVATFRKNKERVNEMIKESLQGNTIGQSLLSLMAETVSESTEIDRYIMSSWCRKPFYEVDFGSGSPVWVGYASHTIYDNMVGVVLIDSKEGDGVEAWISLPEEDMSVFVDDQELLAYAVLNPPVVA

SEQ ID NO: 76 - protein, 458 aa, Arabidopsis thaliana
MPMLMATRIDIIQKLNVYPRFQNHDKKKLITLSNLDRQCPLLMYSVFFYKNTTTRDFDSVFSNLKLGLEETMSVWYPAAGRLGLDGGGCKLNIRCNDGGAVMVEAVATGVKLSELGDLTQYNEFYENLVYKPSLDGDFSVMPLVVAQVTRFACGGYSIGIGTSHSLFDGISAYEFIHAWASNSHIHNKSNSKITNKKEDVVIKPVHDRRNLLVNRDAVRETNAAAICHLYQLIKQAMMTYQEQNRNLELPDSGFVIKTFELNGDAIESMKKKSLEGFMCSSFEFLAAHLWKARTRALGLRRDAMVCLQFAVDIRKRTETPLPEGFSGNAYVLASVASTARELLEELTLESIVNKIREAKKSIDQGYINSYMEALGGSNDGNLPPLKELTLISDWTKMPFHNVGFGNGGEPADYMAPLCPPVPQVAYFMKNPKDAKGVLVRIGLDPRDVNGFSNHFLDC